(19) United States
(12) Patent Application Publication
(10) Pub. No.: US 2025/0381213 A1
(43) Pub. Date: Dec. 18, 2025

(54) METHODS FOR LIPID NANOPARTICLE DELIVERY OF CRISPR/CAS SYSTEM

(71) Applicant: Christiana Care Gene Editing Institute, Inc., Wilmington, DE (US)

(72) Inventors: Eric B. Kmiec, Middletown, DE (US); Byung-Chun Yoo, Newark, DE (US); Steven Yang, Chapel Hill, NC (US); Shatovisha Dey, Edgewood, MD (US); Kelly H. Banas, Bear, DE (US)

(73) Assignee: Christiana Care Gene Editing Institute, Inc., Wilmington, DE (US)

(21) Appl. No.: 18/835,913

(22) PCT Filed: Feb. 10, 2023
(86) PCT No.: PCT/US2023/012791

§ 371 Date: Aug. 5, 2024

Related U.S. Application Data
(60) Provisional application No. 63/308,609, filed on Feb. 10, 2022.

(51) Int. Cl. A61K 31/7105; A61K 9/14; A61K 31/282; A61K 31/475; A61K 33/243; A61K 38/46; A61P 35/00; C12N 9/22; C12N 15/113

(52) U.S. Cl. CPC A61K 31/7105; A61K 9/14; A61K 31/282; A61K 31/475; A61K 33/243; A61K 38/465; A61P 35/00; C12N 9/226; C12N 15/1135; C12N 2310/20

(57) ABSTRACT

This disclosure relates to methods treating cancer comprising administering to a subject in need thereof a lipid nanoparticle comprising a therapeutically effective amount of a CRISPR/Cas system comprising one or more nucleic acid sequences encoding one or more guide RNAs (gRNAs) that are complementary to one or more target sequences in a cancer gene of a cancer cell and a nucleic acid sequence encoding a CRISPR-associated endonuclease.

### CROSS-REFERENCE TO RELATED APPLICATIONS

This application is a national stage application filed under 35 U.S.C. § 371 of PCT/US2023/012791, filed Feb. 10, 2023, which claims the benefit of U.S. Provisional Application No. 63/308,609, filed Feb. 10, 2022, each of which is incorporated herein, in its entirety, by reference.

### SUBMISSION OF SEQUENCE LISTING

The Sequence Listing associated with this application is filed in electronic format via Patent Center and hereby incorporated by reference into the specification in its entirety. The name of the text file containing the Sequence Listing is 13094901320SequenceListing.xml. The size of the xml file is 36 KB, and the xml file was created on Feb. 9, 2023.

### FIELD

The field relates to treatment of cancer through lipid nanoparticle delivery of CRISPR/Cas systems.

### BACKGROUND

Gene editing has received attention as a promising method for treatment of numerous gene-associated human diseases such as cancer. With this strategy, a gene is introduced into the targeted tissue or cells by modulating the expression of the genes such as up/down regulate expression and exogenous expression to cure or prevent the progression of the related disease. However, generally, naked genetic molecules, and drug itself show low internalization efficacy in target cells because of their fast degradation in plasma and reduced intracellular uptake by target cells. Further, toxic effect arose by immune response stimulation, leading to the severe limitation of the clinical application. Therefore, carriers that improve the intracellular delivery of nucleic acids is a key factor to establish the delivery technologies.

CRISPR/Cas systems can be delivered to cells in several formats including plasmid DNA encoding the CRISPR-associated protein and guide RNA, and mRNA encoding the CRISPR-associated protein and guide RNA as synthetic molecules that can include chemically modified bases to enhance activity and stability and reduce toxicity. CRISPR-associated proteins and guide RNAs can be delivered as a preassembled ribonucleoprotein (RNP) complex. The benefit of preassembled RNP delivery over plasmid or mRNA are that there is no need to transcribe RNA or translate protein to elicit editing, which can maximize efficiency, and a shorter term of nuclease activity due to RNP decay leading to greater safety and therefore fewer off-target effects.

Lipid-containing nanoparticle compositions, liposomes, and lipoplexes have proven effective as transport vehicles into cells and/or intracellular compartments for biologically active substances such as small molecule drugs, proteins, and nucleic acids. Such compositions generally include one or more cationic and/or amino (ionizable) lipids, phospholipids including polyunsaturated lipids, structural lipids, and/or lipids containing polyethylene glycol. Cationic and/or ionizable lipids include, for example, amine-containing lipids that can be readily protonated. Though a variety of such lipid-containing nanoparticle compositions have been demonstrated, improvements in safety, efficacy, and specificity are still lacking.

The effective targeted delivery of CRISPR/Cas systems represents a continuing medical challenge. In particular, the delivery of CRISPR/Cas systems to cells is made difficult by the relative instability of CRISPR/Cas systems. Thus, there exists a need to develop methods and compositions to facilitate the delivery of CRISPR/Cas systems to cells.

Administering CRISPR/Cas systems to a site of interest with precision has presented an ongoing challenge. Available methods of delivering CRISPR/Cas systems to cells have myriad limitations. For example, adeno-associated viral vectors often used for gene therapy are immunogenic, have a limited payload capacity of <4.6 kb, suffer from poor bio-distribution, can only be administered by direct injection, and pose a risk of disrupting host genes by integration. Non-viral methods have different limitations. Liposomes are primarily delivered to the liver. Extracellular vesicles have a limited payload capacity of <1 kb, limited scalability, and purification difficulties. Thus, there is a recognized need for new methods of delivering CRISPR/Cas systems for treatment of cancer.

### SUMMARY

One aspect is for a method of treating a cancer comprising administering to a subject in need thereof a lipid nanoparticle comprising a therapeutically effective amount of a CRISPR/Cas system comprising (a) one or more nucleic acid sequences encoding one or more guide RNAs (gRNAs) that are complementary to one or more target sequences in a cancer gene of a cancer cell and (b) a nucleic acid sequence encoding a CRISPR-associated endonuclease. In some embodiments, the one or more gRNAs comprise a trans-activated small RNA (tracrRNA) and a CRISPR RNA (crRNA). In some embodiments, the one or more gRNAs are one or more single guide RNAs. In some embodiments, the CRISPR-associated endonuclease is a class 2 CRISPR-associated endonuclease; and in some embodiments, the class 2 CRISPR-associated endonuclease is Cas9 or Cas12a. In some embodiments, the CRISPR/Cas system is comprised in a ribonucleoprotein (RNP) complex. In some embodiments, the cancer is resistant to one or more chemotherapeutic agents. In some embodiments, the cancer is selected from the group consisting of lung cancer, melanoma, esophageal squamous cancer (ESC), head and neck squamous cell carcinoma (HNSCC), and breast cancer; and in some embodiments, the lung cancer is NSCLC. In some embodiments, the method further comprises administering one or more chemotherapeutic agents to the subject; and in some embodiments, the one or more chemotherapeutic agents are selected from the group consisting of cisplatin, vinorelbine, carboplatin, and a combination thereof. In some embodiments, the cancer gene is NRF2 or EGFR.

Another aspect is for a method of reducing expression of a cancer gene in a cancer cell comprising introducing into the cancer cell a lipid nanoparticle comprising (a) one or more nucleic acid sequences encoding one or more guide RNAs (gRNAs) that are complementary to one or more target sequences in the cancer gene and (b) a nucleic acid sequence encoding a CRISPR-associated endonuclease, whereby the one or more gRNAs hybridize to the cancer gene and the CRISPR-associated endonuclease cleaves the cancer gene. In some embodiments, the one or more gRNAs comprise a trans-activated small RNA (tracrRNA) and a CRISPR RNA (crRNA). In some embodiments, the one or more gRNAs are one or more single guide RNAs. In some embodiments, the CRISPR-associated endonuclease is a class 2 CRISPR-associated endonuclease; and in some embodiments, the class 2 CRISPR-associated endonuclease is Cas9 or Cas12a. In some embodiments, activity of the cancer gene is reduced in the cancer cell. In some embodiments, expression or activity of the cancer gene is not completely eliminated in the cancer cell. In some embodiments, expression or activity of the cancer gene is completely eliminated in the cancer cell. In some embodiments, the one or more nucleic acid sequences of (a) and the nucleic acid sequence of (b) is comprised in an RNP complex. In some embodiments, the cancer is resistant to one or more chemotherapeutic agents. In some embodiments, the cancer is selected from the group consisting of lung cancer, melanoma, esophageal squamous cancer (ESC), head and neck squamous cell carcinoma (HNSCC), and breast cancer; and in some embodiments, the lung cancer is NSCLC. In some embodiments, the method further comprises administering one or more chemotherapeutic agents to the subject; and in some embodiments, the one or more chemotherapeutic agents are selected from the group consisting of cisplatin, vinorelbine, carboplatin, and a combination thereof. In some embodiments, the cancer gene is NRF2 or EGFR.

Other objects and advantages will become apparent to those skilled in the art upon reference to the detailed description that hereinafter follows.

### BRIEF DESCRIPTION OF THE DRAWINGS

FIG. 1. Schematic of the workflow for CRISPRMAX or ProdeliverIN-mediated transfections.

FIG. 2. Sequence data from DECODR for (A) H1703 C26-8 and (B) H1703 C44-25 seeded at 0.4×10⁵ for varying ratios of sgRNA to Cas9.

FIG. 3. Sequence data from DECODR for (A) H1703 C26-8 and (B) H1703 C44-25 seeded at varying densities and ratios of sgRNA to Cas9.

FIG. 4. Schematic of the workflow for time-course analysis with CRISPRMAX-mediated genome editing efficiency for sgRNA and Cas9 ratios of 1:1 at 20 pmol concentration.

FIG. 5. Sequence data from DECODR for (A) H1703 C26-8 and (B) H1703 C44-25 for time-course analysis of CRISPRMAX-mediated transfection efficiency for sgRNA and Cas9 ratios of 1:1 at 20 pmol concentration.

FIG. 6. Schematic of the workflow for time-course analysis with CRISPRMAX-mediated genome editing efficiency for sgRNA and Cas9 ratios of 1:1 at 10 pmol concentration.

FIG. 7. Sequence data from DECODR for (A) H1703 C26-8 and (B) H1703 C44-25 for time-course analysis of CRISPRMAX-mediated transfection efficiency for sgRNA and Cas9 ratios of 1:1 at 10 pmol concentration.

FIG. 8. Schematic diagram depicting the experimental setup for assessment of cell viability in response to chemotherapeutic drugs in H1703 WT, H1703 C26-8, and H1703 C44-25 cell lines.

FIG. 9. Chemosensitivity of H1703 WT, H1703 C26-8, and H1703 C44-25 cell lines to (A-C) cisplatin, (D-F) carboplatin, and (G-I) paclitaxel at the indicated concentrations post-CRISPR-Cas9 transfection.

FIG. 10. Intratumoral delivery of luciferase-containing lipid nanoparticles in H1703 44-25 subcutaneous xenograft model. Mice were intratumorally injected with lipid nanoparticles and imaged 4 and 24 hours post injection. The bioluminescent signal from each tumor was quantified by establishing a ROI and each value listed is total flux photons. The scale is representative of all images.

FIG. 11. Intratumoral injection of Fluc LNP in 44-25 cell tumors results in high luciferase expression in tumor samples and minimal biodistribution. qRT-PCR analysis of luciferase expression normalized to Gapdh in (A) Tumor, (B) Lung, (C) Liver, (D) Heart, (E) Ovary, (F) Brain, (G) Spleen, and (H) Blood from NCG mice injected with LNP (n=5 per LNP). Relative luciferase expression, normalized to Gapdh, is graphed as fold change relative to control/un-injected mice. Error bars show +/−standard error.

FIG. 12. Intratumoral delivery of luciferase-containing lipid nanoparticles in a Patient-derived xenograft model. Mice were intratumorally injected with lipid nanoparticles and imaged 4 and 24 hours post injection. The bioluminescent signal from each tumor was quantified by establishing a ROI and each value listed is total flux photons. The scale is representative of all images.

FIG. 13. Intratumoral injection of Fluc LNP in JAX TM00244 tumors results in high luciferase localization in PDX tumor samples and minimal biodistribution. qRT-PCR analysis of luciferase expression normalized to Gapdh in (A) Tumor, (B) Lung, (C) Heart, (D) Kidney, (E) Ovary, (F) Spleen, and (G) Blood from NCG mice injected with LNP (n=3 per LNP). Relative luciferase expression, normalized to Gapdh, is graphed as fold change relative to control/un-injected mice. Error bars show +/−standard error.

FIG. 14. Intratumoral delivery of luciferase-containing lipid nanoparticles in a Patient-derived xenograft model. Mice were intratumorally injected with lipid nanoparticles and imaged 4 and 24 hours post injection. The bioluminescent signal from each tumor was quantified by establishing a ROI and each value listed is total flux photons. The scale is representative of all images.

FIG. 15. Representative images of Cas9 immunostaining of H1703 44-25-derived tumors. The top panel depicts a 20× magnification of a tumor section stained with no primary antibody but all other components, as a control. The bottom panel depicts a 20× magnification of a tumor section stained with Cas9 primary antibody and visualized with DAB stain. The scale bar represents 50 μm.

FIG. 16. Cas9 LNP expression at 72 hours and 1 week post intratumoral injection. qRT-PCR analysis of tumor samples collected 72 hr (A) and 1 week (B) post 10 μg of LNP injection. Relative Cas9 expression, normalized to Gapdh, is graphed as fold change relative to PBS injected mice.

FIG. 17. Genomic analyses of NRF2-targeted tumor tissues from xenograft mouse models. Tumor were homogenized and genomic DNA was extracted. Genomic DNA was analyzed by PCR and Sanger sequencing for the presence of CRISPR activity. Sanger sequencing files were analyzed by the DECODR software, which presents knockout (KO) efficiency, a R² value, INDEL contribution and % of each INDEL contribution. The right hand side displays the raw sequence alignment with respective INDEL contributions as they appear in the Sanger sequence file.

FIG. 18. IL2V62 localization after intratumoral injection is transient. qRT-PCR analysis of luciferase expression normalized to Gapdh at 4, 24 and 1 week in (A) Tumor, (B) Lung, (C) Heart, (D) Kidney, (E) Ovary, (F) Spleen, and (G) Blood from NCG mice injected with LNP (n=3-5 per time point). Tumors were injected with IL2V62 encapsulated with fLuc mRNA. Data were normalized to Gapdh and compared to PBS injected controls. Error bars show +/−standard error.

FIG. 19. Indel analysis of Cas9 activity delivered using LNP. 100 ng, 250 ng, or 500 ng of Cas9 LNP was transfected to H1703 44-25 clonal cells. gDNAs were isolated, Sanger-sequenced, and analyzed using DECODR.

### DETAILED DESCRIPTION

Applicant specifically incorporates the entire contents of all cited references in this disclosure. Further, when an amount, concentration, or other value or parameter is given as either a range or a list of upper values and lower values, this is to be understood as specifically disclosing all ranges formed from any pair of any upper range limit or value and any lower range limit or value, regardless of whether ranges are separately disclosed. Where a range of numerical values is recited herein, unless otherwise stated, the range is intended to include the endpoints thereof, and all integers and fractions within the range. It is not intended that the scope of the present disclosure be limited to the specific values recited when defining a range.

### Definitions

In this disclosure, a number of terms and abbreviations are used. The following definitions are provided.

As used herein, the term “about” or “approximately” means within 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1% or less of a given value or range.

The term “comprising” is intended to include embodiments encompassed by the terms “consisting essentially of” and “consisting of”. Similarly, the term “consisting essentially of” is intended to include embodiments encompassed by the term “consisting of”.

The indefinite articles “a” and “an”, as used herein in the specification and in the claims, unless clearly indicated to the contrary, should be understood to mean “at least one”.

The phrase “and/or”, as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of”, or, when used in the claims, “consisting of”, will refer to the inclusion of exactly one element of a number or list of elements. In general, the term “or” as used herein shall only be interpreted as indicating exclusive alternatives (i.e., “one or the other but not both”) when preceded by terms of exclusivity, “either”, “one of”, “only one of”, “exactly one of”. “Consisting essentially of”, when used in the claims, shall have its ordinary meaning as used in the field of patent law.

An “endonuclease” an enzyme that cleaves the phosphodiester bond within a polynucleotide chain. In some embodiments, an endonuclease generates a double-stranded break at a desired position in the genome, and in some embodiments, an endonuclease generates a single-stranded break or a “nick” or break on one strand of the DNA phosphate sugar backbone at a desired position in the genome, and in some embodiments, without producing undesired off-target DNA stranded breaks. Endonuclease can be naturally occurring endonuclease or it can be artificially generated.

A “Clustered Regularly Interspaced Short Palindromic Repeat (CRISPR)-associated endonuclease protein-binding domain” or “Cas binding domain” refers to a nucleic acid element or domain within a nucleic acid sequence or polynucleotide sequence that, in an effective amount, will bind or have an affinity for one or a plurality of CRISPR-associated endonuclease (or functional fragments thereof). In some embodiments, in the presence of the one or a plurality of proteins (or functional fragments thereof) and a target sequence, the one or plurality of proteins and the nucleic acid element forms a biologically active CRISPR complex and/or can be enzymatically active on a target sequence. In some embodiments, the CRISPR-associated endonuclease is a class 1 or class 2 CRISPR-associated endonuclease, and in some embodiments, a Cas9 or Cas12a endonuclease. The Cas9 endonuclease can have a nucleotide sequence identical to the wild type Streptococcus pyogenes sequence. In some embodiments, the CRISPR-associated endonuclease can be a sequence from other species, for example other Streptococcus species, such as thermophilus; Pseudomonas aeruginosa, Escherichia coli, or other sequenced bacteria genomes and archaea, or other prokaryotic microorganisms. Such species include: Acidovorax avenae, Actinobacillus pleuropneumoniae, Actinobacillus succinogenes, Actinobacillus suis, Actinomyces sp., Alicycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., and Verminephrobacter eiseniae (or functional fragments or variants of any of the aforementioned sequences that have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the aforementioned Cas9 endonucleases). In some embodiments, the CRISPR-associated endonuclease can be a Cas12a nuclease. The Cas12a nuclease can have a nucleotide sequence identical to a wild type Prevotella, Francisella, Acidaminococcus, Proteocatella, Sulfurimonas, Elizabethkingia, Methylococcales, Moraxella, Helcococcus, Lachnospira, Limihaloglobus, Butyrivibrio, Methanomethylophilus, Coprococcus, Synergistes, Eubacterium, Roseburia, Bacteroidales, Ruminococcus, Eubacteriaceae, Leptospira, Parabacteriodes, Gracilibacteria, Lachnospiraceae, Clostridium, Brumimicrobium, Fibrobacter, Catenovulum, Acinetobacter, Flavobacterium, Succiniclasticum, Pseudobutyrivibrio, Barnesiella, Sneathia, Succinivibrionaceae, Treponema, Sedimentisphaera, Thiomicrospira, Eucomonympha, Arcobacter, Oribacterium, Methanoplasma, Porphyromonas, Succinovibrio, or Anaerovibrio sequence (or functional fragments or variants of any of the aforementioned sequences that have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the aforementioned Cas12a endonucleases).

In some embodiments, the terms “(CRISPR)-associated endonuclease protein-binding domain” or “Cas binding domain” refer to a nucleic acid element or domain (e.g. and RNA element or domain) within a nucleic acid sequence that, in an effective amount, will bind to or have an affinity for one or a plurality of CRISPR-associated endonucleases (or functional fragments or variants thereof that are at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to a CRISPR-associated endonuclease). In some embodiments, the Cas binding domain consists of at least or no more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially associates or binds to a biologically active CRISPR-associated endonuclease at a concentration and within a microenvironment suitable for CRISPR system formation.

The “Clustered Regularly Interspaced Short Palindromic Repeats (CRISPR)-CRISPR associated (Cas) (CRISPR-Cas) system guide RNA” or “CRISPR-Cas system guide RNA” may comprise a transcription terminator domain. The term “transcription terminator domain” refers to a nucleic acid element or domain within a nucleic acid sequence (or polynucleotide sequence) that, in an effective amount, prevents bacterial transcription when the CRISPR complex is in a bacterial species and/or creates a secondary structure that stabilizes the association of the nucleic acid sequence to one or a plurality of Cas proteins (or functional fragments thereof) such that, in the presence of the one or a plurality of proteins (or functional fragments thereof), the one or plurality of Cas proteins and the nucleic acid element forms a biologically active CRISPR complex and/or can be enzymatically active on a target sequence in the presence of such a target sequence and a DNA-binding domain. In some embodiments, the transcription terminator domain consists of at least or no more than about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially drives association of the nucleic acid sequence (sgRNA, crRNA with tracrRNA, or other nucleic acid sequence) to a biologically active CRISPR complex at a concentration and microenvironment suitable for CRISPR complex formation.

The term “DNA-binding domain” refers to a nucleic acid element or domain within a nucleic acid sequence (e.g. a guide RNA) that is complementary to a target sequence. In some embodiments, the DNA-binding domain will bind or have an affinity for a target sequence such that, in the presence of a biologically active CRISPR complex, one or plurality of Cas proteins can be enzymatically active on the target sequence. In some embodiments, the DNA binding domain comprises at least one sequence that is capable of forming Watson Crick basepairs with a target sequence as part of a biologically active CRISPR system at a concentration and microenvironment suitable for CRISPR system formation.

“CRISPR system” or “CRISPR/Cas system” refers collectively to transcripts or synthetically produced transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a nucleic acid sequence to which a guide sequence is designed to have complementarity, where hybridization between a target sequence and a guide sequence promotes the formation of a CRISPR complex.

A target sequence may comprise any polynucleotide, such as DNA or RNA polynucleotides. In some embodiments, the target sequence is a DNA polynucleotide and is referred to a DNA target sequence. In some embodiments, a target sequence comprises at least three nucleic acid sequences that are recognized by a Cas-protein when the Cas protein is associated with a CRISPR complex or system which comprises at least one sgRNA or one tracrRNA/crRNA duplex at a concentration and within an microenvironment suitable for association of such a system. In some embodiments, the target DNA comprises at least one or more proto-spacer adjacent motifs which sequences are known in the art and are dependent upon the Cas protein system being used in conjunction with the sgRNA or crRNA/tracrRNAs employed by this work. In some embodiments, the target DNA comprises NNG, where G is an guanine and N is any naturally occurring nucleic acid. In some embodiments the target DNA comprises any one or combination of NNG, NNA, GAA, NGGNG, NGRRT, NGRRN, NNNNGATT, NNNNRYAC, NNAGAAW, TTTV, YG, TTTN, YTN, NGCG, NGAG, NGAN, NGNG, NG, NNGRRT, TYCV, TATV, or NAAAAC. In some embodiments, a target sequence is located in the nucleus or cytoplasm of a cell.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more base pairs from) the target sequence. Without wishing to be bound by theory, the tracr sequence, which may comprise or consist of all or a portion of a wild-type tracr sequence (e.g., about or more than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional (bind the Cas protein or functional fragment thereof). In some embodiments, the tracr sequence has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of sequence complementarity along the length of the tracr mate sequence when optimally aligned. In some embodiments, one or more vectors driving expression of one or more elements of a CRISPR system are introduced into a host cell such that the presence and/or expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. With at least some of the modification contemplated by this disclosure, in some embodiments, the guide sequence or RNA or DNA sequences that form a CRISPR complex are at least partially synthetic. The CRISPR system elements that are combined in a single vector may be arranged in any suitable orientation, such as one element located 5′ with respect to (“upstream” of) or 3′ with respect to (“downstream” of) a second element. In some embodiments, the disclosure relates to a composition comprising a chemically synthesized guide sequence. In some embodiments, the chemically synthesized guide sequence is used in conjunction with a vector comprising a coding sequence that encodes a CRISPR enzyme, such as a class 2 Cas9 or Cas12a protein. In some embodiments, the chemically synthesized guide sequence is used in conjunction with one or more vectors, wherein each vector comprises a coding sequence that encodes a CRISPR enzyme, such as a class 2 Cas9 or Cas12a protein. The coding sequence of one element may be located on the same or opposite strand of the coding sequence of a second element, and oriented in the same or opposite direction. In some embodiments, a single promoter drives expression of a transcript encoding a CRISPR enzyme and one or more additional (second, third, fourth, etc.) guide sequences, tracr mate sequence (optionally operably linked to the guide sequence), and a tracr sequence embedded within one or more intron sequences (e.g., each in a different intron, two or more in at least one intron, or all in a single intron). In some embodiments, the CRISPR enzyme, one or more additional guide sequence, tracr mate sequence, and tracr sequence are each a component of different nucleic acid sequences. For instance, in the case of a tracr and tracr mate sequences and in some embodiments, the disclosure relates to a composition comprising at least a first and second nucleic acid sequence, wherein the first nucleic acid sequence comprises a tracr sequence and the second nucleic acid sequence comprises a tracr mate sequence, wherein the first nucleic acid sequence is at least partially complementary to the second nucleic acid sequence such that the first and second nucleic acid for a duplex and wherein the first nucleic acid and the second nucleic acid either individually or collectively comprise a DNA-targeting domain, a Cas protein binding domain, and a transcription terminator domain. In some embodiments, the CRISPR enzyme, one or more additional guide sequence, tracr mate sequence, and tracr sequence are operably linked to and expressed from the same promoter. In some embodiments, the disclosure relates to compositions comprising any one or combination of the disclosed domains on one guide sequence or two separate tracrRNA/crRNA sequences with or without any of the disclosed modifications. Any methods disclosed herein also relate to the use of tracrRNA/crRNA sequence interchangeably with the use of a guide sequence, such that a composition may comprise a single synthetic guide sequence and/or a synthetic tracrRNA/crRNA with any one or combination of modified domains disclosed herein.

In some embodiments, a guide RNA can be a short, synthetic, chimeric tracrRNA/crRNA (a “single-guide RNA” or “sgRNA”). A guide RNA may also comprise two short, synthetic tracrRNA/crRNAs (a “dual-guide RNA” or “dgRNA”).

As used herein, the term “homologous” or “homologue” or “ortholog” refers to related sequences that share a common ancestor or family member and are determined based on the degree of sequence identity. The terms “homology”, “homologous”, “substantially similar”, and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. In some embodiments, these terms describe the relationship between a gene found in one species, subspecies, variety, cultivar, or strain and the corresponding or equivalent gene in another species, subspecies, variety, cultivar or strain. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania), AlignX (Vector NTI, Invitrogen, Carlsbad, Calif.), and Sequencher (Gene Codes, Ann Arbor, Mich.).

By “hybridizable”, “complementary”, or “substantially complementary” it is meant that a nucleic acid (e.g., RNA, DNA) comprises a sequence of nucleotides that enables it to non-covalently bind, i.e., form Watson-Crick base pairs and/or G/U base pairs, “anneal”, or “hybridize”, to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C). In addition, for hybridization between two RNA molecules (e.g., dsRNA), and for hybridization of a DNA molecule with an RNA molecule (e.g., when a ssRNA target nucleic acid base pairs with a DNA PAMmer, when a DNA target nucleic acid base pairs with an RNA guide nucleic acid, etc.): guanine (G) can also base pair with uracil (U). For example, G/U base-pairing is partially responsible for the degeneracy (i.e., redundancy) of the genetic code in the context of tRNA anti-codon base-pairing with codons in mRNA. Thus, a guanine (G) (e.g., of a protein-binding segment (dsRNA duplex) of a subject guide nucleic acid molecule, of a target nucleic acid base pairing with a guide nucleic acid, and/or a PAMmer, etc.) is considered complementary to both a uracil (U) and to an adenine (A). For example, when a G/U base-pair can be made at a given nucleotide position of a protein-binding segment (e.g., dsRNA duplex) of a subject guide nucleic acid molecule, the position is not considered to be non-complementary, but is instead considered to be complementary.

Hybridization and washing conditions are well known and exemplified in Sambrook J., Fritsch. E. F. and Maniatis, T. Molecular Cloning: A Laboratory Manual, Second Edition. Cold Spring Harbor Laboratory Press. Cold Spring Harbor (1989), particularly Chapter 11 and Table 11.1 therein; and Sambrook. J. and Russell, W., Molecular Cloning: A Laboratory Manual, Third Edition. Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g., complementarity over 35, 34, 33, 32, 31, 30, 29, 28, 27, 26, 25, 24, 23, 22, 21, 20, 19, 18 or less nucleotides), the position of mismatches can become important (see Sambrook et al., supra, 11.7-11.8). Typically, the length for a hybridizable nucleic acid is 8 nucleotides or more (e.g., 10, 11, 12, 13, 14, 15, 16, 17, 18, 19, 20, 21, 22, 23, 24, 25, 26, 27, 28, 29, 30 nucleotides or more). The temperature and wash solution salt concentration may be adjusted as necessary according to factors such as length of the region of complementation and the degree of complementation.

Examples of stringent hybridization conditions include: incubation temperatures of about 25° C., 26° C., 27° C., 28° C., 29° C., 30° C., 31° C., 32° C., 33° C., 34° C., 35° C., 36° C., or 37° C.; hybridization buffer concentrations of about 6×SSC, 7×SSC, 8×SSC, 9×SSC, or 10×SSC; formamide concentrations of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%; and wash solutions from about 4×SSC, 5×SSC, 6×SSC, 7×SSC, to 8×SSC. Examples of moderate hybridization conditions include: incubation temperatures of about 40° C., 41° C., 42° C., 43° C., 44° C., 45° C., 46° C., 47° C., 48° C., 49° C., or 50° C.; buffer concentrations of about 9×SSC, 8×SSC, 7×SSC, 6×SSC, 5×SSC, 4×SSC, 3×SSC, or 2×SSC; formamide concentrations of about 30%, 31%, 32%, 33%, 34%, 35%, 36%, 37%, 38%, 39%, 40%, 41%, 42%, 43%, 44%, 45%, 46%, 47%, 48%, 49%, or 50%; and wash solutions of about 5×SSC, 4×SSC, 3×SSC, or 2×SSC. Examples of high stringency conditions include: incubation temperatures of about 55%, 56%, 57%, 58%, 59%, 60%, 61%, 62%, 63%, 64%, 65%, 66%, 67%, or 68%; buffer concentrations of about 1×SSC, 0.95×SSC, 0.9×SSC, 0.85×SSC, 0.8×SSC, 0.75×SSC, 0.7×SSC, 0.65×SSC, 0.6×SSC, 0.55×SSC, 0.5×SSC, 0.45×SSC, 0.4×SSC, 0.35×SSC, 0.3×SSC, 0.25×SSC, 0.2×SSC, 0.15×SSC, or 0.1×SSC; formamide concentrations of about 55%, 56%, 57%, 58%, 59%, 60%, 61%, 62%, 63%, 64%, 65%, 66%, 67%, 68%, 69%, 70%, 71%, 72%, 73%, 74%, or 75%; and wash solutions of about 1×SSC, 0.95×SSC, 0.9×SSC, 0.85×SSC, 0.8×SSC, 0.75×SSC, 0.7×SSC, 0.65×SSC, 0.6×SSC, 0.55×SSC, 0.5×SSC, 0.45×SSC, 0.4×SSC, 0.35×SSC, 0.3×SSC, 0.25×SSC, 0.2×SSC, 0.15×SSC, or 0.1×SSC, or deionized water. In general, hybridization incubation times are from 5 minutes to 24 hours, with 1, 2, or more washing steps, and wash incubation times are about 1, 2, 3, 4, 5, 6, 7, 8, 9, 10, 11, 12, 13, 14, 15 minutes or more. It is understood that equivalents of SSC using other buffer systems can be employed.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% (i.e., full complementarity) sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90% complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Exemplary methods include BLAST programs (basic local alignment search tools) and PowerBLAST programs (Altschul et al., J. Mol. Biol. 215:403-10 (1990); Zhang et al., Genome Res., 7:649-56 (1997)) or by using the Gap program (Wisconsin Sequence Analysis Package, Version 8 for Unix, Genetics Computer Group, University Research Park, Madison Wis.), using default settings, which uses the algorithm of Smith et al. (Adv. Appl. Math. 2:482-89 (1981)).

As used herein, “cancer” refers to all types of cancer or neoplasm or malignant tumors found in humans, including, but not limited to: leukemias, lymphomas, melanomas, carcinomas and sarcomas. As used herein, the terms or language “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells. In certain embodiments, the cancer is a blood tumor (i.e., a non-solid tumor). In some embodiments, the cancer is lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, or breast invasive carcinoma (see, e.g., Kerins et al., Sci. Rep. 8:12846 (2018)).

In certain embodiments, the cancer is a solid tumor. A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

Specific criteria for the staging of cancer are dependent on the specific cancer type based on tumor size, histological characteristics, tumor markers, and other criteria known by those of skill in the art. Generally, cancer stages can be described as follows:
Stage 0—Carcinoma in situ
Stage I, Stage II, and Stage III—Higher numbers indicate more extensive disease: Larger tumor size and/or spread of the cancer beyond the organ in which it first developed to nearby lymph nodes and/or tissues or organs adjacent to the location of the primary tumor
Stage IV—The cancer has spread to distant tissues or organs

As used herein, a “variant”, “mutant”, or “mutated” polynucleotide contains at least one polynucleotide sequence alteration as compared to the polynucleotide sequence of the corresponding wild-type or parent polynucleotide. Mutations may be natural, deliberate, or accidental. Mutations include substitutions, deletions, and insertions.

As used herein, the terms “treat,” “treating” or “treatment” refer to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) of the state of disease, amelioration or palliation of the disease state, diminishing rate of or time to progression, and remission (whether partial or total). “Treatment” of a cancer can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, a decrease in pain or an increase in the QOL as judged by a qualified individual, e.g., a treating physician, e.g., using accepted assessment tools of pain and QOL is not considered to be a “treatment” of the cancer.

“Chemotherapeutic agent” refers to a drug used for the treatment of cancer. Chemotherapeutic agents include, but are not limited to, small molecules, hormones and hormone analogs, and biologics (e.g., antibodies, peptide drugs, nucleic acid drugs). In certain embodiments, chemotherapy does not include hormones and hormone analogs.

A “cancer that is resistant to one or more chemotherapeutic agents” is a cancer that does not respond, or ceases to respond to treatment with a chemotherapeutic regimen, i.e., does not achieve at least stable disease (i.e., stable disease, partial response, or complete response) in the target lesion either during or after completion of the chemotherapeutic regimen. Resistance to one or more chemotherapeutic agents results in, e.g., tumor growth, increased tumor burden, and/or tumor metastasis.

### CRISPR/Endonucleases

CRISPR/endonuclease (e.g., CRISPR/Cas9) systems are known in the art and are described, for example, in U.S. Pat. No. 9,925,248, which is incorporated by reference herein in its entirety. CRISPR-directed gene editing can identify and execute DNA cleavage at specific sites within the chromosome at a surprisingly high efficiency and precision. The natural activity of CRISPR/Cas9 is to disable a viral genome infecting a bacterial cell. Subsequent genetic reengineering of CRISPR/Cas function in human cells presents the possibility of disabling human genes at a significant frequency.

In bacteria, the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA) containing a DNA binding region (spacer) which is complementary to the target gene.

The compositions described herein can include a nucleic acid encoding a CRISPR-associated endonuclease. The CRISPR-associated endonuclease can be, e.g., a class 1 CRISPR-associated endonuclease or a class 2 CRISPR-associated endonuclease. Class 1 CRISPR-associated endonucleases include type I, type III, and type IV CRISPR-Cas systems, which have effector molecules that comprise multiple subunits. For class 1 CRISPR-associated endonucleases, effector molecules can include, in some embodiments, Cas7 and Cas5, along with, in some embodiments, SS (Cas11) and Cas8a1; Cas8b1; Cas8c; Cas8u2 and Cas6; Cas3″ and Cas10d; Cas SS (Cas11), Cas8e, and Cas6; Cas8f and Cas6f; Cas6f; Cas8-like (Csf1); SS (Cas11) and Cas8-like (Csf1); or SS (Cas11) and Cas10. Class 1 CRISPR-associated endonucleases also be associated with, in some embodiments, target cleavage molecules, which can be Cas3 (type I) or Cas10 (type III) and spacer acquisition molecules such as, e.g., Cas1, Cas2, and/or Cas4. See, e.g., Koonin et al., Curr. Opin. Microbiol. 37:67-78 (2017); Strich et al., J. Clin. Microbiol. 57:1307-18 (2019).

Class 2 CRISPR-associated endonucleases include type I, type V, and type VI CRISPR-Cas systems, which have a single effector molecule. For class 2 CRISPR-associated endonucleases, effector molecules can include, in some embodiments, Cas9, Cas12a (cpf1), Cas12b1 (c2c1), Cas12b2, Cas12c (c2c3), Cas12d (CasY), Cas12e (CasX), Cas12f1 (Cas14a), Cas12f2 (Cas14b), Cas12f3 (Cas14c), Cas12g, Cas12h, Cas12i, Cas12k (c2c5), Cas12j (Cas$), Cas13a (c2c2), Cas13b1 (c2c6), Cas13b2 (c2c6), Cas13c (c2c7), Cas13d, c2c4, c2c8, c2c9, and/or c2c10. See, e.g., Koonin et al., Curr. Opin. Microbiol. 37:67-78 (2017); Strich et al., J. Clin. Microbiol. 57:1307-18 (2019); Makarova et al., Nat. Rev. Microbiol. 18:67-83 (2020); Pausch et al., Science 369:333-37 (2020).

In some embodiments, the CRISPR-associated endonuclease can be a Cas9 nuclease. The Cas9 nuclease can have a nucleotide sequence identical to the wild type Streptococcus pyogenes sequence. In some embodiments, the CRISPR-associated endonuclease can be a sequence from other species, for example other Streptococcus species, such as thermophilus; Pseudomonas aeruginosa, Escherichia coli, or other sequenced bacteria genomes and archaea, or other prokaryotic microorganisms. Such species include: Acidovorax avenae, Actinobacillus pleuropneumoniae, Actinobacillus succinogenes, Actinobacillus suis, Actinomyces sp., Alicycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., and Verminephrobacter eiseniae.

Alternatively, the wild type Streptococcus pyogenes Cas9 sequence can be modified. The nucleic acid sequence can be codon optimized for efficient expression in mammalian cells, e.g., human cells. A Cas9 nuclease sequence codon optimized for expression in human cells sequence can be for example, the Cas9 nuclease sequence encoded by any of the expression vectors listed in Genbank accession numbers KM099231.1 GI: 669193757; KM099232.1 GI: 669193761; or KM099233.1 GI: 669193765. Alternatively, the Cas9 nuclease sequence can be, for example, the sequence contained within a commercially available vector such as pX458, pX330 or pX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI: 669193757; KM099232.1 GI: 669193761; or KM099233.1 GI: 669193765 or Cas9 amino acid sequence of pX458, pX330 or pX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more, e.g., insertions, deletions, or mutations or a combination thereof. One or more of the mutations can be a substitution (e.g., a conservative amino acid substitution). For example, a biologically active variant of a Cas9 polypeptide can have an amino acid sequence with at least or about 50% sequence identity (e.g., at least or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a wild type Cas9 polypeptide.

In some embodiments, the CRISPR-associated endonuclease can be a Cas12a nuclease. The Cas12a nuclease can have a nucleotide sequence identical to a wild type Prevotella or Francisella sequence. Alternatively, a wild type Prevotella or Francisella Cas12a sequence can be modified. In some embodiments, an Acidaminococcus, Proteocatella, Sulfurimonas, Elizabethkingia, Methylococcales, Moraxella, Helcococcus, Lachnospira, Limihaloglobus, Butyrivibrio, Methanomethylophilus, Coprococcus, Synergistes, Eubacterium, Roseburia, Bacteroidales, Ruminococcus, Eubacteriaceae, Leptospira, Parabacteriodes, Gracilibacteria, Lachnospiraceae, Clostridium, Brumimicrobium, Fibrobacter, Catenovulum, Acinetobacter, Flavobacterium, Succiniclasticum, Pseudobutyrivibrio, Barnesiella, Sneathia, Succinivibrionaceae, Treponema, Sedimentisphaera, Thiomicrospira, Eucomonympha, Arcobacter, Oribacterium, Methanoplasma, Porphyromonas, Succinovibrio, or Anaerovibrio Cas12a sequence can be modified. The nucleic acid sequence can be codon optimized for efficient expression in mammalian cells, e.g., human cells. A Cas12a nuclease sequence codon optimized for expression in human cells sequence can be for example, the Cas9 nuclease sequence encoded by any of the expression vectors listed in Genbank accession numbers MF193599.1 GI: 1214941796, KY985374.1 GI: 1242863785, KY985375.1 GI: 1242863787, or KY985376.1 GI: 1242863789. Alternatively, the Cas12a nuclease sequence can be, for example, the sequence contained within a commercially available vector such as pAs-Cpf1 or pLb-Cpf1 from Addgene (Cambridge, Mass.). In some embodiments, the Cas12a endonuclease can have an amino acid sequence that is a variant or a fragment of any of the Cas12a endonuclease sequences of Genbank accession numbers MF193599.1 GI: 1214941796, KY985374.1 GI: 1242863785, KY985375.1 GI: 1242863787, or KY985376.1 GI: 1242863789 or Cas12a amino acid sequence of pAs-Cpf1 or pLb-Cpf1 (Addgene, Cambridge, Mass.). The Cas12a nucleotide sequence can be modified to encode biologically active variants of Cas12a, and these variants can have or can include, for example, an amino acid sequence that differs from a wild type Cas12a by virtue of containing one or more, e.g., insertions, deletions, or mutations or a combination thereof. One or more of the mutations can be a substitution (e.g., a conservative amino acid substitution). For example, a biologically active variant of a Cas12a polypeptide can have an amino acid sequence with at least or about 50% sequence identity (e.g., at least or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a wild type Cas12a polypeptide.

The compositions described herein may also include sequence encoding a guide RNA (gRNA) comprising a DNA-binding domain that is complementary to a target domain in a target sequence, and a CRISPR-associated endonuclease protein-binding domain. The guide RNA sequence can be a sense or anti-sense sequence. The guide RNA sequence may include a PAM. The sequence of the PAM can vary depending upon the specificity requirements of the CRISPR endonuclease used. In, e.g., the CRISPR-Cas system derived from S. pyogenes, the target DNA typically immediately precedes a 5′-NGG proto-spacer adjacent motif (PAM). Thus, for the S. pyogenes Cas9, the PAM sequence can be NGG. Other Cas endonucleases may have different PAM specificities (e.g., NNG, NNA, GAA, NGGNG, NGRRT, NGRRN, NNNNGATT, NNNNRYAC, NNAGAAW, TTTV, YG, TTTN, YTN, NGCG, NGAG, NGAN, NGNG, NG, NNGRRT, TYCV, TATV, or NAAAAC). The specific sequence of the guide RNA may vary, but, regardless of the sequence, useful guide RNA sequences will be those that minimize off-target effects while achieving high efficiency.

In some embodiments, the DNA-binding domain varies in length from about 20 to about 55 nucleotides, for example, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, or about 55 nucleotides. In some embodiments, the Cas protein-binding domain is from about 30 to about 55 nucleotides in length, for example, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, or about 55 nucleotides.

In some embodiments, the compositions comprise one or more nucleic acid (i.e. DNA) sequences encoding the guide RNA and the CRISPR endonuclease. When the compositions are administered as a nucleic acid or are contained within an expression vector, the CRISPR endonuclease can be encoded by the same nucleic acid or vector as the guide RNA sequence. In some embodiments, the CRISPR endonuclease can be encoded in a physically separate nucleic acid from the guide RNA sequence or in a separate vector. The nucleic acid sequence encoding the guide RNA may comprise a DNA binding domain, a Cas protein binding domain, and a transcription terminator domain.

The nucleic acid encoding the guide RNA and/or the CRISPR endonuclease may be an isolated nucleic acid. An “isolated” nucleic acid can be, for example, a naturally-occurring DNA molecule or a fragment thereof, provided that at least one of the nucleic acid sequences normally found immediately flanking that DNA molecule in a naturally-occurring genome is removed or absent. Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids also can be obtained by mutagenesis of, e.g., a naturally occurring portion of a Cas9-encoding DNA (in accordance with, for example, the formula above).

Recombinant constructs are also provided herein and can be used to transform cells in order to express the CRISPR endonuclease and/or a guide RNA complementary to a target sequence. A recombinant nucleic acid construct may comprise a nucleic acid encoding a CRISPR endonuclease and/or a guide RNA complementary to a target sequence, operably linked to a promoter suitable for expressing the CRISPR endonuclease and/or a guide RNA complementary to the target sequence in the cell. In some embodiments, the nucleic acid encoding a CRISPR endonuclease is operably linked to the same promoter as the nucleic acid encoding the guide RNA. In other embodiments, the nucleic acid encoding a CRISPR endonuclease and the nucleic acid encoding the guide RNA are operably linked to different promoters. In some embodiments, the promoter can be one or more pol III promoters, one or more pol II promoters, one or more pol I promoters, or combinations thereof. Examples of pol III promoters include, but are not limited to, U6 and H1 promoters. Examples of pol II promoters include, but are not limited to, the retroviral Rous sarcoma virus (RSV), LTR promoter (optionally with the RSV enhancer), the cytomegalovirus (CMV) promoter (optionally with the CMV enhancer; see, e.g., Boshart et al., Cell 41:521-30 (1985)), the SV40 promoter, the dihydrofolate reductase promoter, the β-actin promoter, the phosphoglycerol kinase (PGK) promoter, and the EFla promoter. An example of a pol I promoter includes, but is not limited to, the 47S pre-rRNA promoter.

### Lipid Nanoparticle Delivery

Introduction of CRISPR/Cas systems can be accomplished by lipid nanoparticle (LNP)-mediated delivery. For example, LNP-mediated delivery can be used to deliver a combination of Cas mRNA and guide RNA or a combination of Cas protein and guide RNA. Delivery through such methods results in transient Cas expression, and the biodegradable lipids improve clearance, improve tolerability, and decrease immunogenicity. Lipid formulations can protect biological molecules from degradation while improving their cellular uptake.

LNPs are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1, herein incorporated by reference in its entirety for all purposes. An exemplary lipid nanoparticle can comprise a cationic lipid and one or more other components. In one embodiment, the other component can comprise a helper lipid such as cholesterol. In another embodiment, the other components can comprise a helper lipid such as cholesterol and a neutral lipid such as distearoylphosphatidylcholine (DSPC).

An LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al., Cell Rep. 22:1-9 (2018) and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an exogenous donor nucleic acid. In certain LNPs, the cargo can include a guide RNA or a nucleic acid encoding a guide RNA and a Cas protein or a nucleic acid encoding a Cas protein. In certain LNPs, the cargo can include a guide RNA or a nucleic acid encoding a guide RNA, a Cas protein or a nucleic acid encoding a Cas protein, and an exogenous donor nucleic acid.

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy) butanoyl)oxy)-2-((((3-(diethylamino) propoxy-) carbonyl)oxy)methyl) propyl octadeca-9,12-dienoate, also called 3-((4,4-bis(octyloxy) butanoyl)oxy)-2-((((3-(diethylamino) propoxy) carbonyl-)oxy)methyl) propyl (9Z,12Z)-octadeca-9,12-dienoate. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino) propoxy) carbonyl)oxy) hexadecanoyl)oxy) propane-1-,3-diyl(9Z,9Z′,12Z,12Z′)-bis(octadeca-9,12-dienoate). Another example of a suitable lipid is Lipid D, which is 3-(((3-(dimethylamino) propoxy) carbonyl)oxy)-13-(octanoyloxy)tridecyl 3-octylundecanoate. Other suitable lipids include heptatriaconta-6,9,28,31-tetraen-19-yl 4-(dimethylamino) butanoate (also known as Dlin-MC3-DMA (MC3))).

Cationic lipid can be present in embodiments of the composition and lipid particles can comprise an amount from about 30 to about 60 mole percent (“mol %”, or the percentage of the total moles that is of a particular component), from about 30 mol % to about 55 mol %, from about 30 mol % to about 50 mol %, from about 30 mol % to about 45 mol %, from about 30 mol % to about 40 mol %, from about 30 mol % to about 35 mol %, from about 35 mol % to about 60 mol %, from about 40 mol % to about 60 mol %, from about 45 mol % to about 60 mol %, from about 50 mol % to about 60 mol %, from about 55 mol % to about 60 mol %, from about 35 mol % to about 55 mol %, from about 40 mol % to about 50 mol %. In in some embodiments, the cationic lipid is present in about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

Some such lipids suitable for use in the LNPs described herein are biodegradable in vivo. For example, LNPs comprising such a lipid include those where at least 75% of the lipid is cleared from the plasma within 8, 10, 12, 24, or 48 hours, or 3, 4, 5, 6, 7, or 10 days. As another example, at least 50% of the LNP is cleared from the plasma within 8, 10, 12, 24, or 48 hours, or 3, 4, 5, 6, 7, or 10 days.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral (also termed structural) lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and combinations thereof. For example, the neutral phospholipid may be selected from the group consisting of distearoylphosphatidylcholine (DSPC) and dimyristoyl phosphatidyl ethanolamine (DMPE).

In certain embodiments, a neutral lipid is present in the lipid particle in an amount from about 20 mol % to about 40 mol %, from about 20 mol % to about 35 mol %, from about 20 mol % to about 30 mol %, from about 20 mol % to about 25 mol %, from about 25 mol % to about 40 mol %, from about 30 mol % to about 40 mol %, from about 30 mol % to about 40 mol %, from about 35 mol % to about 40 mol %, from about 25 mol % to about 35 mol %. In in some embodiments, the cationic lipid is present in about 20, 21, 22, 23, 24, 25, 26, 27, 28, 29, 30, 31, 32, 33, 34, 35, 36, 37, 38, 39, or 40 mol %.

In some embodiments, the lipids can be any of the lipids disclosed in US20210251898, US20210220449, US20210128488, US20210122703, US20210122702, US20210113483, US20210107861, US20210095309, US20210087135, US20190292566 each incorporated herein by reference in its entirety.

Commercially available LNPs include, e.g., Lipofectamine™ CRISPRMAX™ Cas9 Transfection Reagent (available from ThermoFisher Scientific, Waltham, MA), Pro-DeliverIN™ CRISPR Transfection Reagent (available from Oz Biosciences, San Diego, CA), and NanoAssemblr® LNPs (available from Precision NanoSystems, Vancouver, BC).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety. The hydrophilic head group of stealth lipid can comprise, for example, a polymer moiety selected from polymers based on poly(ethylene glycol), poly(oxazoline), poly(vinyl alcohol), poly(glycerol), poly(N-vinylpyrrolidone), polyaminoacids, and poly N-(2-hydroxypropyl) methacrylamide.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

In some embodiments, the stealth lipid may be PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3 [beta]-oxy) carboxamido-3′,6′-dioxaoctanyl]carbamoyl-ω-methyl-poly(ethylene glycol), PEG-DMB (3,4-ditetradecoxylbenzyl-ω-methyl-poly(ethylene glycol) ether), 1,2-dimyristoyl-sn-glycero-3-phosphoethanolamine-N-[methoxy (polyethylene glycol)-2000] (PEG2k-DMG), 1,2-distearoyl-sn-glycero-3-phosphoethanolamine-N-[methoxy (polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), or 1,2-distearyloxypropyl-3-amine-N-[methoxy (polyethylene glycol)-2000] (PEG2k-DSA).

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5.

In some LNPs, the cargo can comprise Cas mRNA and gRNA. The Cas mRNA and gRNAs can be in different ratios. For example, the LNP formulation can include a ratio of Cas mRNA to gRNA nucleic acid ranging from about 25:1 to about 1:25, ranging from about 10:1 to about 1:10, ranging from about 5:1 to about 1:5, or about 1:1. Alternatively, the LNP formulation can include a ratio of Cas mRNA to gRNA nucleic acid from about 1:1 to about 1:5, or about 10:1. Alternatively, the LNP formulation can include a ratio of Cas mRNA to gRNA nucleic acid of about 1:10, 25:1, 10:1, 5:1, 3:1, 1:1, 1:3, 1:5, 1:10, or 1:25.

In some LNPs, the cargo can comprise exogenous donor nucleic acid and gRNA. The exogenous donor nucleic acid and gRNAs can be in different ratios. For example, the LNP formulation can include a ratio of exogenous donor nucleic acid to gRNA nucleic acid ranging from about 25:1 to about 1:25, ranging from about 10:1 to about 1:10, ranging from about 5:1 to about 1:5, or about 1:1. Alternatively, the LNP formulation can include a ratio of exogenous donor nucleic acid to gRNA nucleic acid from about 1:1 to about 1:5, about 5:1 to about 1:1, about 10:1, or about 1:10. Alternatively, the LNP formulation can include a ratio of exogenous donor nucleic acid to gRNA nucleic acid of about 1:10, 25:1, 10:1, 5:1, 3:1, 2.5:1, 1:1, 1:2.5, 1:3, 1:5, 1:10, or 1:25.

In some embodiments, one or more CRISPR endonucleases and one or more guide RNAs may be provided in combination in the form of ribonucleoprotein particles (RNPs). An RNP complex can be introduced into a subject by means of, e.g., injection, electroporation, nanoparticles (including, e.g., lipid nanoparticles), vesicles, and/or with the assistance of cell-penetrating peptides. See, e.g., Lin et al., ELife 3: e04766 (2014); Sansbury et al., CRISPR J. 2 (2): 121-32 (2019); US2019/0359973)

LNP particles can have a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm. LNPs may be made from cationic, anionic, or neutral lipids. Neutral lipids, such as the fusogenic phospholipid DOPE or the membrane component cholesterol, may be included in LNPs as “helper lipids” to enhance transfection activity and nanoparticle stability. LNPs may also be comprised of hydrophobic lipids, hydrophilic lipids, or both hydrophobic and hydrophilic lipids.

In certain embodiments, the cationic lipid N-[1-(2,3-dioleyloxy) propyl]-N,N,N-trimethylammonium chloride (DOTMA) can be used. DOTMA can be formulated alone or combined with the neutral lipid, dioleoylphosphatidyl-ethanolamine (DOPE) or other cationic or non-cationic lipids into a liposomal transfer vehicle or a lipid nanoparticle, and such liposomes can be used to enhance the delivery of nucleic acids into target cells. Other suitable cationic lipids include, but are not limited to, 5-carboxyspermylglycinedioctadecylamide, 2,3-dioleyloxy-N-[2 (spermine-carboxamido)ethyl]-N,N-dimethyl-1-propanaminium, 1,2-Dioleoyl-3-Dimethylammonium-Propane, 1,2-Dioleoyl-3-Trimethylammonium-Propane. Contemplated cationic lipids also include 1,2-distearyloxy-N,N-dimethyl-3-aminopropane, 1,2-dioleyloxy-N,N-dimethyl-3-aminopropane, 1,2-dilinoleyloxy-N,N-dimethyl-3-aminopropane, 1,2-dilinolenyloxy-N, N-dimethyl-3-aminopropane, N-dioleyl-N,N-dimethylammonium chloride, N,N-distearyl-N,N-dimethylammonium bromide, N-(1,2-dimyristyloxyprop-3-yl)-N,N-dimethyl-N-hydroxyethyl ammonium bromide, 3-dimethylamino-2-(cholest-5-en-3-beta-oxybutan-4-oxy)-1-(cis, ci-s-9,12-octadecadienoxy) propane, 2-[5′-(cholest-5-en-3-beta-oxy)-3′-oxapentoxy)-3-dimethyl-1-(cis,cis-9′,12′-octadecadienoxy) propane, N,N-dimethyl-3,4-dioleyloxybenzylamine, 1,2-N,N′-dioleylcarbamyl-3-dimethylaminopropane, 2,3-Dilinoleoyloxy-N,N-dimethylpropylamine, 1,2-N,N′-Dilinoleylcarbamyl-3-dimethylaminopropane, 1,2-Dilinoleoylcarbamyl-3-dimethylaminopropane, 2,2-dilinoleyl-4-dimethylaminomethyl-[1,3]-dioxolane, 2,2-dilinoleyl-4-dimethylaminoethyl-[1,3]-dioxolane, and 2-(2,2-di((9Z,12Z)-octadeca-9,12-dien-1-yl)-1,3-dioxolan-4-yl)-N,N-dimethylethanamine (DLin-KC2-DMA)), or mixtures thereof.

In some embodiments, non-cationic lipids can be used. As used herein, the phrase “non-cationic lipid” refers to any neutral, zwitterionic, or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), DOPE, palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone or can be used in combination with other excipients, for example, cationic lipids.

DNA vectors containing nucleic acids such as those described herein also are also provided. A “DNA vector” is a replicon, such as a plasmid, phage, or cosmid, into which another DNA segment may be inserted so as to bring about the replication of the inserted segment. Generally, a DNA vector is capable of replication when associated with the proper control elements. Suitable vector backbones include, for example, those routinely used in the art such as plasmids, viruses, artificial chromosomes, BACs, YACs, or PACs. The term “DNA vector” includes cloning and expression vectors, as well as viral vectors and integrating vectors. An “expression vector” is a vector that includes a regulatory region. A wide variety of host/expression vector combinations may be used to express the nucleic acid sequences described herein. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The DNA vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). As noted above, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

The DNA vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region (e.g. a promoter) and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Direct injection of adenoviral vectors into lung tumors has been a routine procedure in clinical trials evaluating gene therapy of lung cancer. Dong et al., J. Int. Med. Res. 36:1273-87 (2008); Li et al., Cancer Gene Ther. 20:251-59 (2013); Zhou et al., Cancer Gene Ther. 23:1-6 (2016). Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al., BioTechniques 34:167-71 (2003). A large variety of such vectors are known in the art and are generally available.

Suitable nucleic acid delivery systems include recombinant viral vector, typically sequence from at least one of an adenovirus, adenovirus-associated virus (AAV), helper-dependent adenovirus, retrovirus, or hemagglutinating virus of Japan-liposome (HVJ) complex. In such cases, the viral vector comprises a strong eukaryotic promoter operably linked to the polynucleotide e.g., a cytomegalovirus (CMV) promoter. The recombinant viral vector can include one or more of the polynucleotides therein, in some embodiments about one polynucleotide. LNPs may be administered to a subject subcutaneously, intradermally, intratumorally, intranodally, intramedullary, intramuscularly, intrathecally, by intravenous or intralymphatic injection, or intraperitoneally.

In embodiments in which the polynucleotide is to be administered with a non-viral vector, use of between from about 0.1 ng to about 4000 μg will often be useful e.g., about 0.1 ng to about 3900 μg, about 0.1 ng to about 3800 μg, about 0.1 ng to about 3700 μg, about 0.1 ng to about 3600 μg, about 0.1 ng to about 3500 μg, about 0.1 ng to about 3400 μg, about 0.1 ng to about 3300 μg, about 0.1 ng to about 3200 μg, about 0.1 ng to about 3100 μg, about 0.1 ng to about 3000 μg, about 0.1 ng to about 2900 μg, about 0.1 ng to about 2800 μg, about 0.1 ng to about 2700 μg, about 0.1 ng to about 2600 μg, about 0.1 ng to about 2500 μg, about 0.1 ng to about 2400 μg, about 0.1 ng to about 2300 μg, about 0.1 ng to about 2200 μg, about 0.1 ng to about 2100 μg, about 0.1 ng to about 2000 μg, about 0.1 ng to about 1900 μg, about 0.1 ng to about 1800 μg, about 0.1 ng to about 1700 μg, about 0.1 ng to about 1600 μg, about 0.1 ng to about 1500 μg, about 0.1 ng to about 1400 μg, about 0.1 ng to about 1300 μg, about 0.1 ng to about 1200 μg, about 0.1 ng to about 1100 μg, about 0.1 ng to about 1000 μg, about 0.1 ng to about 900 μg, about 0.1 ng to about 800 μg, about 0.1 ng to about 700 μg, about 0.1 ng to about 600 μg, about 0.1 ng to about 500 μg, about 0.1 ng to about 400 μg, about 0.1 ng to about 300 μg, about 0.1 ng to about 200 μg, about 0.1 ng to about 100 μg, about 0.1 ng to about 90 μg, about 0.1 ng to about 80 μg, about 0.1 ng to about 70 μg, about 0.1 ng to about 60 μg, about 0.1 ng to about 50 μg, about 0.1 ng to about 40 μg, about 0.1 ng to about 30 μg, about 0.1 ng to about 20 μg, about 0.1 ng to about 10 μg, about 0.1 ng to about 1 μg, about 0.1 ng to about 900 ng, about 0.1 ng to about 800 ng, about 0.1 ng to about 700 ng, about 0.1 ng to about 600 ng, about 0.1 ng to about 500 ng, about 0.1 ng to about 400 ng, about 0.1 ng to about 300 ng, about 0.1 ng to about 200 ng, about 0.1 ng to about 100 ng, about 0.1 ng to about 90 ng, about 0.1 ng to about 80 ng, about 0.1 ng to about 70 ng, about 0.1 ng to about 60 ng, about 0.1 ng to about 50 ng, about 0.1 ng to about 40 ng, about 0.1 ng to about 30 ng, about 0.1 ng to about 20 ng, about 0.1 ng to about 10 ng, about 0.1 ng to about 1 ng, about 1 ng to about 4000 μg, about 1 ng to about 3900 μg, about 1 ng to about 3800 μg, about 1 ng to about 3700 μg, about 1 ng to about 3600 μg, about 1 ng to about 3500 μg, about 1 ng to about 3400 μg, about 1 ng to about 3300 μg, about 1 ng to about 3200 μg, about 1 ng to about 3100 μg, about 1 ng to about 3000 μg, about 1 ng to about 2900 μg, about 1 ng to about 2800 μg, about 1 ng to about 2700 μg, about 1 ng to about 2600 μg, about 1 ng to about 2500 μg, about 1 ng to about 2400 μg, about 1 ng to about 2300 μg, about 1 ng to about 2200 μg, about 1 ng to about 2100 μg, about 1 ng to about 2000 μg, about 1 ng to about 1900 μg, about 1 ng to about 1800 μg, about 1 ng to about 1700 μg, about 1 ng to about 1600 μg, about 1 ng to about 1500 μg, about 1 ng to about 1400 μg, about 1 ng to about 1300 μg, about 1 ng to about 1200 μg, about 1 ng to about 1100 μg, about 1 ng to about 1000 μg, about 1 ng to about 900 μg, about 1 ng to about 800 μg, about 1 ng to about 700 μg, about 1 ng to about 600 μg, about 1 ng to about 500 μg, about 1 ng to about 400 μg, about 1 ng to about 300 μg, about 1 ng to about 200 μg, about 1 ng to about 100 μg, about 1 ng to about 90 μg, about 1 ng to about 80 μg, about 1 ng to about 70 μg, about 1 ng to about 60 μg, about 1 ng to about 50 μg, about 1 ng to about 40 μg, about 1 ng to about 30 μg, about 1 ng to about 20 μg, about 1 ng to about 10 μg, about 1 ng to about 1 μg, about 1 ng to about 900 ng, about 1 ng to about 800 ng, about 1 ng to about 700 ng, about 1 ng to about 600 ng, about 1 ng to about 500 ng, about 1 ng to about 400 ng, about 1 ng to about 300 ng, about 1 ng to about 200 ng, about 1 ng to about 100 ng, about 1 ng to about 90 ng, about 1 ng to about 80 ng, about 1 ng to about 70 ng, about 1 ng to about 60 ng, about 1 ng to about 50 ng, about 1 ng to about 40 ng, about 1 ng to about 30 ng, about 1 ng to about 20 ng, about 1 ng to about 10 ng, about 10 ng to about 4000 μg, about 20 ng to about 4000 μg, about 30 ng to about 4000 μg, about 40 ng to about 4000 μg, about 50 ng to about 4000 μg, about 60 ng to about 4000 μg, about 70 ng to about 4000 μg, about 80 ng to about 4000 μg, about 90 ng to about 4000 μg, about 100 ng to about 4000 μg, about 200 ng to about 4000 μg, about 300 ng to about 4000 μg, about 400 ng to about 4000 μg, about 500 ng to about 4000 μg, about 600 ng to about 4000 μg, about 700 ng to about 4000 μg, about 800 ng to about 4000 μg, about 900 ng to about 4000 μg, about 1 μg to about 4000 μg, 10 μg to about 4000 μg, 20 μg to about 4000 μg, 30 μg to about 4000 μg, 40 μg to about 4000 μg, 50 μg to about 4000 μg, 60 μg to about 4000 μg, 70 μg to about 4000 μg, 80 μg to about 4000 μg, 90 μg to about 4000 μg, 100 μg to about 4000 μg, 200 μg to about 4000 μg, 300 μg to about 4000 μg, 400 μg to about 4000 μg, 500 μg to about 4000 μg, 600 μg to about 4000 μg, 700 μg to about 4000 μg, 800 μg to about 4000 μg, 900 μg to about 4000 μg, 1000 μg to about 4000 μg, 1100 μg to about 4000 μg, 1200 μg to about 4000 μg, 1300 μg to about 4000 μg, 1400 μg to about 4000 μg, 1500 μg to about 4000 μg, 1600 μg to about 4000 μg, 1700 μg to about 4000 μg, 1800 μg to about 4000 μg, 1900 μg to about 4000 μg, 2000 μg to about 4000 μg, 2100 μg to about 4000 μg, 2200 μg to about 4000 μg, 2300 μg to about 4000 μg, 2400 μg to about 4000 μg, 2500 μg to about 4000 μg, 2600 μg to about 4000 μg, 2700 μg to about 4000 μg, 2800 μg to about 4000 μg, 2900 μg to about 4000 μg, 3000 μg to about 4000 μg, 3100 μg to about 4000 μg, 3200 μg to about 4000 μg, 3300 μg to about 4000 μg, 3400 μg to about 4000 μg, 3500 μg to about 4000 μg, 3600 μg to about 4000 μg, 3700 μg to about 4000 μg, 3800 μg to about 4000 μg, or 3900 μg to about 4000 μg.

Additional vectors include viral vectors, fusion proteins and chemical conjugates. Retroviral vectors include Moloney murine leukemia viruses and HIV-based viruses. One HIV-based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector (see, e.g., Geller et al., J. Neurochem. 64:487-96 (1995); Lim et al., in DNA Cloning: Mammalian Systems, D. Glover, Ed. (Oxford Univ. Press, Oxford England) (1995); Geller et al., Proc. Natl. Acad. Sci. USA 90:7603-07 (1993); Geller et al., Proc. Natl. Acad. Sci. USA 87:1149-53 (1990)), Adenovirus Vectors (see, e.g., Le Gal LaSalle et al., Science 259:988-90 (1993); Davidson et al., Nat. Genet. 3:219-23 (1993); Yang et al., J. Virol. 69:2004-15 (1995)), and Adeno-associated Virus Vectors (see, e.g., Kaplitt et al., Nat. Genet. 8:148-54 (1994)).

If desired, the polynucleotides described here may also be used with a microdelivery vehicle such as cationic liposomes, adenoviral vectors, and exosomes. For a review of the procedures for liposome preparation, targeting and delivery of contents, see Mannino et al., BioTechniques 6:682-90 (1988). See also Feigner et al., Bethesda Res. Lab. Focus 11 (2): 21 (1989) and Maurer, Bethesda Res. Lab. Focus 11 (2): 25 (1989). In some embodiments, exosomes may be used for delivery of a nucleic acid encoding a CRISPR endonuclease and/or guide RNA to a target cell, e.g. a cancer cell. Exosomes are nanosized vesicles secreted by a variety of cells and are comprised of cellular membranes. Exosomes can attach to target cells by a range of surface adhesion proteins and vector ligands (tetraspanins, integrins, CD11b and CD18 receptors), and deliver their payload to target cells. Several studies indicate that exosomes have a specific cell tropism, according to their characteristics and origin, which can be used to target them to disease tissues and/or organs. See Batrakova et al., J. Control. Release 219:396-405 (2015). For example, cancer-derived exosomes function as natural carriers that can efficiently deliver CRISPR/Cas9 plasmids to cancer cells. See Kim et al., J. Control. Release 266:8-16 (2017).

Replication-defective recombinant adenoviral vectors, can be produced in accordance with known techniques. See Quantin et al., Proc. Natl. Acad. Sci. USA 89:2581-84 (1992); Stratford-Perricadet et al., J. Clin. Invest. 90:626-30 (1992); Rosenfeld et al., Cell 68:143-55 (1992).

Another delivery method is to use single stranded DNA producing vectors which can produce the expressed products intracellularly. See, e.g., Chen et al., BioTechniques, 34:167-71 (2003).

### Cancer Genes

In some embodiments, the cancer gene is NRF2, EGFR, EIF1AX, GNA11, SF3B1, BAP1, PBRM1, ATM, SETD2, KDM6A, CUL3, MET, SMARCA4, U2AF1, RBM10, STK11, NF1, NF2, IDH1, IDH2, PTPN11, MAX, TCF12, HIST1H1E, LZTR1, KIT, RAC1, ARID2, BRD4, BRD7, BARF1, NRAS, RNF43, SMAD4, ARID1A, ARID1B, KRAS, APC, SMAD2, SMAD3, ACVR2A, GNAS, HRAS, STAG2, FGFR3, FGFR4, RHOA, CDKN1A, ERBB3, KANSL1, RB1, TP53, CDKN2A, CDKN2B, CDKN2C, KEAP1, CASP8, TGFBR2, HLA-B, MAPK1, NOTCH1, NOTCH2, NOTCH3, HLA-A, RASA1, EPHA2, EPHA3, EPHA5, EPHA7, NSD1, ZNF217, ZNF750, KLF5, EP300, FAT1, PTEN, FBXW7, PIK3CA, PIK3CB, PIK3C2B, PIK3CG, RUNX1, RUNX1T1, DNMT3A, SMC1A, ERBB2, AKT1, AKT2, AKT3, MAP3K1, FOXA1, BRCA1, BRCA2, CDH1, PIK3R1, PPP2R1A, BCOR, BCORL1, ARHGAP35, FGFR2, CHD4, CTCF, CTNNA1, CTNNB1, SPOP, TMSB4X, PIM1, CD70, CD79A, CD79B, B2M, CARD11, MYD88, BTG1, BTG2, TNFAIP3, MEN1, PRKAR1A, PDGFRA, PDGFRB, SPTA1, GABRA6, KEL, SMARCB1, ZBTB7B, BCL2, BCL2L1, BCL2L2, BCL2L11, RFC1, MAP3K4, CSDE1, EPAS1, RET, LATS2, EEF2, CYLD, HUWE1, MYH9, AJUBA, FLNA, ERBB4, CNBD1, DMD, MUC6, FAM46C, FAM46D, PLCG1, PLCG2, NIPBL, FUBP1, CIC, ZBTB2, ZBTB20, ZCCHC12, TGIF1, SOX2, SOX9, SOX10, PCBP1, ZFP36L2, TCF7L2, AMER1, KDM5A, KDM5C, MTOR, VHL, KIF1A, TCEB1, TXNIP, CUL1, TSC1, ELF3, RHOB, PSIP1, SF1, FOXQ1, GNA13, DIAPH2, ZFP36L1, ERCC2, SPTAN1, RXRA, ASXL2, CREBBP, CREB3L3, ALB, DHX9, XPO1, RPS6KA3, IL6ST, TSC2, EEF1A1, WHSC1, APOB, NUP133, AXIN1, PHF6, TET2, WT1, FLT3, FLT4, SMC3, CEBPA, RAD21, RAD50, RAD51, PTPDC1, ASXL1, EZH2, NPM1, SRSF2, GNAQ, PLCB4, CYSLTR2, CDKN1B, CBFB, NCOR1, PTPRD, TBX3, GPS2, GATA1, GATA2, GATA3, GATA4, GATA6, MAP2K4, PTCH1, PTMA, LATS1, POLRMT, CDK4, COL5A1, PPP6C, MECOM, DACH1, MAP2K1, MAP2K2, RQCD1, DDX3X, NUP93, PPM1D, CHD2, CHD3, CCND1, CCND2, CCND3, ACVR1, KMT2A, KMT2B, KMT2C, KMT2D, SIN3A, SCAF4, DICER1, FOXA2, CTNND1, MYC, MYCL, MYCN, SOX17, ARID5B, ATR, INPPL1, INPP4B, ATF7IP, ZMYM2, ZFHX3, PDS5B, SOS1, TAF1, PIK3R2, RPL22, RRAS2, MSH2, MSH6, CKD12, ZNF133, ZNF703, MED12, ZMYM3, GTF21, RIT1, MGA, ABL1, BRAF, CHEK1, FANCC, JAK2, MITF, PDCD1LG2, STAT4, ABL2, CHEK2, FANCD2, JAK3, MLH1, FANCE, JUN, MPL, RICTOR, SUFU, FANCF, GID4, KAT6A, MRE11A, PDK1, SYK, BRIP1, CRKL, FANCG, GLI1, CRLF2, FANCL, RPTOR, ALK, BTK, CSF1R, FAS, TERC, C11orf30, KDR, MUTYH, SDHA, AR, FGF10, GPR124, SDHB, ARAF, CBL, FGF14, GRIN2A, SDHC, ARFRP1, FGF19, GRM3, KLHL6, PMS2, SDHD, TNFRSF14, DAXX, FGF23, GSK3B, POLD1, TOP1, DDR2, FGF3, H3F3A, POLE, TOP2A, CCNE1, FGF4, HGF, SLIT2, CD274, FGF6, HNF1A, NFKBIA, PRDM1, DOT1L, FGFR1, LMO1, NKX2-1, PREX2, HSD3B1, LRP1B, TSHR, ATRX, CDC73, HSP90AA1, PRKCI, AURKA, PRKDC, VEGFA, AURKB, CDK12, FH, MAGI2, PRSS8, SMO, FLCN, IGF1R, SNCAIP, WISP3, AXL, CDK6, EPHB1, FLT1, IGF2, SOCS1, CDK8, IKBKE, NTRK1, BARD1, IKZF1, NTRK2, QKI, FOXL2, IL7R, MCL1, NTRK3, ERG, FOXP1, INHBA, MDM2, SPEN, ERRFI1, FRS2, MDM4, PAK3, BCL6, ESR1, IRF2, PALB2, RAF1, IRF4, MEF2B, PARK2, RANBP2, SRC, IRS2, PAX5, RARA, BLM, FANCA, JAK1, FCRL4, LIG4, MAR, PWWP3A, MUC16, MUC17, FCGBP, FAT17, MMSET, IRTA2, TTN, DST, or STAT3.

In some embodiments, the cancer gene is Nuclear Factor Erythroid 2-Related Factor (NRF2, NFE2L2). NRF2 is considered the master regulator of 100-200 target genes involved in cellular responses to oxidative/electrophilic stress. Targets include glutathione (GSH) mediators, antioxidants and genes controlling efflux pumps. (Hayden et al., Urol. Oncol. Semin. Orig. Investig. 32:806-14 (2014)). NRF2 is also known to regulate expression of genes involved in protein degradation and detoxification and is negatively regulated by Kelch-like ECH-associated protein 1 (KEAP1), a substrate adapter for the Cul3-dependent E3 ubiquitin ligase complex. Under normal conditions, Keap1 constantly targets NRF2 for ubiquitin-dependent degradation maintaining low expression of NRF2 on downstream target genes. However, chemotherapy has been shown to activate transcriptional activity of the NRF2 target genes often triggering a cytoprotective response; enhanced expression of NRF2 occurs in response to environmental stress or detrimental growth conditions. Other mechanisms that lead to NRF2 upregulation include mutations in KEAP1 or epigenetic changes of the promoter region. The upregulation of NRF2 expression leads to an enhanced resistance of cancer cells to chemotherapeutic drugs, which by their very action induce an unfavorable environment for cell proliferation. Indeed, Hayden et al. (ibid) have clearly demonstrated that increased NRF2 expression leads to the resistance of cancer cells to chemotherapeutic drugs including cisplatin. Singh et al. (2010, Antioxidants & Redox Signaling 13) also showed that constitutive expression of NRF2 leads to radioresistance, and inhibition of NRF2 causes increased endogenous reactive oxygen species (ROS) levels as well as decreased survival. Torrente et al. (Oncogene (2017). doi: 10.1038/onc.2017.221) identified crosstalk between NRF2 and the homeodomain interacting protein kinase two, HIPK2, demonstrating that HIPK2 exhibits a cytoprotective effect through NRF2.

By using CRISPR/Cas9, it is possible to target and knock out the mutated NRF2 protein causing chemoresistance, while not disrupting the function of wildtype NRF2 protein (PCT/US2020/034369, incorporated herein by reference in its entirety). Thus, some embodiments are directed to reducing or, in some embodiments, eliminating expression of variant NRF2s found only in cancer cells and not in non-cancerous cells. These variants are commonly found within the Neh2 Domain of NRF2, which is known as the KEAP1 binding domain. In some embodiments, the NRF2 mutations can be those found in Table 1 below.

TABLE 1

| | | | Notes/Amino |
|---|---|---|---|
| | | | Acid substitution |
| SEQ | | Base | (relative to |
| ID | Sequence, with substitution | position in | position in SEQ |
| NO: | identified in brackets | SEQ ID NO: 15 | ID NO: 8) |
| 1 | TTTGATTGACATACTTTGGAGGC(C > G) | 205-227 | Q26E |
| 2 | CTTACTCCAAGATCTATATCTT(T > G)G | 249-227 | Reverse |
| | | | complement; |
| | | | Q26P |
| 3 | ATACTTTGGAGGCAAGATATAGA(A > G) | 215-237 | D29G |
| 4 | AGGCAAGATATAGATCTTGGAGT(T > G) | 224-246 | V32G |
| 5 | GATATAGATCTTGGAGTAAGTC(C > G)G | 230-252 | R34G |
| 6 | TGACTGAAGTCAAATACTTCTC(C > G)G | 273-251 | Reverse |
| | | | complement; |
| | | | R34P |
| 7 | TTCATCTAGTTGTAACTGAGCGA(A > G) | 385-363 | Reverse |
| | | | complement; |
| | | | F71S |
| 8 | ATTCACCTGTCTCTTCATCTAGT(T > G) | 398-376 | Reverse |
| | | | complement; |
| | | | Q75H |
| 9 | CTCAGTTACAACTAGATGAAGA(A > G)G | 366-388 | E79G |
| 10 | TGAATTGGGAGAAATTCACCTGT(T > G) | 411-389 | Reverse |
| | | | complement; |
| | | | T80P |
| 11 | CAACTAGATGAAGAGACAGGTGA(A > G) | 374-396 | E82W |
| 12 | ATAATAGCTCCTCCCAAACTTGC(C > G) | 728-706 | Reverse |
| | | | complement; |
| | | | E185D |
| 13 | TTTTTCGCTCAGTTACAACTAGA(A > G) | 360-381 | D77G |
| 14 | CAACTAGATGAAGAGACAGGTGA(A > G) | 374-396 | E82G |

PAM sequence underlined.

| NRF2 SEQ ID NO: 15: | |
|---|---|
| 1 | gattaccgag tgccggggag cccggaggag ccgccgacgc agccgccacc gccgccgccg |
| 61 | ccgccaccag agccgccctg tccgcgccgc gcctcggcag ccggaacagg gccgccgtcg |
| 121 | gggagcccca acacacggtc cacagctcat catgatggac ttggagctgc cgccgccggg |
| 181 | actcccgtcc cagcaggaca tggatttgat tgacatactt tggaggcaag atatagatct |
| 241 | tggagtaagt cgagaagtat ttgacttcag tcagcgacgg aaagagtatg agctggaaaa |
| 301 | acagaaaaaa cttgaaaagg aaagacaaga acaactccaa aaggagcaag agaaagcctt |
| 361 | tttcgctcag ttacaactag atgaagagac aggtgaattt ctcccaattc agccagccca |
| 421 | gcacatccag tcagaaacca gtggatctgc caactactcc caggttgccc acattcccaa |
| 481 | atcagatgct ttgtactttg atgactgcat gcagcttttg gcgcagacat tcccgtttgt |
| 541 | agatgacaat gaggtttctt cggctacgtt tcagtcactt gttcctgata ttcccggtca |
| 601 | catcgagagc ccagtcttca ttgctactaa tcaggctcag tcacctgaaa cttctgttgc |
| 661 | tcaggtagcc cctgttgatt tagacggtat gcaacaggac attgagcaag tttgggagga |
| 721 | gctattatcc attcctgagt tacagtgtct taatattgaa aatgacaagc tggttgagac |
| 781 | taccatggtt ccaagtccag aagccaaact gacagaagtt gacaattatc atttttactc |
| 841 | atctataccc tcaatggaaa aagaagtagg taactgtagt ccacattttc ttaatgcttt |
| 901 | tgaggattcc ttcagcagca tcctctccac agaagacccc aaccagttga cagtgaactc |
| 961 | attaaattca gatgccacag tcaacacaga ttttggtgat gaattttatt ctgctttcat |
| 1021 | agctgagccc agtatcagca acagcatgcc ctcacctgct actttaagcc attcactctc |
| 1081 | tgaacttcta aatgggccca ttgatgtttc tgatctatca ctttgcaaag ctttcaacca |
| 1141 | aaaccaccct gaaagcacag cagaattcaa tgattctgac tccggcattt cactaaacac |
| 1201 | aagtcccagt gtggcatcac cagaacactc agtggaatct tccagctatg gagacacact |
| 1261 | acttggcctc agtgattctg aagtggaaga gctagatagt gcccctggaa gtgtcaaaca |
| 1321 | gaatggtcct aaaacaccag tacattcttc tggggatatg gtacaaccct tgtcaccatc |
| 1381 | tcaggggcag agcactcacg tgcatgatgc ccaatgtgag aacacaccag agaaagaatt |
| 1441 | gcctgtaagt cctggtcatc ggaaaacccc attcacaaaa gacaaacatt caagccgctt |
| 1501 | ggaggctcat ctcacaagag atgaacttag ggcaaaagct ctccatatcc cattccctgt |
| 1561 | agaaaaaatc attaacctcc ctgttgttga cttcaacgaa atgatgtcca aagagcagtt |
| 1621 | caatgaagct caacttgcat taattcggga tatacgtagg aggggtaaga ataaagtggc |
| 1681 | tgctcagaat tgcagaaaaa gaaaactgga aaatatagta gaactagagc aagatttaga |
| 1741 | tcatttgaaa gatgaaaaag aaaaattgct caaagaaaaa ggagaaaatg acaaaagcct |
| 1801 | tcacctactg aaaaaacaac tcagcacctt atatctcgaa gttttcagca tgctacgtga |
| 1861 | tgaagatgga aaaccttatt ctcctagtga atactccctg cagcaaacaa gagatggcaa |
| 1921 | tgttttcctt gttcccaaaa gtaagaagcc agatgttaag aaaaactaga tttaggagga |
| 1981 | tttgaccttt tctgagctag tttttttgta ctattatact aaaagctcct actgtgatgt |
| 2041 | gaaatgctca tactttataa gtaattctat gcaaaatcat agccaaaact agtatagaaa |
| 2101 | ataatacgaa actttaaaaa gcattggagt gtcagtatgt tgaatcagta gtttcacttt |
| 2161 | aactgtaaac aatttcttag gacaccattt gggctagttt ctgtgtaagt gtaaatacta |
| 2221 | caaaaactta tttatactgt tcttatgtca tttgttatat tcatagattt atatgatgat |
| 2281 | atgacatctg gctaaaaaga aattattgca aaactaacca ctatgtactt ttttataaat |
| 2341 | actgtatgga caaaaaatgg cattttttat attaaattgt ttagctctgg caaaaaaaaa |
| 2401 | aaattttaag agctggtact aataaaggat tattatgact gttaaa |

In some embodiments, the cancer gene is epidermal growth factor receptor (EGFR). EGFR is a transmembrane glycoprotein that is a member of the protein kinase superfamily. This protein is a receptor for members of the epidermal growth factor family. EGFR is a cell surface protein that binds to epidermal growth factor, thus inducing receptor dimerization and tyrosine autophosphorylation leading to cell proliferation. Mutations in this gene are associated with lung cancer. EGFR is a component of the cytokine storm which contributes to a severe form of COVID-19 resulting from infection with severe acute respiratory syndrome coronavirus-2 (SARS-CoV-2). In some embodiments, a de novo PAM (CTG→CGG) is created in EGFR by a L858R mutation (highlighted in SEQ ID NOs: 16 and 17 below).

| SEQ ID NO: 16 | |
|---|---|
| 1 | agacgtccgg gcagcccccg gcgcagcgcg gccgcagcag cctccgcccc ccgcacggtg |
| 61 | tgagcgcccg acgcggccga ggcggccgga gtcccgagct agccccggcg gccgccgccg |
| 121 | cccagaccgg acgacaggcc acctcgtcgg cgtccgcccg agtccccgcc tcgccgccaa |
| 181 | cgccacaacc accgcgcacg gccccctgac tccgtccagt attgatcggg agagccggag |
| 241 | cgagctcttc ggggagcagc gatgcgaccc tccgggacgg ccggggcagc gctcctggcg |
| 301 | ctgctggctg cgctctgccc ggcgagtcgg gctctggagg aaaagaaagt ttgccaaggc |
| 361 | acgagtaaca agctcacgca gttgggcact tttgaagatc attttctcag cctccagagg |
| 421 | atgttcaata actgtgaggt ggtccttggg aatttggaaa ttacctatgt gcagaggaat |
| 481 | tatgatcttt ccttcttaaa gaccatccag gaggtggctg gttatgtcct cattgccctc |
| 541 | aacacagtgg agcgaattcc tttggaaaac ctgcagatca tcagaggaaa tatgtactac |
| 601 | gaaaattcct atgccttagc agtcttatct aactatgatg caaataaaac cggactgaag |
| 661 | gagctgccca tgagaaattt acaggaaatc ctgcatggcg ccgtgcggtt cagcaacaac |
| 721 | cctgccctgt gcaacgtgga gagcatccag tggcgggaca tagtcagcag tgactttctc |
| 781 | agcaacatgt cgatggactt ccagaaccac ctgggcagct gccaaaagtg tgatccaagc |
| 841 | tgtcccaatg ggagctgctg gggtgcagga gaggagaact gccagaaact gaccaaaatc |
| 901 | atctgtgccc agcagtgctc cgggcgctgc cgtggcaagt cccccagtga ctgctgccac |
| 961 | aaccagtgtg ctgcaggctg cacaggcccc cgggagagcg actgcctggt ctgccgcaaa |
| 1021 | ttccgagacg aagccacgtg caaggacacc tgccccccac tcatgctcta caaccccacc |
| 1081 | acgtaccaga tggatgtgaa ccccgagggc aaatacagct ttggtgccac ctgcgtgaag |
| 1141 | aagtgtcccc gtaattatgt ggtgacagat cacggctcgt gcgtccgagc ctgtggggcc |
| 1201 | gacagctatg agatggagga agacggcgtc cgcaagtgta agaagtgcga agggccttgc |
| 1261 | cgcaaagtgt gtaacggaat aggtattggt gaatttaaag actcactctc cataaatgct |
| 1321 | acgaatatta aacacttcaa aaactgcacc tccatcagtg gcgatctcca catcctgccg |
| 1381 | gtggcattta ggggtgactc cttcacacat actcctcctc tggatccaca ggaactggat |
| 1441 | attctgaaaa ccgtaaagga aatcacaggg tttttgctga ttcaggcttg gcctgaaaac |
| 1501 | aggacggacc tccatgcctt tgagaaccta gaaatcatac gcggcaggac caagcaacat |
| 1561 | ggtcagtttt ctcttgcagt cgtcagcctg aacataacat ccttgggatt acgctccctc |
| 1621 | aaggagataa gtgatggaga tgtgataatt tcaggaaaca aaaatttgtg ctatgcaaat |
| 1681 | acaataaact ggaaaaaact gtttgggacc tccggtcaga aaaccaaaat tataagcaac |
| 1741 | agaggtgaaa acagctgcaa ggccacaggc caggtctgcc atgccttgtg ctcccccgag |
| 1801 | ggctgctggg gcccggagcc cagggactgc gtctcttgcc ggaatgtcag ccgaggcagg |
| 1861 | gaatgcgtgg acaagtgcaa ccttctggag ggtgagccaa gggagtttgt ggagaactct |
| 1921 | gagtgcatac agtgccaccc agagtgcctg cctcaggcca tgaacatcac ctgcacagga |
| 1981 | cggggaccag acaactgtat ccagtgtgcc cactacattg acggccccca ctgcgtcaag |
| 2041 | acctgcccgg caggagtcat gggagaaaac aacaccctgg tctggaagta cgcagacgcc |
| 2101 | ggccatgtgt gccacctgtg ccatccaaac tgcacctacg gatgcactgg gccaggtctt |
| 2161 | gaaggctgtc caacgaatgg gcctaagatc ccgtccatcg ccactgggat ggtgggggcc |
| 2221 | ctcctcttgc tgctggtggt ggccctgggg atcggcctct tcatgcgaag gcgccacatc |
| 2281 | gttcggaagc gcacgctgcg gaggctgctg caggagaggg agcttgtgga gcctcttaca |
| 2341 | cccagtggag aagctcccaa ccaagctctc ttgaggatct tgaaggaaac tgaattcaaa |
| 2401 | aagatcaaag tgctgggctc cggtgcgttc ggcacggtgt ataagggact ctggatccca |
| 2461 | gaaggtgaga aagttaaaat tcccgtcgct atcaaggaat taagagaagc aacatctccg |
| 2521 | aaagccaaca aggaaatcct cgatgaagcc tacgtgatgg ccagcgtgga caacccccac |
| 2581 | gtgtgccgcc tgctgggcat ctgcctcacc tccaccgtgc agctcatcac gcagctcatg |
| 2641 | cccttcggct gcctcctgga ctatgtccgg gaacacaaag acaatattgg ctcccagtac |
| 2701 | ctgctcaact ggtgtgtgca gatcgcaaag ggcatgaact acttggagga ccgtcgcttg |
| 2761 | gtgcaccgcg acctggcagc caggaacgta ctggtgaaaa caccgcagca tgtcaagatc |
| 2821 | acagattttg ggctggccaa actgctgggt gcggaagaga aagaatacca tgcagaagga |
| 2881 | ggcaaagtgc ctatcaagtg gatggcattg gaatcaattt tacacagaat ctatacccac |
| 2941 | cagagtgatg tctggagcta cggggtgact gtttgggagt tgatgacctt tggatccaag |
| 3001 | ccatatgacg gaatccctgc cagcgagatc tcctccatcc tggagaaagg agaacgcctc |
| 3061 | cctcagccac ccatatgtac catcgatgtc tacatgatca tggtcaagtg ctggatgata |
| 3121 | gacgcagata gtcgcccaaa gttccgtgag ttgatcatcg aattctccaa aatggcccga |
| 3181 | gacccccagc gctaccttgt cattcagggg gatgaaagaa tgcatttgcc aagtcctaca |
| 3241 | gactccaact tctaccgtgc cctgatggat gaagaagaca tggacgacgt ggtggatgcc |
| 3301 | gacgagtacc tcatcccaca gcagggcttc ttcagcagcc cctccacgtc acggactccc |
| 3361 | ctcctgagct ctctgagtgc aaccagcaac aattccaccg tggcttgcat tgatagaaat |
| 3421 | gggctgcaaa gctgtcccat caaggaagac agcttcttgc agcgatacag ctcagacccc |
| 3481 | acaggcgcct tgactgagga cagcatagac gacaccttcc tcccagtgcc tgaatacata |
| 3541 | aaccagtccg ttcccaaaag gcccgctggc tctgtgcaga atcctgtcta tcacaatcag |
| 3601 | cctctgaacc ccgcgcccag cagagaccca cactaccagg acccccacag cactgcagtg |
| 3661 | ggcaaccccg agtatctcaa cactgtccag cccacctgtg tcaacagcac attcgacagc |
| 3721 | cctgcccact gggcccagaa aggcagccac caaattagcc tggacaaccc tgactaccag |
| 3781 | caggacttct ttcccaagga agccaagcca aatggcatct ttaagggctc cacagctgaa |
| 3841 | aatgcagaat acctaagggt cgcgccacaa agcagtgaat ttattggagc atgaccacgg |
| 3901 | aggatagtat gagccctaaa aatccagact ctttcgatac ccaggaccaa gccacagcag |
| 3961 | gtcctccatc ccaacagcca tgcccgcatt agctcttaga cccacagact ggttttgcaa |
| 4021 | cgtttacacc gactagccag gaagtacttc cacctcgggc acattttggg aagttgcatt |
| 4081 | cctttgtctt caaactgtga agcatttaca gaaacgcatc cagcaagaat attgtccctt |
| 4141 | tgagcagaaa tttatctttc aaagaggtat atttgaaaaa aaaaaaaagt atatgtgagg |
| 4201 | atttttattg attggggatc ttggagtttt tcattgtcgc tattgatttt tacttcaatg |
| 4261 | ggctcttcca acaaggaaga agcttgctgg tagcacttgc taccctgagt tcatccaggc |
| 4321 | ccaactgtga gcaaggagca caagccacaa gtcttccaga ggatgcttga ttccagtggt |
| 4381 | tctgcttcaa ggcttccact gcaaaacact aaagatccaa gaaggccttc atggccccag |
| 4441 | caggccggat cggtactgta tcaagtcatg gcaggtacag taggataagc cactctgtcc |
| 4501 | cttcctgggc aaagaagaaa cggaggggat ggaattcttc cttagactta cttttgtaaa |
| 4561 | aatgtcccca cggtacttac tccccactga tggaccagtg gtttccagtc atgagcgtta |
| 4621 | gactgacttg tttgtcttcc attccattgt tttgaaactc agtatgctgc ccctgtcttg |
| 4681 | ctgtcatgaa atcagcaaga gaggatgaca catcaaataa taactcggat tccagcccac |
| 4741 | attggattca tcagcatttg gaccaatagc ccacagctga gaatgtggaa tacctaagga |
| 4801 | tagcaccgct tttcttctcg caaaaacgta tctcctaatt tgaggctcag atgaaatgca |
| 4861 | tcaggtcctt tggggcatag atcagaagac tacaaaaatg aagctgctct gaaatctcct |
| 4921 | ttagccatca ccccaacccc ccaaaattag tttgtgttac ttatggaaga tagttttctc |
| 4981 | cttttacttc acttcaaaag ctttttactc aaagagtata tgttccctcc aggtcagctg |
| 5041 | cccccaaacc ccctccttac gctttgtcac acaaaaagtg tctctgcctt gagtcatcta |
| 5101 | ttcaagcact tacagctctg gccacaacag ggcattttac aggtgcgaat gacagtagca |
| 5161 | ttatgagtag tgtggaattc aggtagtaaa tatgaaacta gggtttgaaa ttgataatgc |
| 5221 | tttcacaaca tttgcagatg ttttagaagg aaaaaagttc cttcctaaaa taatttctct |
| 5281 | acaattggaa gattggaaga ttcagctagt taggagccca ccttttttcc taatctgtgt |
| 5341 | gtgccctgta acctgactgg ttaacagcag tcctttgtaa acagtgtttt aaactctcct |
| 5401 | agtcaatatc caccccatcc aatttatcaa ggaagaaatg gttcagaaaa tattttcagc |
| 5461 | ctacagttat gttcagtcac acacacatac aaaatgttcc ttttgctttt aaagtaattt |
| 5521 | ttgactccca gatcagtcag agcccctaca gcattgttaa gaaagtattt gatttttgtc |
| 5581 | tcaatgaaaa taaaactata ttcatttcca ctctattatg ctctcaaata cccctaagca |
| 5641 | tctatactag cctggtatgg gtatgaaaga tacaaagata aataaaacat agtccctgat |
| 5701 | tctaagaaat tcacaattta gcaaaggaaa tggactcata gatgctaacc ttaaaacaac |
| 5761 | gtgacaaatg ccagacagga cccatcagcc aggcactgtg agagcacaga gcagggaggt |
| 5821 | tgggtcctgc ctgaggagac ctggaaggga ggcctcacag gaggatgacc aggtctcagt |
| 5881 | cagcggggag gtggaaagtg caggtgcatc aggggcaccc tgaccgagga aacagctgcc |
| 5941 | agaggcctcc actgctaaag tccacataag gctgaggtca gtcaccctaa acaacctgct |
| 6001 | ccctctaagc caggggatga gcttggagca tcccacaagt tccctaaaag ttgcagcccc |
| 6061 | cagggggatt ttgagctatc atctctgcac atgcttagtg agaagactac acaacatttc |
| 6121 | taagaatctg agattttata ttgtcagtta accactttca ttattcattc acctcaggac |
| 6181 | atgcagaaat atttcagtca gaactgggaa acagaaggac ctacattctg ctgtcactta |
| 6241 | tgtgtcaaga agcagatgat cgatgaggca ggtcagttgt aagtgagtca cattgtagca |
| 6301 | ttaaattcta gtatttttgt agtttgaaac agtaacttaa taaaagagca aaagctattc |
| 6361 | tagctttctt cttcatattt taattttcca ccataaagtt tagttgctaa attctattaa |
| 6421 | ttttaagatt gtgcttccca aaatagttct cacttcatct gtccagggag gcacagttct |
| 6481 | gtctggtaga agccgcaaag cccttagcct cttcacggat ctggcgactg tgatgggcag |
| 6541 | gtcaggagag gagctgccca aagtcccatg attttcacct aacagccctg atcagtcagt |
| 6601 | actcaaagct tggactccat ccctgaaggt cttcctgatt gatagcctgg ccttaatacc |
| 6661 | ctacagaaag cctgtccatt ggctgtttct tcctcagtca gttcctggaa gaccttaccc |
| 6721 | catgacccca gcttcagatg tggtctttgg aaacagaggt cgaaggaaag taaggagctg |
| 6781 | agagctcaca ttcataggtg ccgccagcct tcgtgcatct tcttgcatca tctctaagga |
| 6841 | gctcctctaa ttacaccatg cccgtcaccc catgagggat cagagaaggg atgagtcttc |
| 6901 | taaactctat attcgctgtg agtccaggtt gtaaggggga gcactgtgga tgcatcctat |
| 6961 | tgcactccag ctgatgacac caaagcttag gtgtttgctg aaagttcttg atgttgtgac |
| 7021 | ttaccacccc tgcctcacaa ctgcagacat aaggggacta tggattgctt agcaggaaag |
| 7081 | gcactggttc tcaagggcgg ctgcccttgg gaatcttctg gtcccaacca gaaagactgt |
| 7141 | ggcttgattt tctcaggtgc agcccagccg tagggccttt tcagagcacc ccctggttat |
| 7201 | tgcaacattc atcaaagttt ctagaacctc tggcctaaag gaagggcctg gtgggatcta |
| 7261 | cttggcactc gctggggggc caccccccag tgccactctc actaggcctc tgattgcact |
| 7321 | tgtgtaggat gaagctggtg ggtgatggga actcagcacc tcccctcagg cagaaaagaa |
| 7381 | tcatctgtgg agcttcaaaa gaaggggcct ggagtctctg cagaccaatt caacccaaat |
| 7441 | ctcgggggct ctttcatgat tctaatgggc aaccagggtt gaaaccctta tttctagggt |
| 7501 | cttcagttgt acaagactgt gggtctgtac cagagccccc gtcagagtag aataaaaggc |
| 7561 | tgggtagggt agagattccc atgtgcagtg gagagaacaa tctgcagtca ctgataagcc |
| 7621 | tgagacttgg ctcatttcaa aagcgttcaa ttcatcctca ccagcagttc agctggaaag |
| 7681 | gggcaaatac ccccacctga gctttgaaaa cgccctggga ccctctgcat tctctaagta |
| 7741 | agttatagaa accagtctct tccctccttt gtgagtgagc tgctattcca cgtaggcaac |
| 7801 | acctgttgaa attgccctca atgtctactc tgcatttctt tcttgtgata agcacacact |
| 7861 | tttattgcaa cataatgatc tgctcacatt tccttgcctg ggggctgtaa aaccttacag |
| 7921 | aacagaaatc cttgcctctt tcaccagcca cacctgccat accaggggta cagctttgta |
| 7981 | ctattgaaga cacagacagg atttttaaat gtaaatctat ttttgtaact ttgttgcggg |
| 8041 | atatagttct ctttatgtag cactgaactt tgtacaatat atttttagaa actcattttt |
| 8101 | ctactaaaac aaacacagtt tactttagag agactgcaat agaatcaaaa tttgaaactg |
| 8161 | aaatctttgt ttaaaagggt taagttgagg caagaggaaa gccctttctc tctcttataa |
| 8221 | aaaggcacaa cctcattggg gagctaagct aggtcattgt catggtgaag aagagaagca |
| 8281 | tcgtttttat atttaggaaa ttttaaaaga tgatggaaag cacatttagc ttggtctgag |
| 8341 | gcaggttctg ttggggcagt gttaatggaa agggctcact gttgttacta ctagaaaaat |
| 8401 | ccagttgcat gccatactct catcatctgc cagtgtaacc ctgtacatgt aagaaaagca |
| 8461 | ataacatagc actttgttgg tttatatata taatgtgact tcaatgcaaa ttttattttt |
| 8521 | atatttacaa ttgatatgca tttaccagta taaactagac atgtctggag agcctaataa |
| 8581 | tgttcagcac actttggtta gttcaccaac agtcttacca agcctgggcc cagccaccct |
| 8641 | agagaagtta ttcagccctg gctgcagtga catcacctga ggagctttta aaagcttgaa |
| 8701 | gcccagctac acctcagacc gattaaacgc aaatctctgg ggctgaaacc caagcattcg |
| 8761 | tagtttttaa agctcctgag gtcattccaa tgtgcggcca aagttgagaa ctactggcct |
| 8821 | agggattagc cacaaggaca tggacttgga ggcaaattct gcaggtgtat gtgattctca |
| 8881 | ggcctagaga gctaagacac aaagacctcc acatctgtcg ctgagagtca agaacctgaa |
| 8941 | cagagtttcc atgaaggttc tccaagcact agaagggaga gtgtctaaac aatggttgaa |
| 9001 | aagcaaagga aatataaaac agacacctct ttccatttcc taaggtttct ctctttatta |
| 9061 | agggtggact agtaataaaa tataatattc ttgctgctta tgcagctgac attgttgccc |
| 9121 | tccctaaagc aaccaagtag cctttatttc ccacagtgaa agaaaacgct ggcctatcag |
| 9181 | ttacattaca aaaggcagat ttcaagagga ttgagtaagt agttggatgg ctttcataaa |
| 9241 | aacaagaatt caagaagagg attcatgctt taagaaacat ttgttataca ttcctcacaa |
| 9301 | attatacctg ggataaaaac tatgtagcag gcagtgtgtt ttccttccat gtctctctgc |
| 9361 | actacctgca gtgtgtcctc tgaggctgca agtctgtcct atctgaattc ccagcagaag |
| 9421 | cactaagaag ctccacccta tcacctagca gataaaacta tggggaaaac ttaaatctgt |
| 9481 | gcatacattt ctggatgcat ttacttatct ttaaaaaaaa aggaatccta tgacctgatt |
| 9541 | tggccacaaa aataatcttg ctgtacaata caatctcttg gaaattaaga gatcctatgg |
| 9601 | atttgatgac tggtattaga ggtgacaatg taaccgatta acaacagaca gcaataactt |
| 9661 | cgttttagaa acattcaagc aatagcttta tagcttcaac atatggtacg ttttaacctt |
| 9721 | gaaagttttg caatgatgaa agcagtattt gtacaaatga aaagcagaat tctcttttat |
| 9781 | atggtttata ctgttgatca gaaatgttga ttgtgcattg agtattaaaa aattagatgt |
| 9841 | atattattca ttgttcttta ctcctgagta ccttataata ataataatgt attctttgtt |
| 9901 | aacaa |

SEQ ID NO: 17
MRPSGTAGAA LLALLAALCP ASRALEEKKV CQGTSNKLTQ LGTFEDHELS LQRMENNCEV
VLGNLEITYV QRNYDLSFLK TIQEVAGYVL IALNTVERIP LENLQIIRGN MYYENSYALA
VLSNYDANKT GLKELPMRNL QEILHGAVRF SNNPALCNVE SIQWRDIVSS DFLSNMSMDF
QNHLGSCQKC DPSCPNGSCW GAGEENCQKL TKIICAQQCS GRCRGKSPSD CCHNQCAAGC
TGPRESDCLV CRKFRDEATC KDTCPPLMLY NPTTYQMDVN PEGKYSFGAT CVKKCPRNYV
VTDHGSCVRA CGADSYEMEE DGVRKCKKCE GPCRKVCNGI GIGEFKDSLS INATNIKHFK
NCTSISGDLH ILPVAFRGDS FTHTPPLDPQ ELDILKTVKE ITGFLLIQAW PENRTDLHAF
ENLEIIRGRT KQHGQFSLAV VSLNITSLGL RSLKEISDGD VIISGNKNLC YANTINWKKL
FGTSGQKTKI ISNRGENSCK ATGQVCHALC SPEGCWGPEP RDCVSCRNVS RGRECVDKCN
LLEGEPREFV ENSECIQCHP ECLPQAMNIT CTGRGPDNCI QCAHYIDGPH CVKTCPAGVM
GENNTLVWKY ADAGHVCHLC HPNCTYGCTG PGLEGCPTNG PKIPSIATGM VGALLLLLVV
ALGIGLFMRR RHIVRKRTLR RLLQERELVE PLTPSGEAPN QALLRILKET EFKKIKVLGS
GAFGTVYKGL WIPEGEKVKI PVAIKELREA TSPKANKEIL DEAYVMASVD NPHVCRLLGI
CLTSTVQLIT QLMPFGCLLD YVREHKDNIG SQYLLNWCVQ IAKGMNYLED RRLVHRDLAA
RNVLVKTPQH VKITDFGLAK LLGAEEKEYH AEGGKVPIKW MALESILHRI YTHQSDVWSY
GVTVWELMTF GSKPYDGIPA SEISSILEKG ERLPQPPICT IDVYMIMVKC WMIDADSRPK
FRELIIEFSK MARDPQRYLV IQGDERMHLP SPTDSNFYRA LMDEEDMDDV VDADEYLIPQ
QGFFSSPSTS RTPLLSSLSA TSNNSTVACI DRNGLQSCPI KEDSFLQRYS SDPTGALTED
SIDDTFLPVP EYINQSVPKR PAGSVQNPVY HNQPLNPAPS RDPHYQDPHS TAVGNPEYLN
TVQPTCVNST FDSPAHWAQK GSHQISLDNP DYQQDFFPKE AKPNGIFKGS TAENAEYLRV
APQSSEFIGA

### EXAMPLES

The present disclosure is further defined in the following Examples. It should be understood that these Examples, while indicating exemplary embodiments of the present disclosure, are given by way of illustration only.

### Example 1

### LNP Formulations Tested In Vitro

(a) Lipofectamine CRISPRMAX Cas9 Transfection Reagent (Cat #CMAX0015, ThermoFisher Scientific)
(b) ProdeliverIN CRISPR kit (Cat #PIC60100, OZ Biosciences)

Human lung squamous cell carcinoma NCI-H1703 clone 26-8 and 44-25 cells were derived from NCI-H1703 cells (#CRL-5889) which were purchased from ATCC (Manassas, VA, USA). NCI-H1703 clone 26-8 and 44-25 cells contains R34G mutation (C→G) in the NFE2L2 gene.

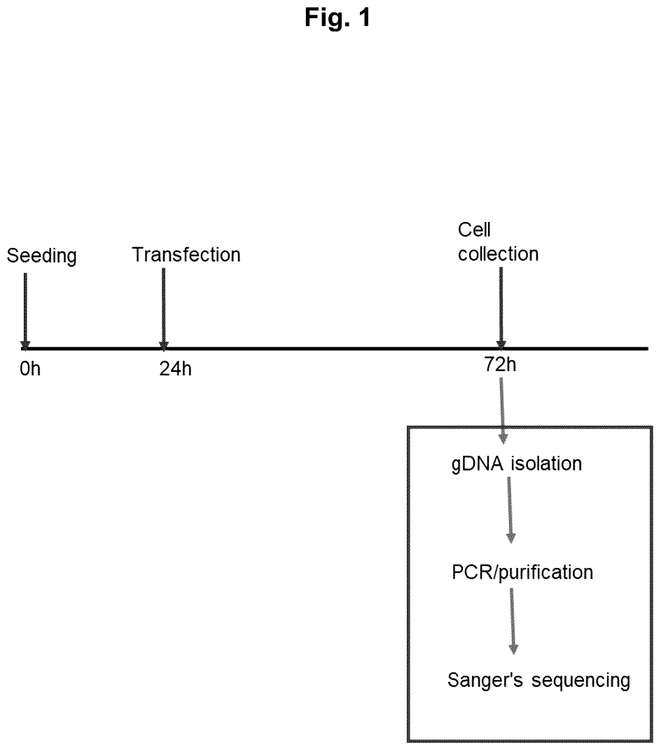

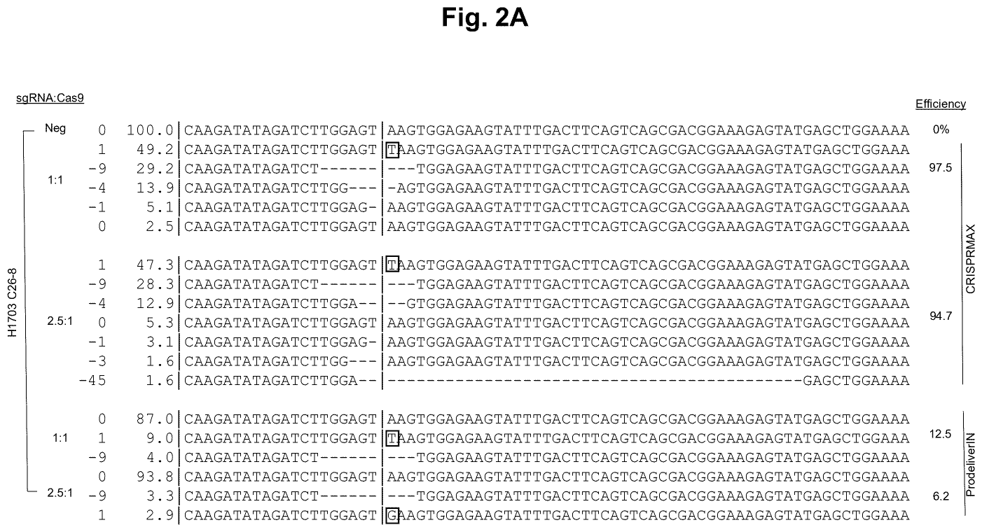

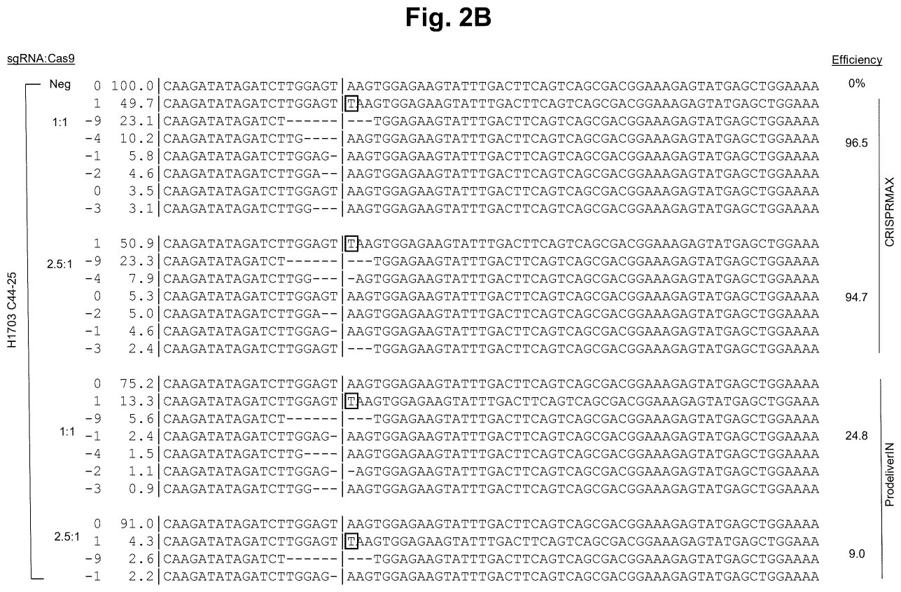

Transfections were performed in the H1703 clonal cell lines (C26-8 and C44-25) utilizing the two above mentioned LNP formulations with NRF2 R34G sgRNA (GATATAGATCTTGGAGTAAG) and spCas9 at varying ratios (as shown in Table 2, FIGS. 1 and 2) per well in 24-well plate format for seeding cell number of 0.4×10⁵ and incubated in a humidified incubator with 5% CO₂ for 48h post transfection.

TABLE 2

| | | | | H1703 C26-8 | | H1703 C44-25 | |
|---|---|---|---|---|---|---|---|
| | | | | | +1 | | +1 |
| LNP for | sgRNA | Cas9 | Molar ratio | % | Indel | % | Indel |
| transfection | (pmol) | (pmol) | (sgRNA:Cas9) | Indel | (%) | Indel | (%) |
| CRISPRMAX | 20 | 20 | 1:1 | 97.5 | 49.2 | 96.5 | 49.7 |
| CRISPRMAX | 50 | 20 | 2.5:1 | 94.7 | 47.3 | 94.7 | 50.9 |
| ProdeliverIN | 20 | 20 | 1:1 | 13 | 9 | 24.8 | 13.3 |
| ProdeliverIN | 50 | 20 | 2.5:1 | 2.9 | 9 | 9.0 | 4.3 |

Summary of findings: Lipofectamine CRISPRMAX Cas9 Transfection Reagent exhibited significantly higher efficiency of editing for each of the tested conditions and was utilized in the next set of studies.

### Example 2

### CRISPR Editing Efficiency of CRISPRMAX Based on Seeding Cell Number

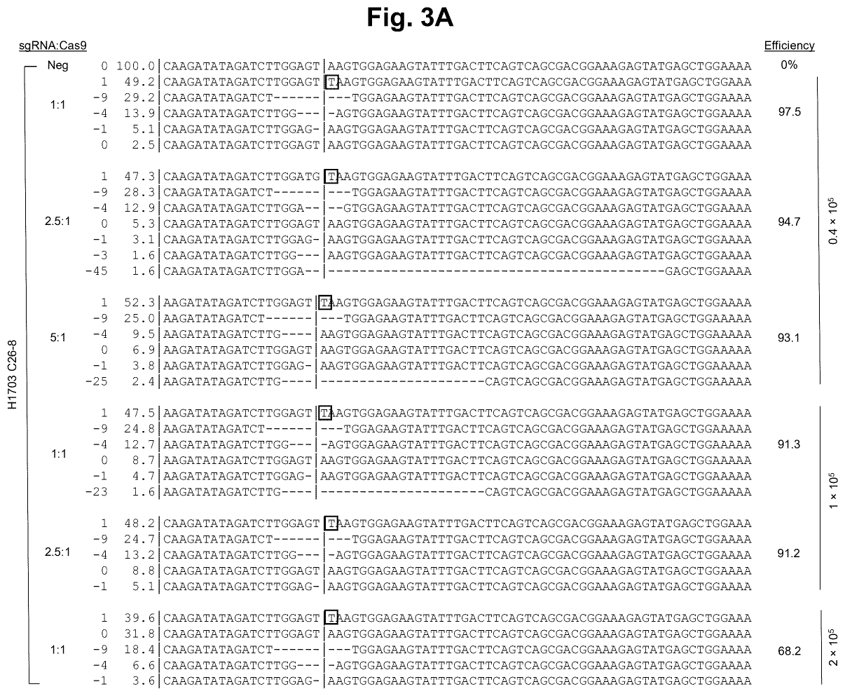

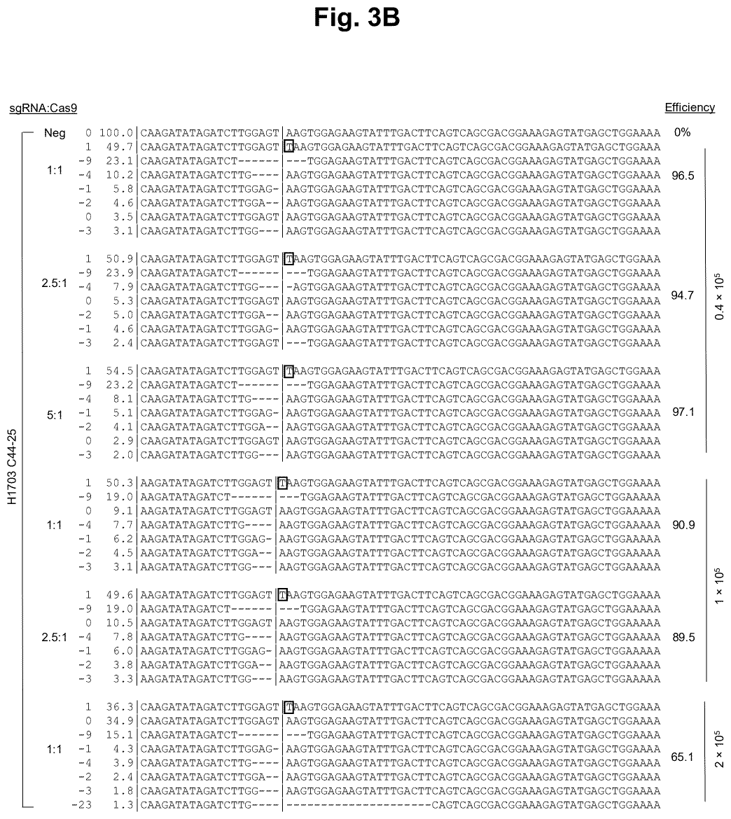

Transfections were performed in the H1703 clonal cell lines (C26-8 and C44-25) utilizing Lipofectamine CRISPRMAX Cas9 Transfection Reagent with NRF2 R34G sgRNA (GATATAGATCTTGGAGTAAG) and spCas9 at varying ratios (as shown in Table 3, FIG. 3) per well in 24-well plate format for three different seeding cell numbers and incubated in a humidified incubator with 5% CO₂ for 48h post transfection.

TABLE 3

| | | | | H1703 C26-8 | | H1703 C44-25 | |
|---|---|---|---|---|---|---|---|
| Seeding | | | | | +1 | | +1 |
| cell | sgRNA | Cas9 | Ratio | % | Indel | % | Indel |
| Number | (pmol) | (pmol) | (sgRNA:Cas9) | Indel | (%) | Indel | (%) |
| 0.4 × 10⁵ | 20 | 20 | 1:1 | 97.5 | 49.2 | 96.5 | 49.7 |
| | 50 | 20 | 2.5:1 | 94.7 | 47.3 | 94.7 | 50.9 |
| | 100 | 20 | 1:1 | 93.1 | 52.3 | 97.1 | 54.5 |
| 1 × 10⁵ | 20 | 20 | 1:1 | 91.3 | 47.6 | 90.9 | 50.6 |
| | 50 | 20 | 2.5:1 | 91.2 | 48.3 | 89.5 | 49.9 |
| 2 × 10⁵ | 20 | 20 | 1:1 | 68.2 | 39.6 | 65.1 | 36.3 |

Summary of findings: Increasing the sgRNA: Cas9 molar ratio did not have significant effect on the gene editing efficiency; however, increase in seeding cell numbers reduced the editing efficiency in the H1703 clonal lines.

### Example 3

Dynamic Analysis for CRISPRMAX Mediated In Vitro Genome Editing Efficiency in H1703 Clonal Cell Lines at 1:1 sgRNA and spCas9 Ratio (20 μMol)

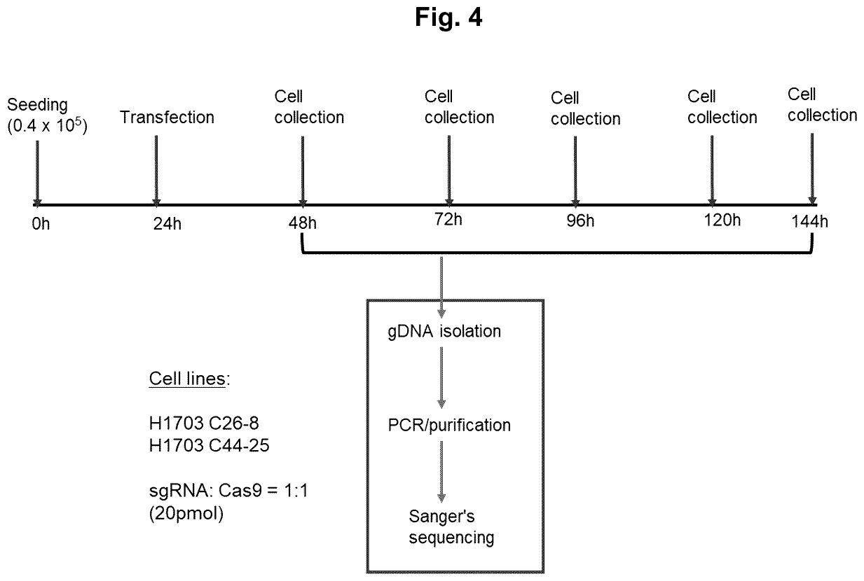

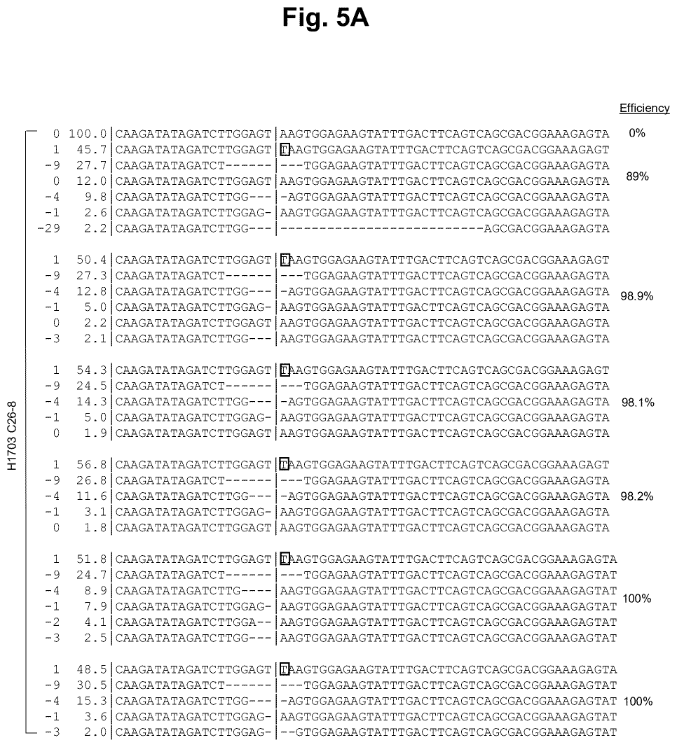

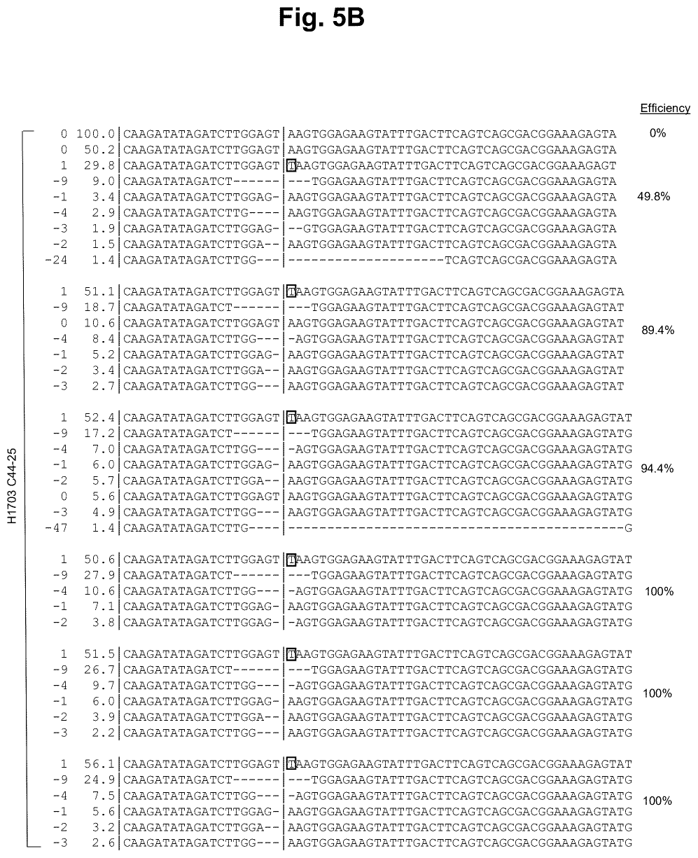

Transfections were performed in the H1703 clonal lines (C26-8 and C44-25) utilizing Lipofectamine CRISPRMAX Cas9 Transfection Reagent with NRF2 R34G sgRNA (GATATAGATCTTGGAGTAAG) and spCas9 at 1:1 ratio (20pmol) per well in 24-well plate format for 0.4×10⁵ seeding cells incubated in a humidified incubator with 5% CO₂. Cells were collected at varied times post transfection (Table 4, FIGS. 4 and 5).

TABLE 4

| | H1703 C26-8 | | | H1703 C44-25 | | |
|---|---|---|---|---|---|---|
| | | +1 | | | +1 | |
| Time post- | % | Indel | Frameshift | % | Indel | Frameshift |
| transfection | Indel | (%) | (%) | Indel | (%) | (%) |
| 24 h | 88 | 45.7 | 60.3 | 49.8 | 29.8 | 37.6 |
| 48 h | 97.8 | 50.4 | 68.5 | 89.4 | 51.1 | 68.1 |
| 72 h | 98.1 | 54.3 | 73.6 | 94.4 | 52.4 | 72.4 |
| 96 h | 98.2 | 56.8 | 71.4 | 100 | 50.6 | 72.1 |
| 120 h | 100 | 51.9 | 71.1 | 100 | 51.5 | 71.1 |
| 144 h | 100 | 56.1 | 67.5 | 100 | 56.1 | 72.4 |

Summary of findings: Significantly increased efficiency in genome editing in both the clonal lines was observed starting at 72 h post-transfection.

Dynamic Analysis for CRISPRMAX Mediated In Vitro Genome Editing Efficiency in H1703 Clonal Cell Lines at 1:1 sgRNA and spCas9 Ratio (10 μMol)

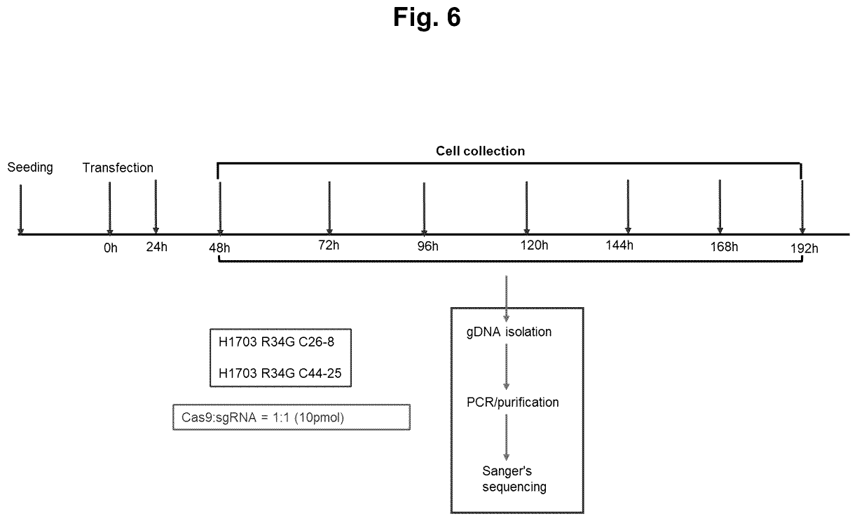

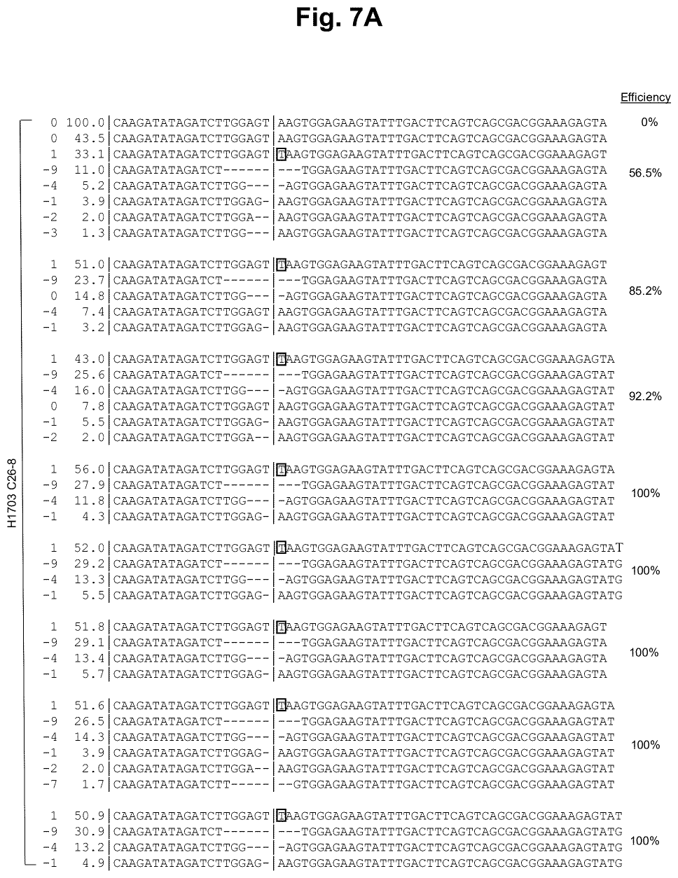

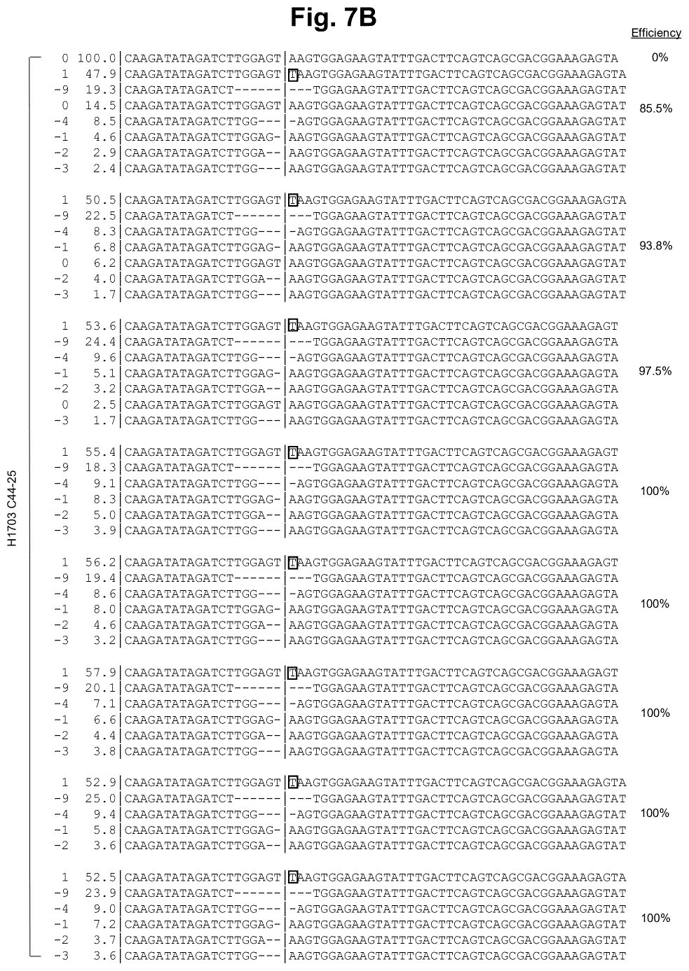

Transfections were performed in the H1703 clonal lines (C26-8 and C44-25) utilizing Lipofectamine CRISPRMAX Cas9 Transfection Reagent with NRF2 R34G sgRNA (GATATAGATCTTGGAGTAAG) and spCas9 at 1:1 ratio (10pmol) per well in 24-well plate format for 0.4×10⁵ seeding cells incubated in a humidified incubator with 5% CO₂. Cells were collected at varied times post transfection (Table 5, FIGS. 6 and 7).

### Example 4

Dynamic Analysis for CRISPRMAX Mediated In Vitro Genome Editing Efficiency in H1703 Clonal Cell Lines at 1:1 sgRNA and spCas9 Ratio (10 μMol)

Transfections were performed in the H1703 clonal lines (C26-8 and C44-25) utilizing Lipofectamine CRISPRMAX Cas9 Transfection Reagent with NRF2 R34G sgRNA (GATATAGATCTTGGAGTAAG) and spCas9 at 1:1 ratio (10pmol) per well in 24-well plate format for 0.4×10⁵ seeding cells incubated in a humidified incubator with 5% CO₂. Cells were collected at varied times post transfection (Table 5, FIGS. 6 and 7).

TABLE 5

| | H1703 C26-8 | | | H1703 C44-25 | | |
|---|---|---|---|---|---|---|
| | | +1 | | | +1 | |
| Time post- | % | Indel | Frameshift | % | Indel | Frameshift |
| transfection | Indel | (%) | (%) | Indel | (%) | (%) |
| 24 h | 56.5 | 33.1 | 44.2 | 85.5 | 47.9 | 63.8 |
| 48 h | 85.2 | 51 | 61.5 | 93.8 | 50.5 | 69.6 |
| 72 h | 92.2 | 43 | 66.5 | 97.5 | 53.6 | 71.4 |
| 96 h | 100 | 56 | 72.1 | 100 | 55.4 | 77.8 |
| 120 h | 100 | 52 | 70.8 | 100 | 56.2 | 77.3 |
| 144 h | 100 | 51.8 | 68.5 | 100 | 57.9 | 76.1 |
| 168 h | 100 | 51.6 | 73.5 | 100 | 52.9 | 71.6 |
| 192 h | 100 | 50.9 | 69.1 | 100 | 52.5 | 72.4 |

Summary of findings: Reduction in the sgRNA Cas9 concentration to 10 pmol from 20 pmol did not alter the editing efficiency of the clonal lines. Efficiency of genome editing was greater than 90% starting at 72 h and was observed to be 100% at later timepoints.

### Example 5

### Chemosensitivity of H1703 WT and Engineered Cell Lines to Cisplatin Post CRISPR-Cas9 Transfection

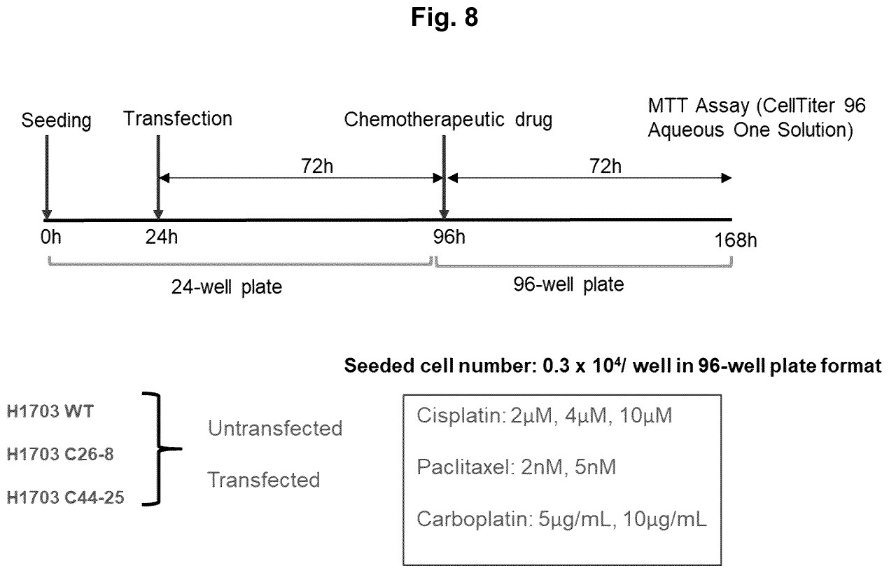

H1703 WT, C26-8 and C44-25 were seeded at 0.4×10⁵ cells per well in 24-well plate format. Cells were transfected with NRF-2 R34G specific sgRNA (GATATAGATCTTGGAGTAAG) or scrambled sgRNA and Cas9 in 1:1 molar ratio at 10 pmol concentration using CRISPRMax reagent. Untransfected or transfected cells were seeded in triplicate at 0.3×10⁴ cells per well in 96-well plate 72 h post-transfection. 24 hrs later, cisplatin (0, 2, 4 and 10 μM), carboplatin (0, 5 and 10 μg/mL), or paclitaxel (0, 2 and 5 nM) were administered to the cells, and cells were incubated for 72 hrs (FIG. 8). Cell viability was quantified by MTT Assay (CellTiter 96 Aqueous One Solution).

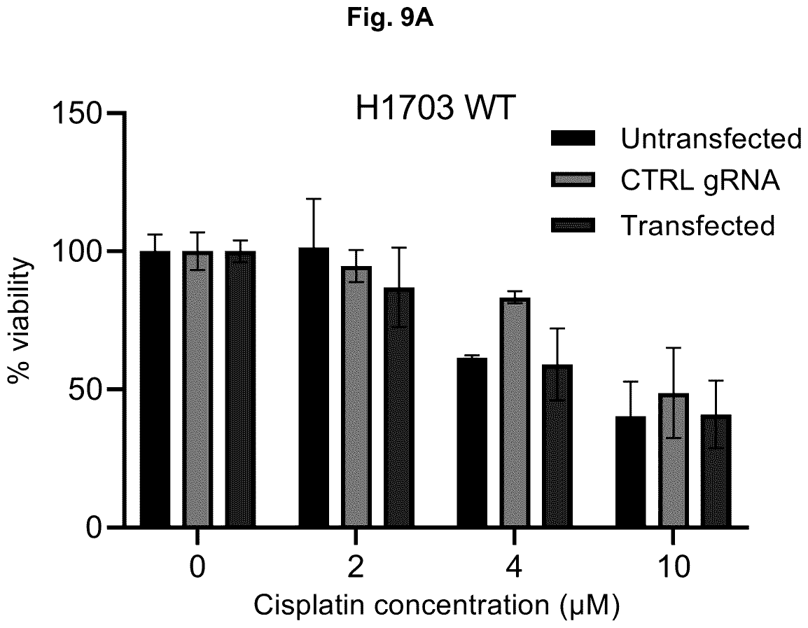

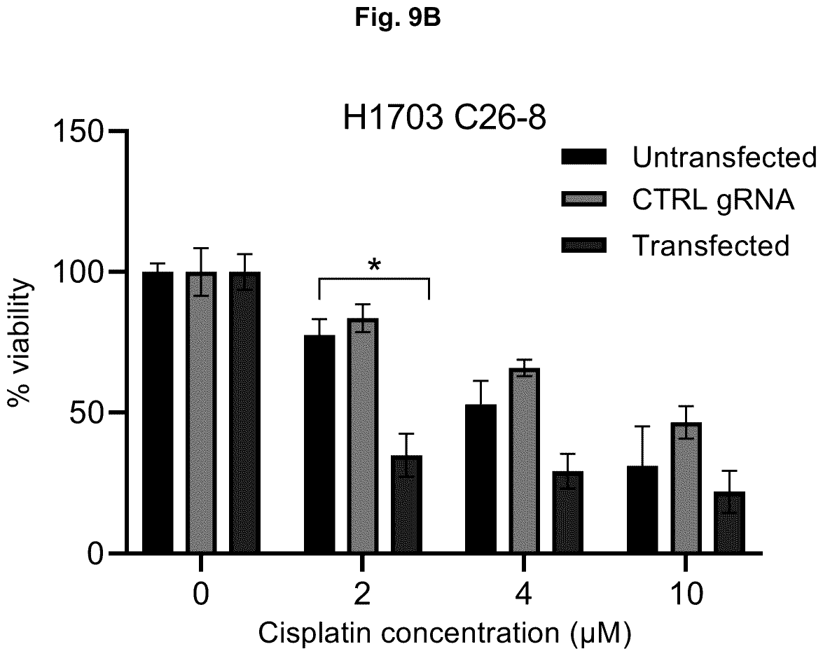

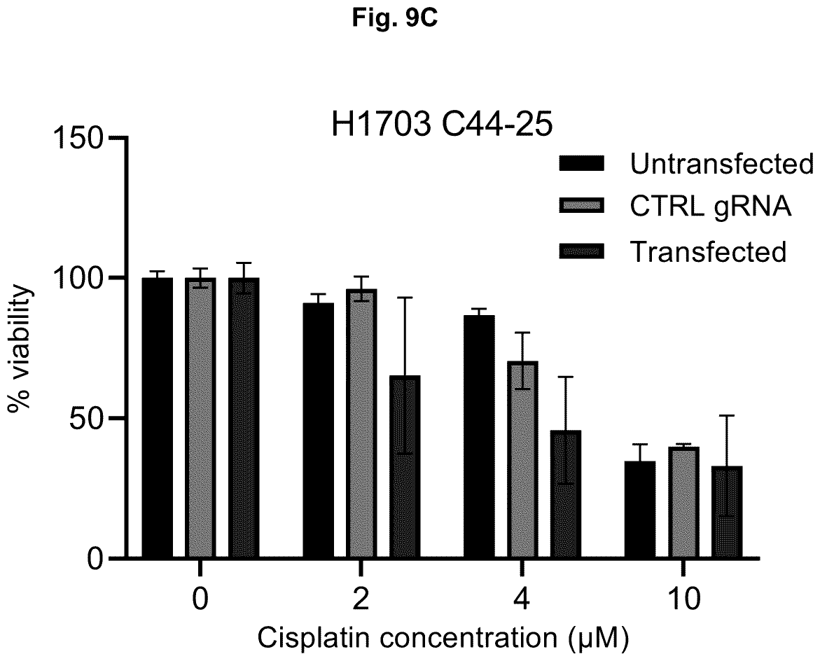

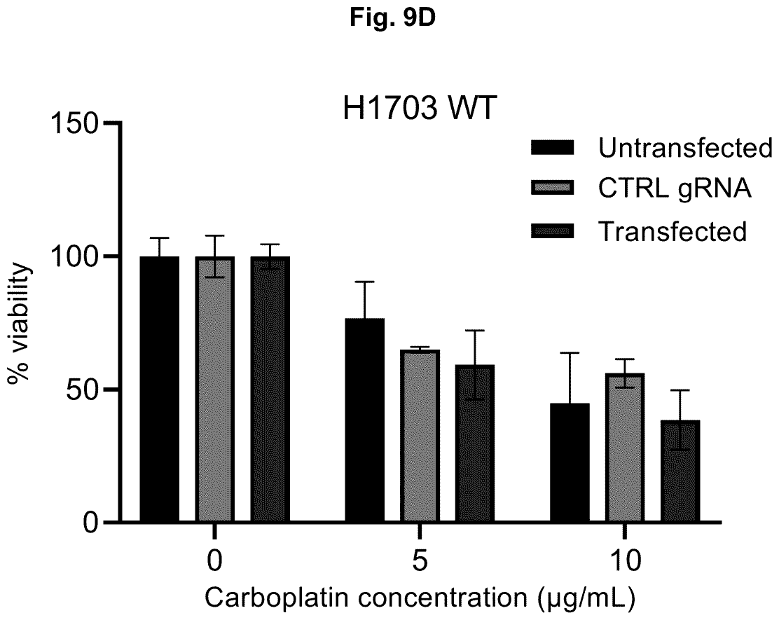

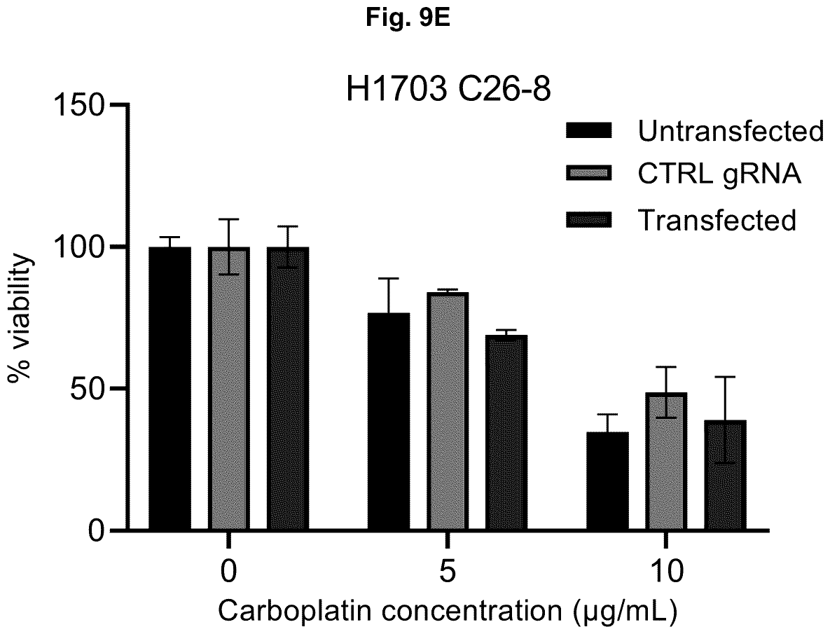

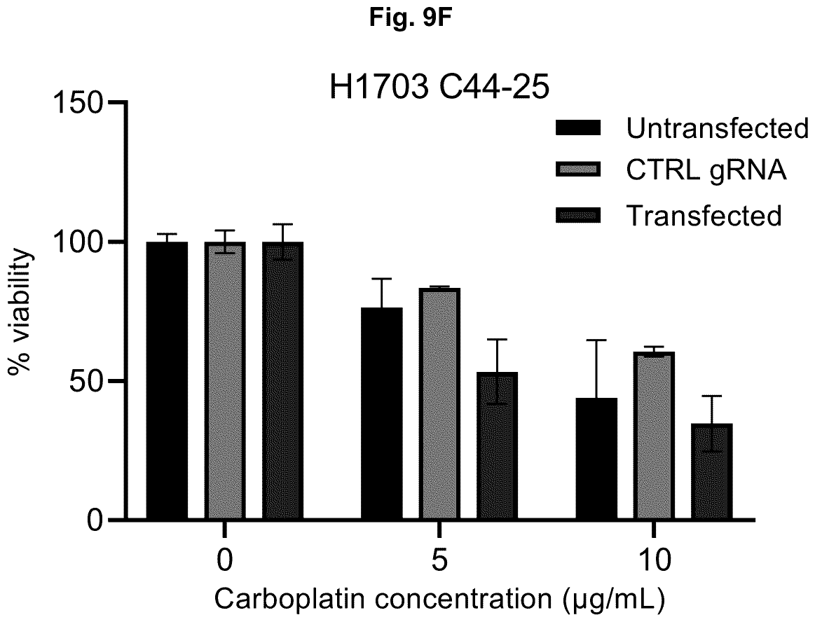

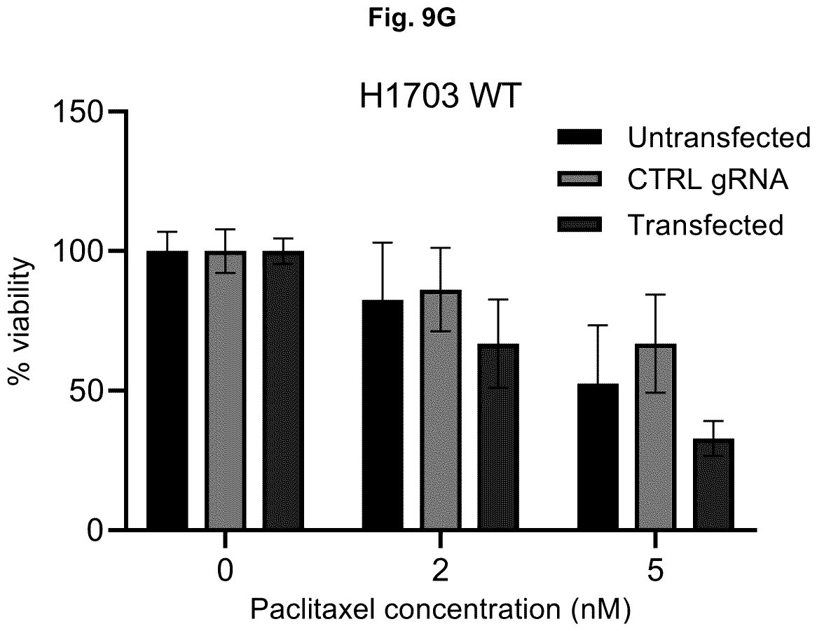

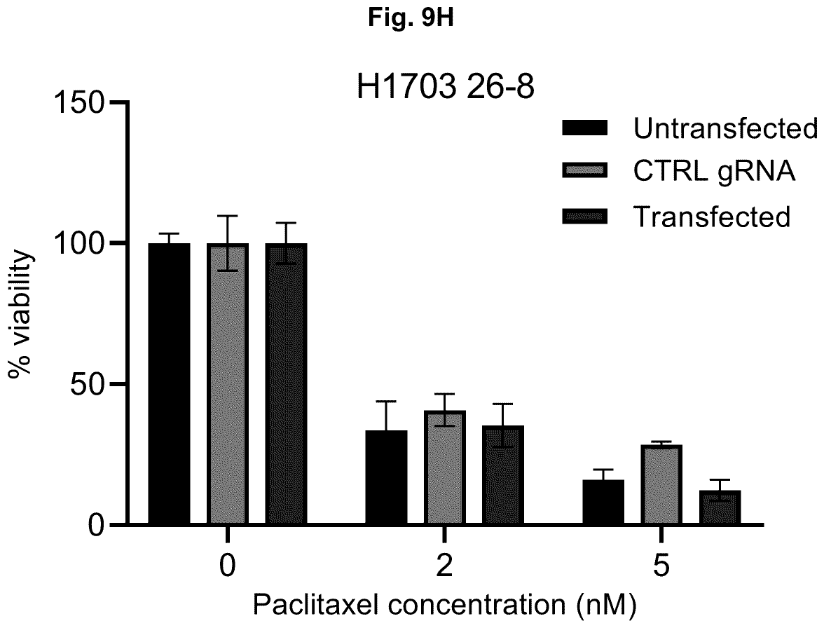

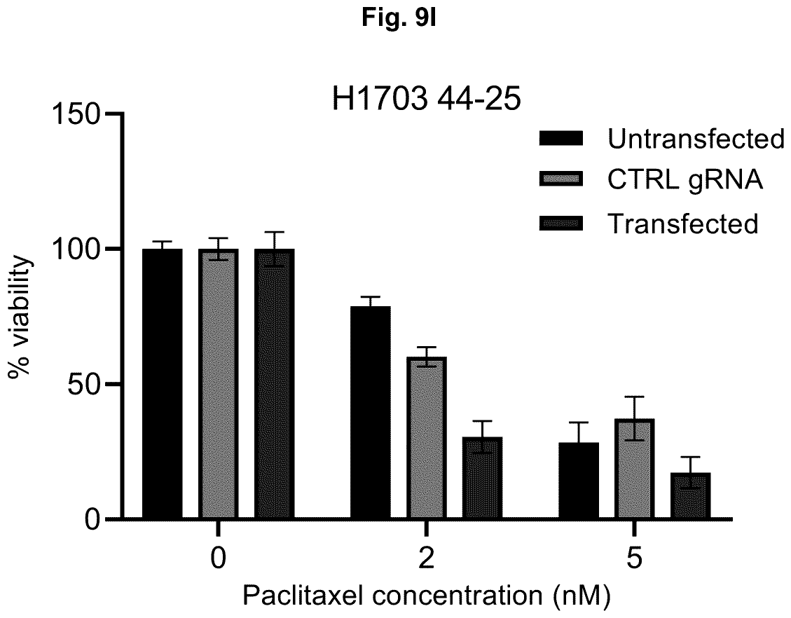

Summary of findings: Significant enhancement in chemosensitivity (p<0.05) was observed for H1703 C26-8 transfected cells as compared to untransfected cells dosed with 2 μM cisplatin, resulting in ˜45% reduction in the number of viable cells in the transfected clonal line (FIG. 9).

### Example 6

### Intratumoral Delivery of LNP/fLuc to H1703 (44-25) SubQ Developed in NCG Mice

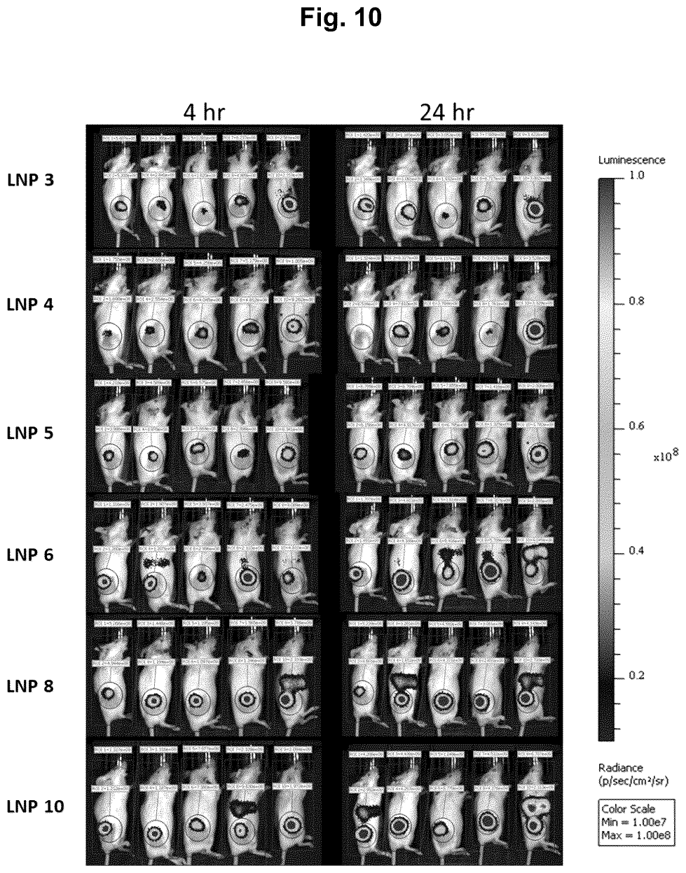

Lipid nanoparticles (LNP) packaged with firefly luciferase mRNA were used to assess intratumoral delivery and expression within a xenograft mouse model. CRISPR-engineered human lung squamous cell carcinoma cell line, H1703 44-25, was implanted subcutaneously. Once tumors reached 60-150 mm³ in size, mice were injected intratumorally with 2 μg of respective LNP. Bioluminescence imaging was conducted on mice 4 and 24 hours post injection. FIG. 10 displays time course of the bioluminescent signal post intratumoral injection. Signal from each tumor is quantified by establishing a region of interest (ROI) of the tumor. The ROI was standardized as one size for all experiments. The ROI value is listed as total flux photons of each tumor. Four hours post intratumoral injection in mice across all 6 LNPs tested created a strong signal with comparable ROIs. The signal increased at 24 hours post injection. The result demonstrates LNP successfully delivers the fLuc mRNA into the cells and allows expression of the fLuc proteins.

### Materials and Methods

### LNP Formulation

Lipid nanoparticles were formulated at Precision Nanosystems using a proprietary library of ionizable lipids and compositions. Firefly luciferase (FLuc) mRNA (CleanCap FLuc mRNA (5moU)) was purchased from TriLink Biotechnologies (San Diego, CA). FLuc-LNPs were prepared by microfluidic mixing of lipid components and aqueous RNA solution. All LNP formulations were filtered and characterized by Precision Nanosystems.

The formulation was to prepare 10 LNP formulations containing 4 different PNI ionizable lipids. fLuc mRNA encapsulated LNPs were prepared using the NanoAssemblr® Ignite™ system with a NxGen cartridge at 12 mL/min (3:1 Aq/Org mixing ratio) using the formulation sceme described in Table 6. Formulations were downstream processed, and buffer exchanged with a PNI proprietary cryopreservation buffer (CB1) using 30 KDa centrifugal filters and characterized for size, PDI, Zeta potential and encapsulation efficiency. The concentration of the fLuc LNP formulations were adjusted to 0.49-0.66 mg/mL, aliquoted in aliquots of 30 μL in 0.5 mL cryo-storage vials and stored at −80° C. and analyzed post one Freeze thaw cycle (Table 7).

TABLE 6
Parameters used for formulation of fLuc mRNA in LNPs

| Formulation | fLuc mRNA LNPs |
|---|---|
| Instrument | NanoAssemblr ™ Ignite ® |
| Cartridge | NxGen |
| Aqueous stream | fLuc mRNA in PNI formulation buffer |
| Organic stream | PNI's lipid mixes in ethanol |
| Flow rate ratio (FRR) | 3:1 |
| (mRNA solution to lipid solution) | |
| Total flow rate (TFR), mL/min | 12 |
| Start waste, mL | 0.95 |
| End waste, mL | 0.05 |

TABLE 7
fLuc mRNA LNP formulations after 1 freeze thaw cycle at −80° C.

| | | | | Zeta | total | Encapsulation |
|---|---|---|---|---|---|---|
| | | Size | | potential | mg/mL | efficiency |
| LNP # | Sample ID | (d · nm) | PDI | (mV) | mRNA | (%) |
| 1 | LSA-iL2V42-Fluc-E02 | 107 | 0.11 | −4.1 | 0.55 | 99 |
| 2 | LSA-iL2V44-Fluc-E02 | 169 | 0.06 | −3.3 | 0.52 | 98 |
| 3 | LSA-iL2V62-Fluc-E02 | 95 | 0.1 | −7.1 | 0.56 | 98 |
| 4 | LSA-iL2V78-Fluc-E02 | 98 | 0.09 | −1.6 | 0.59 | 99 |
| 5 | LSA-iL9V44-Fluc-E02 | 83 | 0.05 | −3.1 | 0.47 | 97 |
| 6 | LSA-iL9V62-Fluc-E02 | 72 | 0.07 | −7.4 | 0.66 | 98 |
| 7 | LSA-iL49V44-Fluc-E02 | 82 | 0.06 | −4.5 | 0.49 | 97 |
| 8 | LSA-iL49V62-Fluc-E02 | 80 | 0.06 | −4.1 | 0.49 | 96 |
| 9 | LSA-iL33V44-Fluc-E02 | 82 | 0.05 | −6.3 | 0.61 | 99 |
| 10 | LSA-iL33V62-Fluc-E02 | 76 | 0.03 | −6.4 | 0.58 | 98 |

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of 5×10⁶ cells (human lung squamous cell-derived H1703 clone 44-25) in BD matirgel (volume ratio 1:1). Tumor growth was measured and estimated using a caliper and calculated as volume (mm³)=(length [mm] x (width [mm]) 2×0.5. When tumors reached ˜60-150 mm³, mice were separated to experimental groups. Mice were intratumorally injected with 2 μg of FLuc-LNP in a total volume of 25 μL (diluted in PBS as needed). Mice were euthanized and tumors were collected at the indicated time points per experiment.

### Bioluminescence Imaging

Bioluminescence imaging was performed with an IVIS Spectrum imaging system (Caliper Life Sciences). Mice were administered intraperitoneally with d-luciferin (Promega) at a dose of 150 mg/kg. Five minutes after receiving d-luciferin, mice were anesthetized in a chamber with 3% isoflurane and placed on the imaging platform while being maintained on 3% isoflurane via a nose cone. Mice were imaged at 15 minutes post administration of d-luciferin using an exposure time of 1 second or longer. Bioluminescence values were quantified by measuring photon flux (photons/second) in the region of interest where bioluminescence signal emanated using the Living IMAGE Software provided by Caliper (Hopkinton, MA).

### Example 7

Quantitative Analysis of fLuc Expression within the Tumors after Intratumoral Delivery of fLuc LNPs

In order to analyze the biodistribution of the luciferase expressing LNPs, quantitative PCR was performed. Briefly, RNA was extracted from tissue samples using the TRIzol RNA extraction protocol as described by the manufacturer (Invitrogen). Complementary DNA (cDNA) was then synthesized using Applied Biosystems High-Capacity RNA-to-cDNA kit at 250 ng per reaction and qPCR was performed in triplicate with Fast SYBR Green Master Mix. Table 8 shows the primer sequences that were used.

TABLE 8
Primer sequences for qPCR analysis. Gapdh-mouse primers were used
for lung, ovary, liver, spleen, heart, blood and brain tissues.
Gapdh-human primers were used for tumor tissues.

| Primer name | Sequence 5′-3′ | SEQ ID NO: |
|---|---|---|
| Gapdh-mouse F | CATGGCCTTCCGTGTTCCTA | 18 |
| Gapdh-mouse R | ACTTGGCAGGTTTCTCCAGG | 19 |
| Gapdh-human F | TCTCCTCTGACTTCAACAGCGAC | 20 |
| Gapdh-human R | CCCTGTTGCTGTAGCCAAATTC | 21 |
| Luciferase LNP-F | TGATGTACCGGTTCGAGGAG | 22 |
| Luciferase LNP-R | TTGTCGATCAGGGTGCTCTT | 23 |

Samples were run on a Bio-Rad CFX384 real time PCR detection system according to the manufactures conditions: 95° for 20 sec followed by 40 cycles of 95° for 3° seconds and 60° for 30 seconds. Relative quantification for each transcript was obtained by normalizing against Gapdh transcript abundance according to the formula 2 ^{(-Ct)}/2 ^{(CtGapdh)}.

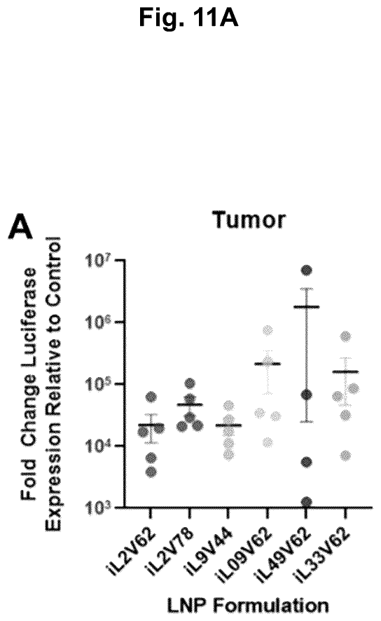

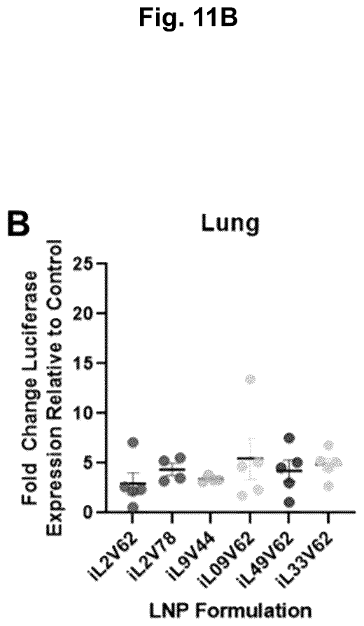

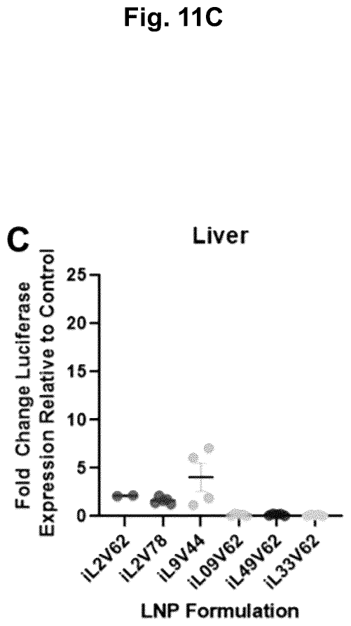

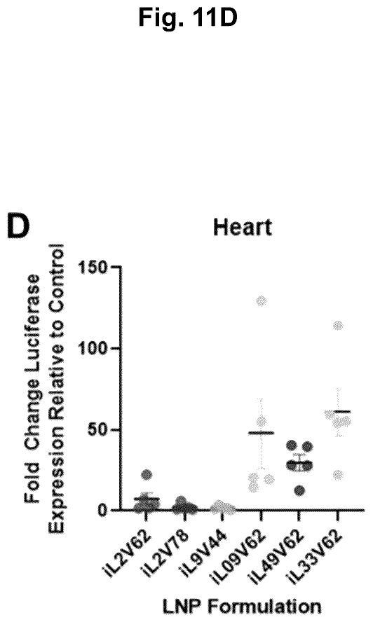

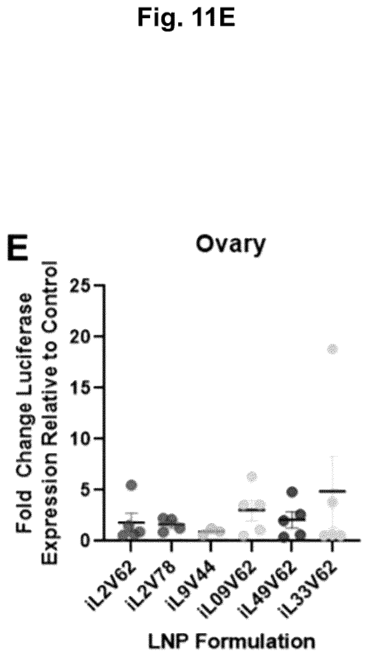

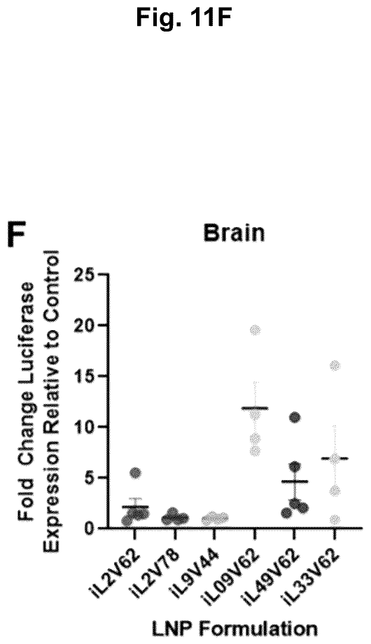

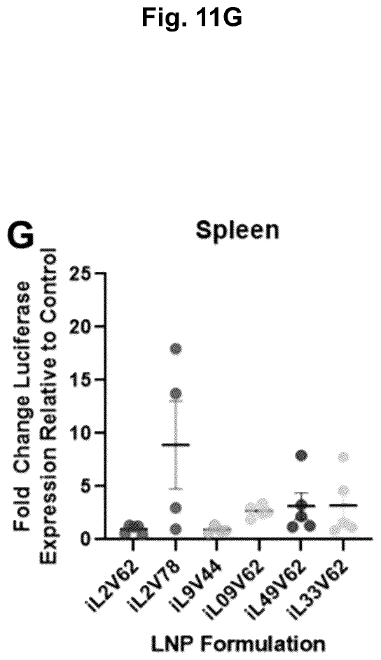

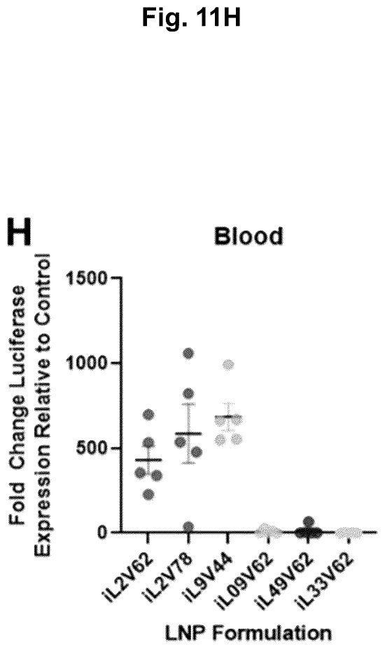

Biodistribution studies show high mRNA expression in the tumor after intratumoral delivery of fLuc LNP (FIG. 11). There was baseline to minimal detection of fLuc expression in any other tissues with all the LNPs examined.

### Example 8

### Intratumoral Delivery of LNP/fLuc to Jax PDX SubQ Developed in NCG Mice Background

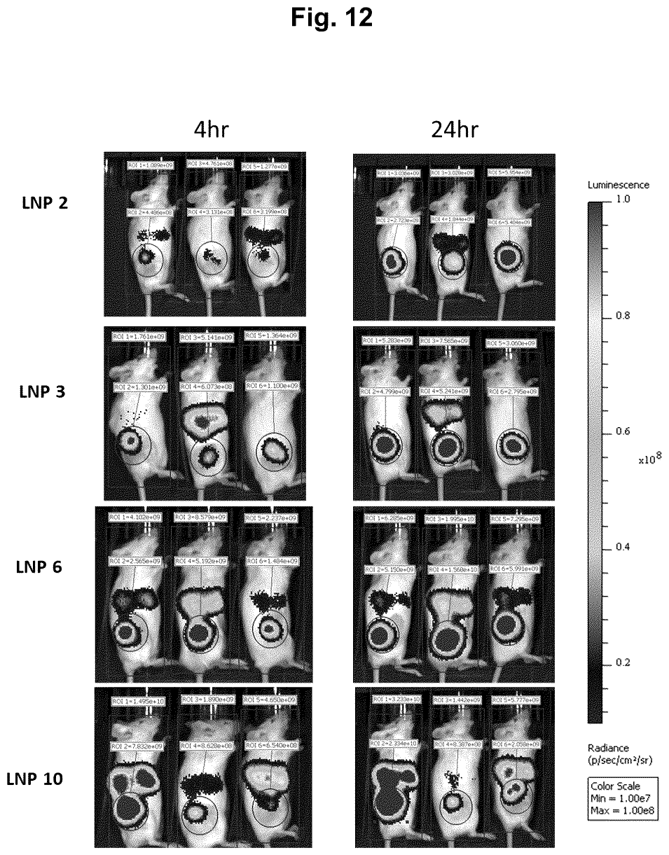

Lipid nanoparticles (LNPs) packaged with firefly luciferase mRNA were used to assess intratumoral delivery and expression within a patient-derived xenograft mouse model. Human lung squamous cell carcinoma tumor fragments were implanted subcutaneously. Once tumors reached 60-150 mm³ in size, mice were injected intratumorally with 2 μg of LNP mixture tumor. Bioluminescence imaging was conducted on mice 4 and 24 hours post injection. FIG. 12 displays time course of the bioluminescent signal post intratumoral injection. Signal from each tumor is quantified by establishing a region of interest (ROI) of the tumor. The ROI value listed as total flux photons of each tumor. Four hours post intratumoral injection in mice across all 4 LNPs tested created a strong signal with comparable ROIs. The signal increased at 24 hours post injection.

### Materials and Methods

### LNP Formulation

The same batch of lipid nanoparticles containing firefly luciferase mRNA from Example 6 were used to repeat these studies. Lipid nanoparticles were purchased from Precision Nanosystems from a proprietary library of ionizable lipids and compositions. Firefly luciferase (FLuc) mRNA (CleanCap FLuc mRNA (5moU)) was purchased from TriLink Biotechnologies (San Diego, CA). FLuc-LNPs were prepared by microfluidic mixing of lipid components and aqueous RNA solution. All LNP formulations were filtered and characterized by Precision Nanosystems.

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of PDX fragments derived from human lung squamous cell carcinoma tumor (obtained from Jackson Laboratories, model TM00244). Tumor growth was measured and estimated using a caliper and calculated as volume (mm³)=(length [mm]×(width [mm]) 2×0.5. When tumors reached ˜60-150 mm³, mice were separated to experimental groups. Intratumoral injections consisted of 2 μg of LNP in a total volume of 25 μL (diluted in PBS as needed). Mice were euthanized and tumors were collected at the indicated time points per experiment.

### Bioluminescence Imaging

Bioluminescence imaging was performed with an IVIS Spectrum imaging system (Caliper Life Sciences). Mice were administered intraperitoneally with d-luciferin (Promega) at a dose of 150 mg/kg. Five minutes after receiving d-luciferin, mice were anesthetized in a chamber with 3% isoflurane and placed on the imaging platform while being maintained on 3% isoflurane via a nose cone. Mice were imaged at 15 minutes post administration of d-luciferin using an exposure time of 1 second or longer. Bioluminescence values were quantified by measuring photon flux (photons/second) in the region of interest where bioluminescence signal emanated using the Living IMAGE Software provided by Caliper (Hopkinton, MA).

### Example 9

Quantitative Analysis of fLuc Expression after Intratumoral Delivery of LNP/fLuc to Jax PDX SubQ Developed in NCG Mice Background

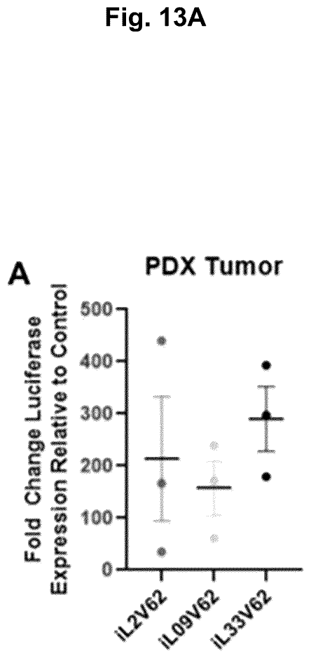

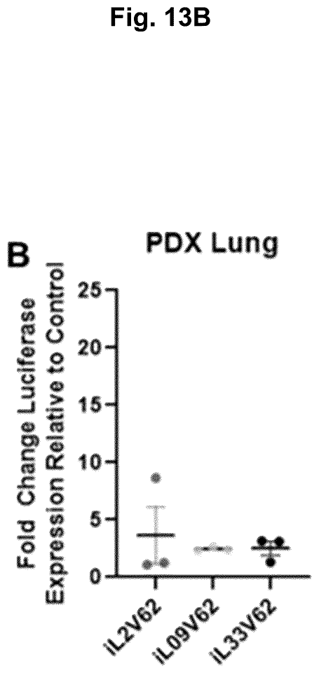

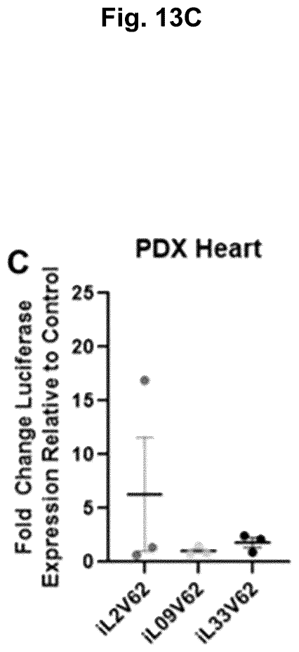

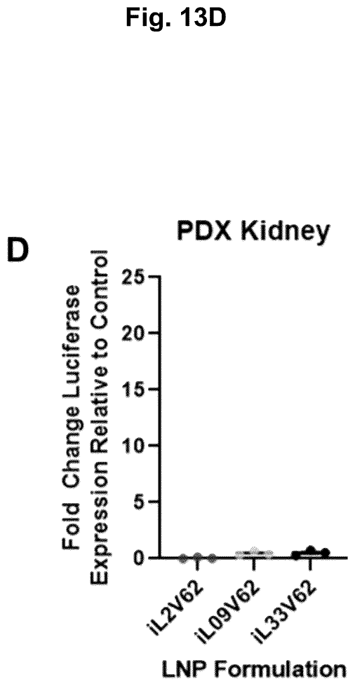

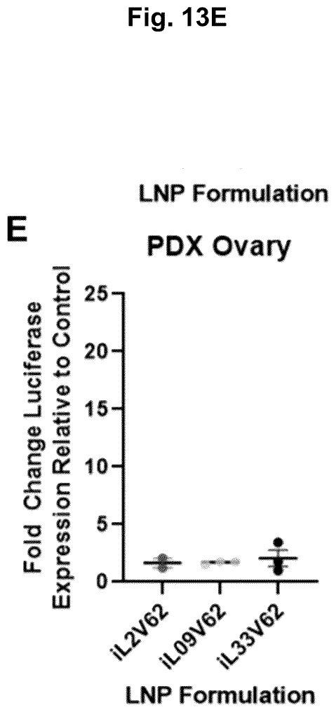

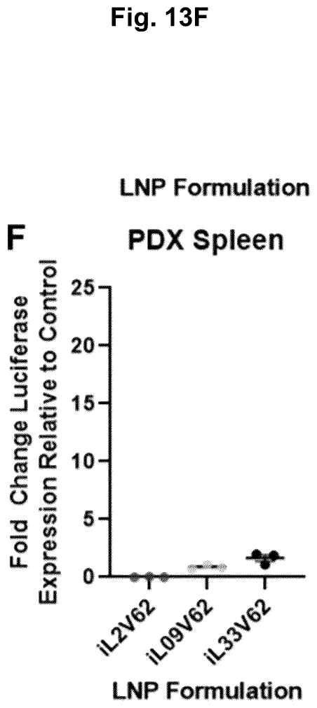

LNP delivery of fLuc mRNA into patient-derived xenograft tumors (Jax PDMR244 obtained from The Jackson Laboratory) was assessed using qRT-PCR. Intratumoral injection of Jax PDMR244 tumors with three different fLuc LNPs also show high expression while only minimal expression in other tissues (FIG. 13). It is consistent with the results from the cell models as shown in FIG. 10 that LNPs delivers the cargo RNAs into the PDX cells for expression.

### Example 10

### Intratumoral Delivery of LNP/fLuc to NCI PDX SubQ Developed in NCG Mice Background

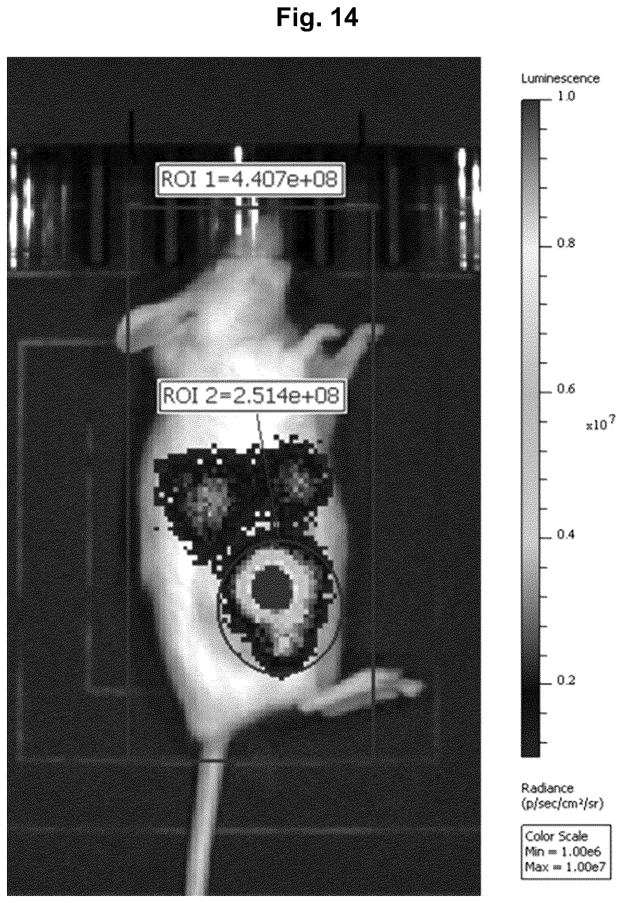

Lipid nanoparticles (LNP) packaged with firefly luciferase mRNA were also used to assess intratumoral delivery and expression within a second patient-derived xenograft mouse model established using PDX fragments obtained from NCI PDMR. Human lung squamous cell carcinoma tumor fragments were implanted subcutaneously. Once tumors reached 60-150 mm³ in size, mice were injected intratumorally with 2 μg of LNP mixture #3. Bioluminescence imaging was conducted on mice 4 and 24 hours post injection. FIG. 14 displays time course of the bioluminescent signal post intratumoral injection. Signal from the tumor is quantified by establishing a region of interest (ROI) of the tumor. The ROI value is listed as total flux photons of each tumor. Twenty-four hours post intratumoral injection in the mouse, there was a strong bioluminescent signal comparable to previous ROIs tested in Examples 6 and 7.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of PDX fragments derived from human lung squamous cell carcinoma tumor (obtained from NCI PDMR, specimen ID 073-R). Tumor growth was measured and estimated using a caliper and calculated as volume (mm³)=(length [mm]×(width [mm]) 2×0.5. When tumors reached ˜60-150 mm³, mice were separated to experimental groups. Intratumoral injections consisted of 2 μg of FLuc-LNP in a total volume of 25 μL (diluted in PBS as needed). Mice were euthanized and tumors were collected at the indicated time points per experiment.

### Bioluminescence Imaging

Bioluminescence imaging was performed with an IVIS Spectrum imaging system (Caliper Life Sciences). Mice were administered intraperitoneally with d-luciferin (Promega) at a dose of 150 mg/kg. Five minutes after receiving d-luciferin, mice were anesthetized in a chamber with 3% isoflurane and placed on the imaging platform while being maintained on 3% isoflurane via a nose cone. Mice were imaged at 15 minutes post administration of d-luciferin using an exposure time of 1 second or longer. Bioluminescence values were quantified by measuring photon flux (photons/second) in the region of interest where bioluminescence signal emanated using the Living IMAGE Software provided by Caliper (Hopkinton, MA).

### Example 11

### Intratumoral Delivery of LNP/CRISPR-Cas9 to H1703 (44-25) SubQ Developed in NCG Mice

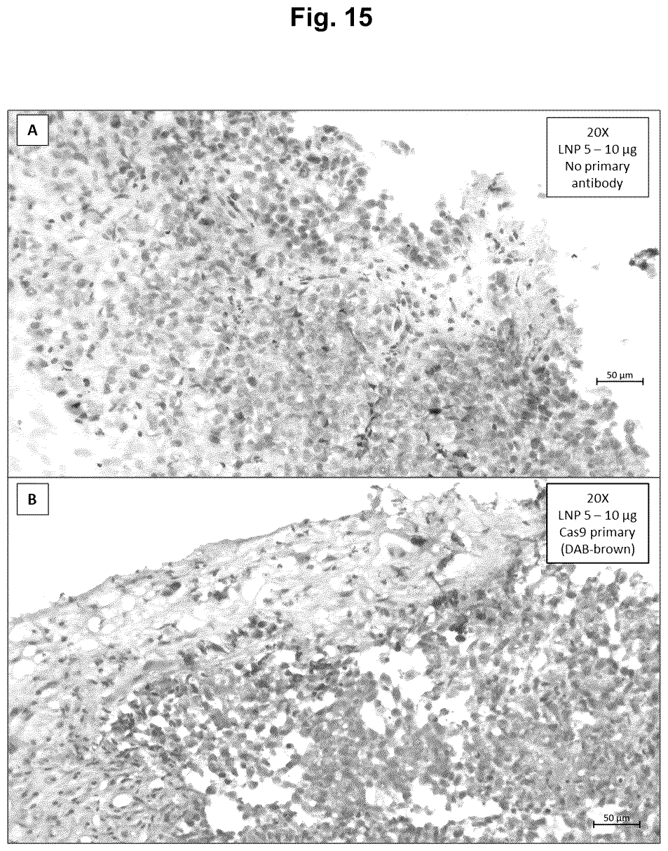

To test efficacy of CRISPR/Cas9 delivery by lipid nanoparticles, Cas9 mRNA and sgRNAs were packaged in lipid nanoparticles, formulated, and provided by Precision Nanosystems. CRISPR-engineered human lung squamous cell carcinoma cell line, H1703 44-25, was implanted subcutaneously. Once tumors reached 100-400 mm³ in size, mice were injected intratumorally with 10 μg and 5 μg of each respective LNP mixture from Table 10. Mice were euthanized and tumors were collected 72 hours or 7 days after intratumoral injection. Tumors were fixed, sliced, and stained for Cas9 protein presence and localization within the tumor. FIG. 15 presents a representative image of control (A) and positively stained tumor sections (B) collected 72 hrs after intratumoral injection of 10 μg of LNP 5. The brown DAB stain (B) represents cells with positive protein expression of Cas9 indicating the transfectability and translation of the lipid nanoparticles and mRNA.

### Materials and Methods

### LNP Formulation

Lipid nanoparticles were formulated at Precision Nanosystems using a proprietary library of ionizable lipids and compositions. Firefly luciferase (FLuc) mRNA (CleanCap FLuc mRNA (5moU)) was purchased from TriLink Biotechnologies (San Diego, CA). FLuc-LNPs were prepared by microfluidic mixing of lipid components and aqueous RNA solution. All LNP formulations were filtered and characterized by Precision Nanosystems.

For this formulation, two different RNA mixes were encapsulated in three separate PNI lipid compositions using the NanoAssemblr Ignite system using the formulation scheme described in Table 9. Formulations were downstream processed and analyzed for size, PDI, zeta potential, total RNA concentration and encapsulation efficiency (% EE) after one freeze-thaw cycle to confirm cryopreservation. Formulation and process parameters were the same as in the screening formulation. Final formulation samples will be shipped at −80° C. (Table 10).

TABLE 9
Parameters used for formulation
of Cas9 mRNA and sgRNA in LNPs

| Formulation | Cas9 mRNA & sgRNA LNPs |
|---|---|
| Instrument | NanoAssemblr ™ Ignite ® |
| Cartridge | NxGen |
| Aqueous stream | RNA mix in PNI formulation buffer |
| Organic stream | PNI's lipid mixes in ethanol |
| Flow rate ratio (FRR) | 3:1 |
| (mRNA solution to lipid solution) | |
| Total flow rate (TFR), mL/min | 12 |
| Start waste, mL | 0.95 |
| End waste, mL | 0.05 |

TABLE 10
Physiochemical Properties of mRNA LNP Formulations

| | | | | Zeta | | Final mRNA |
|---|---|---|---|---|---|---|
| | Storage | Size | | Potential | Encapsulation | concentration |
| Sample ID | Condition | (d · nm) | PDI | (mV) | efficiency (%) | (mg/mL) |
| IL2V62-RNA1-P02E01 | −80° C. | 57 | 0.05 | −4 | 100 | 0.61 |
| IL2V62-RNA2-P02E01 | −80° C. | 56 | 0.07 | −3 | 100 | 0.62 |
| IL2V78-RNA1-P02E01 | −80° C. | 75 | 0.13 | −2 | 99 | 0.57 |
| IL2V78-RNA2-P02E01 | −80° C. | 77 | 0.13 | −2 | 99 | 0.57 |
| IL9V44-RNA1-P02E01 | −80° C. | 81 | 0.07 | −4 | 98 | 0.57 |
| IL9V44-RNA2-P02E01 | −80° C. | 81 | 0.08 | −4 | 98 | 0.60 |

### Immunohistochemistry

Tumor tissues were fixed with 4% paraformaldehyde in 1×PBS overnight at 4° C. Cas9 labelling was performed using immunohistochemistry on fixed tumor tissues. Tumors were frozen and sliced at 10 μm and mounted directly to slides. After blocking with 5% Normal Goat Serum (Vector), rabbit anti-Cas9 primary antibody (Abcam) was applied directly to each section in incubation buffer (1% BSA in PBS) at a 1:100 dilution ratio. The primary antibody was incubated overnight at 4° C. in humidity chambers to prevent drying. Goat anti-rabbit biotinylated secondary (Vector) was applied directly to each section in incubation buffer at a 1:200 dilution ratio. The secondary antibody was incubated at room temperature for 1 hour. ABC Elite HRP kit (Vector) was used for detection with methods following the kit's protocol. DAB was used to develop the stain and slides were counterstained with Hemotoxylin. Slides were then dehydrated in ethanol/xylene and permanently mounted with Acrytol.

### Example 12

qRT-PCR Confirming Cas9 mRNA Expression Present in Tumor

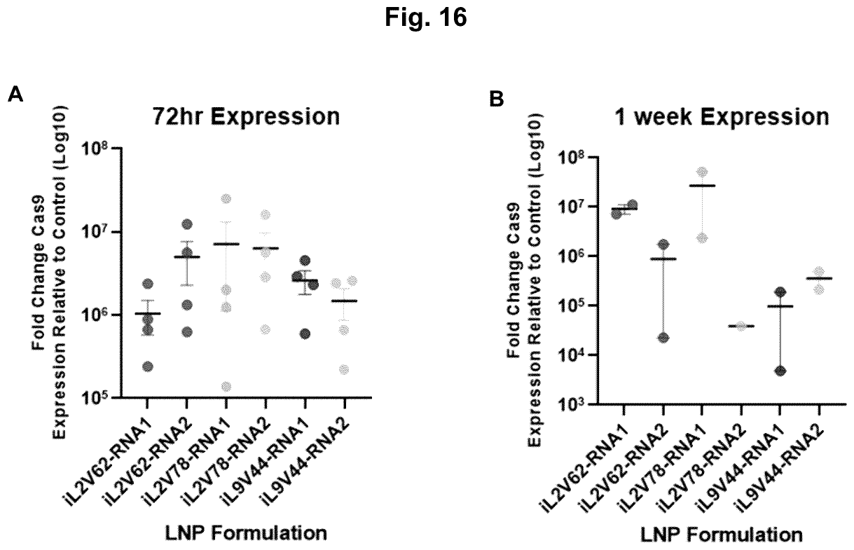

Example 11 demonstrated the presence of Cas9 proteins in the in vivo tumor section. Quantitative analysis was then performed to evaluate the level of Cas9 gene expression with genomic DNAs prepared from the tumor samples post 72 and 168 hrs intratumoral injection of Cas9 LNPs. Examination of Cas9 gene expression showed 10⁶ to 10⁷-fold increase relative to the control tumors with PBS injection. relatively high expression in the tumor (FIG. 16A). Decreased amount of the expression was detected at 168 hrs (FIG. 16B).

### Example 13

### Sanger Sequencing Confirming Editing Activity of CRISPR/Cas9 in Xenograft-Derived Tumors

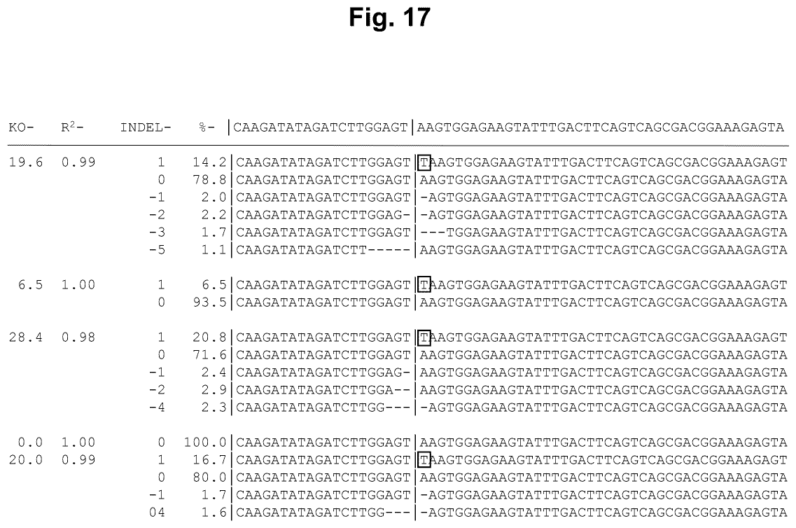

To assess the efficacy of lipid nanoparticle-mediated CRISPR/Cas9 delivery into tumors, tumors from Example 11 were homogenized and genomic DNA was extracted. Once extracted, genomic DNA was amplified with NRF2-locus specific primers (Table 11). Amplicons were then Sanger sequenced and analyzed using DECODR software (Decodr.org: Analyze gene editing efficiency with Sanger sequencing traces) to assess gene editing activity at the target site. FIG. 17 shows a representative DECODR analysis output of select tumor samples sequenced. The results show overall knockout (KO) efficiencies of 6.5%-20.0%. The results confirm the presence and activity of CRISPR/Cas9 within the tumor and at the target site.

### Materials and Methods

### Sanger Sequencing

Genomic DNA is extracted from xenograft tumors using the Qiagen's DNeasy Blood & Tissue kit (Cat. 69504). The region surrounding the CRISPR target site was PCR amplified using Phusion High-Fidelity PCR Master Mix with HF Buffer (Thermofisher cat. F531). The PCR reaction was purified using the QIAquick PCR Purification Kit (Qiagen, Cat. 28106) and Big Dye Terminator PCR was performed using Big DyeTerminator v3.1 (Thermofisher). PCR products were purified once more using the Big Dye Xterminator kit (Thermofisher) and then sequenced using the SeqStudio Genetic Analyzer (Applied Biosystems). Gene editing analysis was conducted using the software program, DECODR, available at https://decodr.org/analyze.

TABLE 11
Sequences of primers.

| SEQ ID | SEQUENCE (5′-3′) | Internal Name |
|---|---|---|
| 24 | CACCATCAACAGTGGCATAATGTGAA | Neh2 PCR FWD |
| 25 | AACTCAGGTTAGGTACTGAACTCATCA | Neh2 PCR REV |

### Example 14

Time Course Study of LNP-Delivered mRNA in Xenograft Mice Model

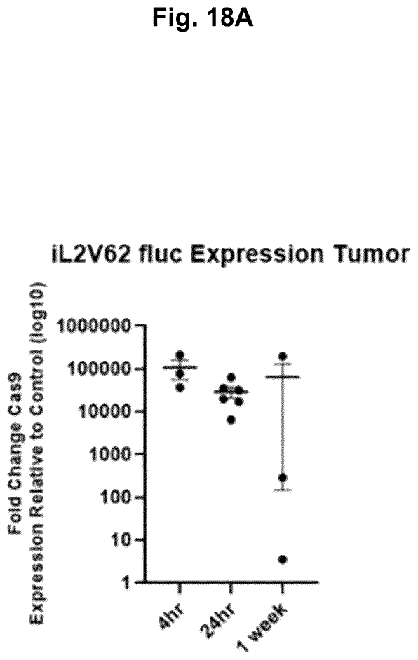

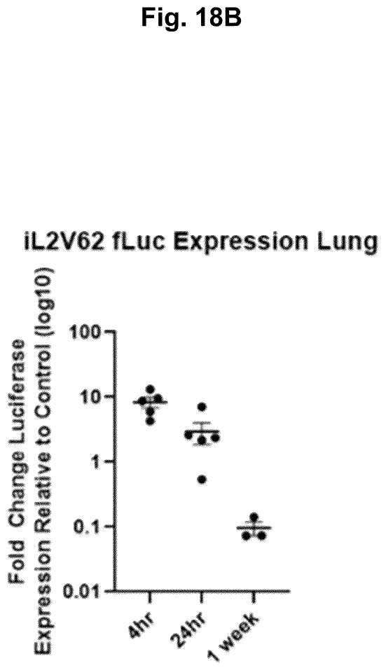

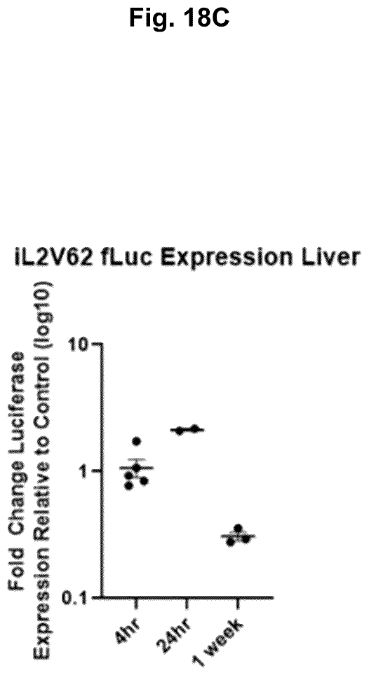

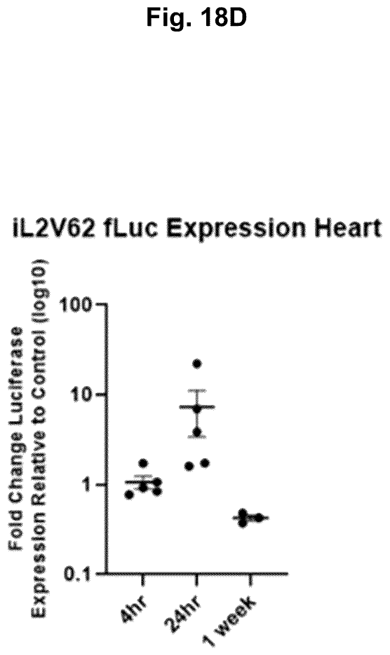

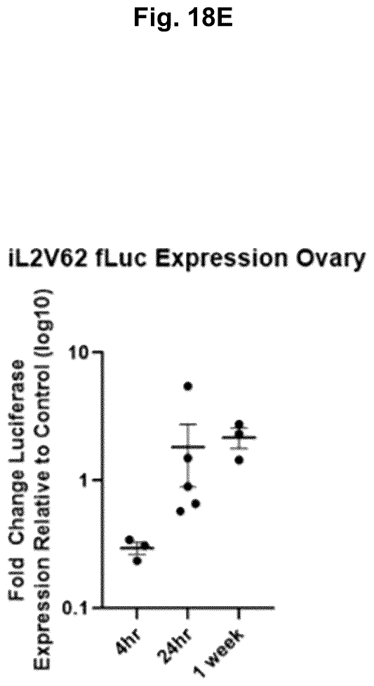

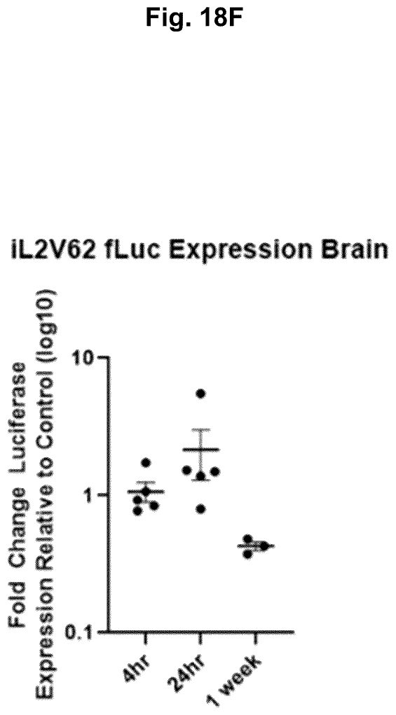

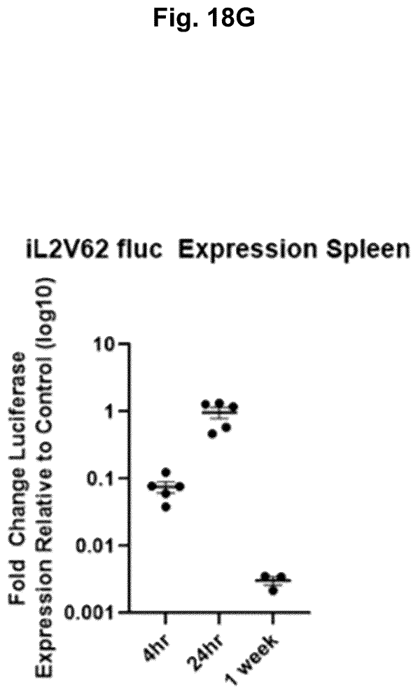

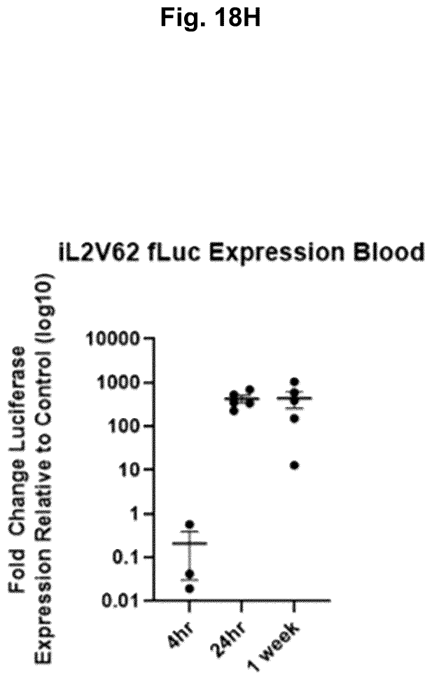

A time course study was carried out to assess the half-life of LNP expression. Mice were intratumorally injected with IL2V62 LNP encapsulated with fLuc mRNA when tumors reached 100-300 mm³, and tissues were collected at 4 hr, 24 hr, and 1 week post injection. The data show that iL2V62 expression was detectable at 4 hr post inject and peaks in expression in tissues around 24 hrs. The expression in the tumor and most of the tissues appears to be prolonged at 1 week although the latter has much less expression (FIG. 18).

### Example 15

In Vitro Evaluation of LNPs Formulated with Cas9 mRNA and sgRNAs

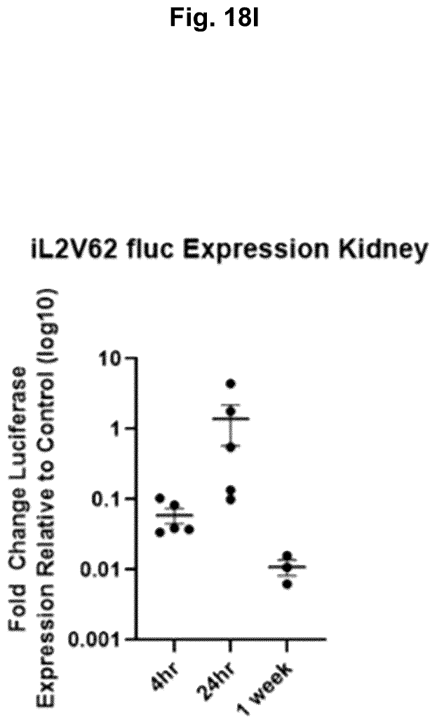

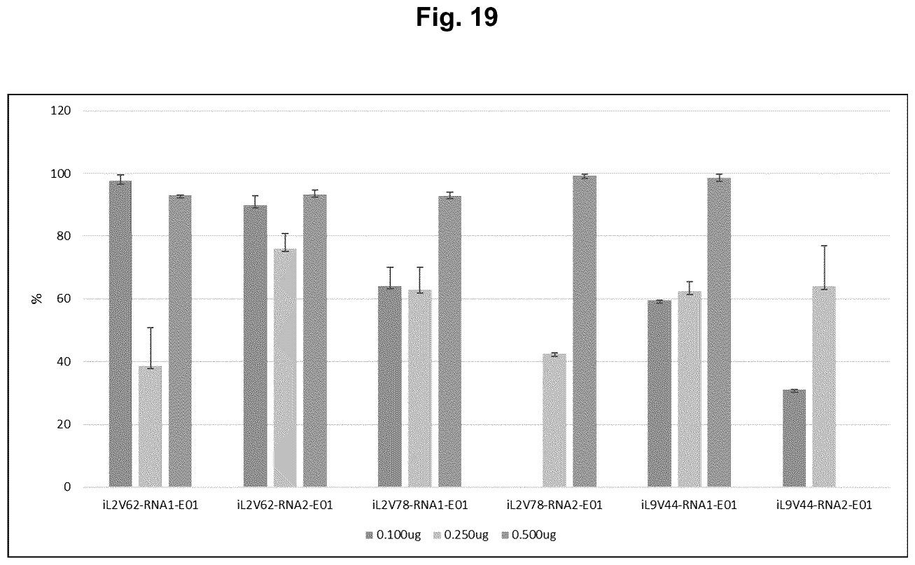

To test efficacy of CRISPR/Cas9 delivery by lipid nanoparticles, Cas9 mRNA and sgRNAs were packaged in lipid nanoparticles, formulated, and provided by Precision Nanosystems (Table 9 & 10). CRISPR-engineered human lung squamous cell carcinoma cell line, H1703 44-25 was plated 50,000 cells per well in 24-well culture plates. Various concentration of LNPs were added to the culture media after 24 hr. Genomic DNAs were prepared from each treatment, sequenced, and analyzed for indel activity at the target site of Cas9 and sgRNA utilizing DECODR. The results showed that up to 96% indel was observed with three different formulations (FIG. 19). It demonstrated that LNPs employed here were capable of delivering Cas9 mRNA and sgRNA into the cells and led to effective CRISPR activity intended on the target site.

1. A method of treating a cancer comprising administering to a subject in need thereof a lipid nanoparticle comprising a therapeutically effective amount of a CRISPR/Cas system comprising (a) one or more nucleic acid sequences encoding one or more guide RNAs (gRNAs) that are complementary to one or more target sequences in a cancer gene of a cancer cell and (b) a nucleic acid sequence encoding a CRISPR-associated endonuclease.

2. The method of claim 1, wherein the one or more gRNAs comprise a trans-activated small RNA (tracrRNA) and a CRISPR RNA (crRNA).

3. The method of claim 1, wherein the one or more gRNAs are one or more single guide RNAs.

4. The method of claim 1, wherein the CRISPR-associated endonuclease is a class 2 CRISPR-associated endonuclease.

5. The method of claim 4, wherein the class 2 CRISPR-associated endonuclease is Cas9 or Cas12a.

6. The method of claim 1, wherein the CRISPR/Cas system is comprised in a ribonucleoprotein (RNP) complex.

7. The method of claim 1, wherein the cancer is resistant to one or more chemotherapeutic agents.

8. The method of claim 1, wherein the cancer is selected from the group consisting of lung cancer, melanoma, esophageal squamous cancer (ESC), head and neck squamous cell carcinoma (HNSCC), and breast cancer.

9. The method of claim 8, wherein the lung cancer is NSCLC.

10. The method of claim 1, further comprising administering one or more chemotherapeutic agents to the subject.

11. The method of claim 10, wherein the one or more chemotherapeutic agents are selected from the group consisting of cisplatin, vinorelbine, carboplatin, and a combination thereof.

12. The method of claim 1, wherein the cancer gene is NRF2 or EGFR.

13. A method of reducing expression of a cancer gene in a cancer cell comprising introducing into the cancer cell a lipid nanoparticle comprising (a) one or more nucleic acid sequences encoding one or more guide RNAs (gRNAs) that are complementary to one or more target sequences in the cancer gene and (b) a nucleic acid sequence encoding a CRISPR-associated endonuclease, whereby the one or more gRNAs hybridize to the cancer gene and the CRISPR-associated endonuclease cleaves the cancer gene.

14. The method of claim 13, wherein the one or more gRNAs comprise a trans-activated small RNA (tracrRNA) and a CRISPR RNA (crRNA).

15. The method of claim 13, wherein the one or more gRNAs are one or more single guide RNAs.

16. The method of claim 13, wherein the CRISPR-associated endonuclease is a class 2 CRISPR-associated endonuclease.

17. The method of claim 16, wherein the class 2 CRISPR-associated endonuclease is Cas9 or Cas12a.

18. The method of claim 13, wherein activity of the cancer gene is reduced in the cancer cell.

19. The method of claim 13, wherein expression or activity of the cancer gene is not completely eliminated in the cancer cell.

20. The method of claim 13, wherein expression or activity of the cancer gene is completely eliminated in the cancer cell.

21. The method of claim 13, wherein the one or more nucleic acid sequences of (a) and the nucleic acid sequence of (b) is comprised in an RNP complex.

22. The method of claim 13, wherein the cancer is resistant to one or more chemotherapeutic agents.

23. The method of claim 13, wherein the cancer is selected from the group consisting of lung cancer, melanoma, esophageal squamous cancer (ESC), head and neck squamous cell carcinoma (HNSCC), and breast cancer.

24. The method of claim 23, wherein the lung cancer is NSCLC.

25. The method of claim 13, further comprising administering one or more chemotherapeutic agents to the subject.

26. The method of claim 25, wherein the one or more chemotherapeutic agents are selected from the group consisting of cisplatin, vinorelbine, carboplatin, and a combination thereof.

27. The method of claim 13, wherein the cancer gene is NRF2 or EGFR.


SEQUENCE LISTING (25 sequences)

SEQ ID NO: 1 - DNA, 24 bp, synthetic construct
  source 1..24: mol_type = other DNA
tttgattgacatactttggaggcg

SEQ ID NO: 2 - DNA, 24 bp, synthetic construct
  source 1..24: mol_type = other DNA
cttactccaagatctatatcttgg

SEQ ID NO: 3 - DNA, 24 bp, synthetic construct
  source 1..24: mol_type = other DNA
atactttggaggcaagatatagag

SEQ ID NO: 4 - DNA, 24 bp, synthetic construct
  source 1..24: mol_type = other DNA
aggcaagatatagatcttggagtg

SEQ ID NO: 5 - DNA, 24 bp, synthetic construct
  source 1..24: mol_type = other DNA
gatatagatcttggagtaagtcgg

SEQ ID NO: 6 - DNA, 24 bp, synthetic construct
  source 1..24: mol_type = other DNA
tgactgaagtcaaatacttctcgg

SEQ ID NO: 7 - DNA, 24 bp, synthetic construct
  source 1..24: mol_type = other DNA
ttcatctagttgtaactgagcgag

SEQ ID NO: 8 - DNA, 24 bp, synthetic construct
  source 1..24: mol_type = other DNA
attcacctgtctcttcatctagtg

SEQ ID NO: 9 - DNA, 24 bp, synthetic construct
  source 1..24: mol_type = other DNA
ctcagttacaactagatgaagagg

SEQ ID NO: 10 - DNA, 24 bp, synthetic construct
  source 1..24: mol_type = other DNA
tgaattgggagaaattcacctgtg

SEQ ID NO: 11 - DNA, 24 bp, synthetic construct
  source 1..24: mol_type = other DNA
caactagatgaagagacaggtgag

SEQ ID NO: 12 - DNA, 24 bp, synthetic construct
  source 1..24: mol_type = other DNA
ataatagctcctcccaaacttgcg

SEQ ID NO: 13 - DNA, 24 bp, synthetic construct
  source 1..24: mol_type = other DNA
tttttcgctcagttacaactagag

SEQ ID NO: 14 - DNA, 24 bp, synthetic construct
  source 1..24: mol_type = other DNA
caactagatgaagagacaggtgag

SEQ ID NO: 15 - DNA, 2446 bp, Homo sapiens
  source 1..2446: mol_type = other DNA
gattaccgagtgccggggagcccggaggagccgccgacgcagccgccaccgccgccgccgccgccaccagagccgccctgtccgcgccgcgcctcggcagccggaacagggccgccgtcggggagccccaacacacggtccacagctcatcatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaa

SEQ ID NO: 16 - DNA, 9905 bp, Homo sapiens
  source 1..9905: mol_type = other DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 17 - protein, 1210 aa, Homo sapiens [sequence omitted]

SEQ ID NO: 18 - DNA, 20 bp, synthetic construct
  source 1..20: mol_type = other DNA
catggccttccgtgttccta

SEQ ID NO: 19 - DNA, 20 bp, synthetic construct
  source 1..20: mol_type = other DNA
acttggcaggtttctccagg

SEQ ID NO: 20 - DNA, 23 bp, synthetic construct
  source 1..23: mol_type = other DNA
tctcctctgacttcaacagcgac

SEQ ID NO: 21 - DNA, 22 bp, synthetic construct
  source 1..22: mol_type = other DNA
ccctgttgctgtagccaaattc

SEQ ID NO: 22 - DNA, 20 bp, synthetic construct
  source 1..20: mol_type = other DNA
tgatgtaccggttcgaggag

SEQ ID NO: 23 - DNA, 20 bp, synthetic construct
  source 1..20: mol_type = other DNA
ttgtcgatcagggtgctctt

SEQ ID NO: 24 - DNA, 26 bp, synthetic construct
  source 1..26: mol_type = other DNA
caccatcaacagtggcataatgtgaa

SEQ ID NO: 25 - DNA, 27 bp, synthetic construct
  source 1..27: mol_type = other DNA
aactcaggttaggtactgaactcatca